(19) United States
(12) Patent Application Publication
(10) Pub. No.: US 2022/0403022 A1
(43) Pub. Date: Dec. 22, 2022

(54) Anti-TIGIT Antibodies and Uses Thereof

(71) Applicant: Merck Patent GmbH, Darmstadt (DE)

(72) Inventors: Dong ZHANG, Westwood, MA (US); Christie KELTON, Hopkinton, MA (US); Liwei LI, Arlington, MA (US); David NANNEMANN, Rio Rancho, NM (US); Qi AN, Nashua, NH (US); Christel IFFLAND, Arlington, MA (US); Xinyan ZHAO, Bedford, MA (US); Johannes YEH, Billerica, MA (US)

(21) Appl. No.: 17/774,685

(22) PCT Filed: Nov. 5, 2020
(86) PCT No.: PCT/US20/59139

§ 371 Date: May 5, 2022

Related U.S. Application Data
(60) Provisional application No. 63/048,351, filed on Jul. 6, 2020.
(60) Provisional application No. 62/930,651, filed on Nov. 5, 2019.

(51) Int. Cl. C07K 16/28; A61K 39/395; A61P 35/00; C12N 5/00

(52) U.S. Cl. CPC C07K 16/28; A61K 39/3955; A61P 35/00; C12N 5/00; C07K 2317/21; A61K 39/00

(57) ABSTRACT

The present application relates to anti-TIGIT antibodies or antigen binding fragments thereof, nucleic acid encoding the same, therapeutic compositions thereof, and their use to enhance T-cell function to upregulate cell-mediated immune responses and for the treatment of T cell dysfunctional disorders, such as tumor immunity, for the treatment of infectious diseases and cancer.

### SEQUENCE LISTING

The instant application contains a Sequence Listing which has been submitted electronically in ASCII format and is hereby incorporated by reference in its entirety. Said ASCII copy, created on Nov. 5, 2020, is named P19-193_WO_SL.txt and is 40,940 bytes in size.

### FIELD OF THE INVENTION

The present application relates to anti-TIGIT antibodies or antigen binding fragments thereof, nucleic acid encoding the same, therapeutic compositions thereof, and their use to enhance T-cell function to upregulate cell-mediated immune responses and for the treatment of T cell dysfunctional disorders, such as tumor immunity, for the treatment of infectious diseases and cancer.

### BACKGROUND OF THE INVENTION

Lymphocyte Development and Activation

The two major types of lymphocytes in humans are T (thymus-derived) and B (bone marrow derived. These cells are derived from hematopoietic stem cells in the bone marrow and fetal liver that have committed to the lymphoid development pathway. The progeny of these stem cells follow divergent pathways to mature into either B or T lymphocytes. Human B-lymphocyte development takes place entirely within the bone marrow. T cells, on the other hand, develop from immature precursors that leave the marrow and travel through the bloodstream to the thymus, where they proliferate and differentiate into mature T lymphocytes.

Mature lymphocytes that emerge from the thymus or bone marrow are in a quiescent, or “resting” state, i.e., they are mitotically inactive. When dispersed into the bloodstream, these “naive” or “virgin” lymphocytes, travel into various secondary or peripheral lymphoid organs, such as the spleen, lymph nodes or tonsils. Most virgin lymphocytes have an inherently short life span and die without a few days after leaving the marrow or thymus. However, if such a cell receives signals that indicate the presence of an antigen, they may activate and undergo successive rounds of cell division. Some of the resulting progeny cells then revert to the resting state to become memory lymphocytes—B and T cells that are essentially primed for the next encounter with the stimulating allergen. The other progeny of activated virgin lymphocytes are effector cells, which survive for only a few days, but carry out specific defensive activities.

Lymphocyte activation refers to an ordered series of events through which a resting lymphocyte passes as it is stimulated to divide and produce progeny, some of which become effector cells. A full response includes both the induction of cell proliferation (mitogenesis) and the expression of immunologic functions. Lymphocytes become activated when specific ligands bind to receptors on their surfaces. The ligands are different for T cells and B cells, but the resulting intracellular physiological mechanisms are similar.

Some foreign antigens themselves can induce lymphocyte activation, especially large polymeric antigens that cross-link surface immunoglobulins on B-cells, or other glycoproteins on T-cells. However, most antigens are not polymeric and even direct binding to B-cells in large numbers fail to result in activation. These more common antigens activate B cells when they are co-stimulated with nearby activated helper T-lymphocytes. Such stimulation may occur from lymphokines secreted by the T-cell, but is transmitted most efficiently by direct contact of the B cell with T-cell surface proteins that interact with certain B-cell surface receptors to generate a secondary signal.

T-Cells

T lymphocytes do not express immunoglobulins, but instead detect the presence of foreign substances by way of surface proteins called T-cell receptors (TCR). These receptors recognize antigens by either direct contact or through influencing the activity of other immune cells. Together with macrophages, T cells are the primary cell type involved in the cell-mediated immunity.

Unlike B-cells, T-cells can detect foreign substances only in specific contexts. In particular, T-lymphocytes will recognize a foreign protein only if it first cleaved into small peptides, which are then displayed on the surface of a second host cell, called an antigen-presenting cell (APC). Many types of host cells can present antigens under some conditions, but certain types are more specifically adapted for this purpose and are particularly important in controlling T-cell activity, including macrophages and other B-cells. Antigen presentation depends in part on specific proteins, called major histocompatibility complex (MHC) proteins, on the surface of the presenting cells. Thus, to stimulate cell-mediated immunity, foreign peptides must be presented to T-cells in combination with MHC peptides, and this combination must be recognized by a T-cell receptor.

There are two significant T-cell subsets: cytotoxic T lymphocytes (T_{c} cells or CTLs) and helper T cells (T_{H}) cells, which can roughly be identified on the basis of cell surface expression of the marker CD8 and CD4. T_{c} cells are important in viral defense and can kill viruses directly by recognizing certain cell surface expressed viral peptides. T_{H} cells promote proliferation, maturation and immunologic function of other cell types, e.g. lymphokine secretion to control activities of B cells, macrophages and cytotoxic T cells.

Both virgin and memory T-lymphocytes ordinarily remain in the resting state, and in this state they do not exhibit significant helper or cytotoxic activity. When activated, these cells undergo several rounds of mitotic division to produce daughter cells. Some of these daughter cells return to the resting state as memory cells, but others become effector cells that actively express helper or cytotoxic activity. These daughter cells resemble their parents: CD4+ cells can only produce CD4+ progeny, while CD8+ cells yield only CD8+ progeny. Effector T-cells express cell surface markers that are not expressed on resting T-cells, such as CD25, CD28, CD29, CD40L, transferrin receptors and class II MHC proteins. When the activating stimuli is withdrawn, cytotoxic or helper activity gradually subsides over a period of several days as the effector cells either die or revert to the resting state.

Similar to B-cell activation, T-lymphocyte responses to most antigens also require two types of simultaneous stimuli. The first is the antigen, which if appropriately displayed by MHC proteins on an antigen-presenting cell, can be recognized and bound by T-cell receptors. While this antigen-MHC complex does send a signal to the cell interior, it is usually insufficient to result in T-cell activation. Full activation, such as occurs with helper T-cells, requires co-stimulation with other specific ligands called co-stimulators that are expressed on the surface of the antigen-presenting cell. Activation of a cytotoxic T cell, on the other hand, generally requires IL-2, a cytokine secreted by activated helper T cells.

Immune Modulatory Receptors

A key factor for enabling tumor immunotherapy emerged from discoveries that inhibitory immune modulatory receptors (IMRs), that generally function as immune checkpoints to maintain self-tolerance, are central to the ability of tumor microenvironments to evade immunity. Blockade of inhibitory IMRs appears to unleash potent tumor-specific immune responses more effectively than direct stimulation of tumor-immunity with activating cytokines or tumor vaccines, and this approach has the potential to transform human cancer therapy. An important implication and opportunity now arises for the potential to develop new antibody antagonists for other IMRs and to combine antagonist antibodies to more than one IMR in order to increase the proportion of responders in oncology clinical trials, as well as, expand upon oncology indications in which tumor immunotherapy treatments are effective.

Significantly, inhibitory IMRs and ligands that regulate cellular immunity are commonly overexpressed on tumor cells and tumor associated macrophages (TAMs). Notably, overexpression of PD-L1 in tumors is associated with tumor specific T cell exhaustion and a poor prognosis. Blockade of PD-1/PD-L1 ligation in clinical trials resulted in durable tumor regression responses in a substantial proportion of patients. A recent report demonstrated that co-expression of PD-1 and another inhibitory IMR (TIM-3) in melanoma patient derived tumor-specific CD8+ T cells was associated with more dysfunctional T cell exhaustion phenotypes compared to cells expressing either IMR alone. Moreover, several reports using pre-clinical tumor models demonstrated blockade of multiple IMRs, including PD-1, TIM-3, LAG-3 and CTLA-4 more effectively induced anti-tumor responses than antagonizing PD-1 alone. These results underscore the importance of further investigating IMR pathways.

TIGIT Structure and Signaling

TIGIT (T cell immunoreceptor with Ig and ITIM domains) is an immunomodulatory receptor expressed primarily on activated T cells and NK cells. TIGIT is also known as VSIG9; VSTM3; and WUCAM. Its structure shows one extracellular immunoglobulin domain, a type 1 transmembrane region and two ITIM motifs. TIGIT forms part of a costimulatory network that consists of positive (CD226) and negative (TIGIT) immunomodulatory receptors on T cells, and ligands expressed on APCs (CD155/PVR and CD112).

An important feature in the structure of TIGIT is the presence of an immunoreceptor tyrosine-based inhibition motif (ITIM) in its cytoplasmic tail domain. As with PD-1 and CTLA-4, the ITIM domain in the cytoplasmic region of TIGIT is predicted to recruit tyrosine phosphatases, such as SHP-1 and SHP-2, and subsequent de-phosphorylation of tyrosine residues within the immunoreceptor tyrosine-base activation motifs (IT AM) on T cell receptor (TCR) subunits. Hence, ligation of TIGIT by receptor-ligands CD155 and CD112 expressed by tumor cells or TAMS may contribute to the suppression of TCR-signaling and T cell activation, which is essential for mounting effective anti-tumor immunity. Thus, an antagonist antibody specific for TIGIT could inhibit the CD155 and CD112 induced suppression of T cell responses and enhance anti-tumor immunity. It is an object of the present invention to obtain an anti-TIGIT antibody that can be used for the treatment of infectious diseases and cancer, either alone or in combination with other reagents. The amino acid sequence of human TIGIT is the following (Genbank accession number NP_776160):

(SEQ ID NO: 1)
MRWCLLLIWAQGLRQAPLASGMMTGTIETTGNISAEKGGSIILQCHLSS
TTAQVTQVNWEQQDQLLAICNADLGWHISPSFKDRVAPGPGLGLTLQSL
TVNDTGEYFCIYHTYPDGTYTGRIFLEVLESSVAEHGARFQIPLLGAMA
ATLVVICTAVIVVVALTRKKKALRIHSVEGDLRRKSAGQEEWSPSAPSP
PGSCVQAEAAPAGLCGEQRGEDCAELHDYFNVLSYRSLGNCSFFTETG

TIGIT and CD96 together with CD226 (DNAM-1) form a pathway that closely resembles the CD28/CTLA-4 pathway. Similar to CD28 and CTLA-4, CD226 functions as a costimulatory receptor that shares ligands with TIGIT and CD96, which function as co-inhibitory receptors. CD226 and TIGIT bind to two nectin and nectin-like (necl) proteins: PVR (CD155, necl-5) and CD112 (PVRL2, nectin-2). CD96 shares binding of CD155 with CD226 and TIGIT, but also binds to CD111.

TIGIT is upregulated on CD8+ T cells upon activation (Joller et al., J Immunol 186: 1338-1342, 2011). Others and we have shown that TIGIT expression is highly enriched on CD8+tumor infiltrating lymphocytes (TILs) in mice. (Johnston et al Cancer Cell. 2014; 26:923-937, Kurtulus et al, J Clin Investig. 2015; 125:4053-4062) Importantly, TIGIT is also highly expressed on CD8+TILs in non-small cell lung cancer, colon cancer, and melanoma. (Chauvin et al., J Clin Investig. 2015; 125:2046-2058, Johnston et al Cancer Cell. 2014; 26:923-937) and in the peripheral blood mononuclear cells (PBMCs) of acute myelogenous leukemia (AML) patients (Kong et al. Clin Cancer Res. 2016; 22:3057-3066). Within CD8+TILs, TIGIT marks a subset of CD8+ T cells that co-express the co-inhibitory receptors Tim-3 and PD-1, are poor producers of TNF-α and IL-2 and have decreased cytotoxicity compared to TIGIT-CD8+TILs (Kurtulus et al, J Clin Investig. 2015; 125:4053-4062). Importantly, TIGIT expression is also significantly higher in PD-1+Tim-3+CD8+TILs in melanoma patients, and TIGIT correlates with poor cytokine production in both melanoma and AML patients (Kong et al. Clin Cancer Res. 2016; 22:3057-3066). Together, these data indicate that TIGIT is found on CD8+TILs that exhibit dysfunctional phenotype in both mouse and human.

Several lines of evidence indicate that TIGIT limits the effector functions and expansion of CD8+ T cells. Kurtulus et al. showed that CD8+TILs from Tigit−/− mice exhibit enhanced cytotoxic and proliferative capacity (J Clin Investig. 2015; 125: 4053-4062). Similarly, knockdown of TIGIT in CD8+ T cells from AML patients results in reversal of functional defects (Kong et al. Clin Cancer Res. 2016; 22:3057-3066). Moreover, others have shown that blockade of TIGIT synergizes with PD-1 blockade to increase production of IFN-γ and TNF-α by CD8+ T cells in murine colon cancer TILs (Johnston et al Cancer Cell. 2014; 26:923-937) and enhanced antigen-specific proliferation, cytokine production, and degranulation in CD8+ T cells from the PBMC and TILs of melanoma patients (Chauvin et al., J Clin Investig. 2015; 125:2046-2058). Together, these data support a role for TIGIT in restraining the expansion and effector functions of CD8+ T cells in the tumor context (Johnston et al Cancer Cell. 2014; 26:923-937, 41 and Kong et al. Clin Cancer Res. 2016; 22:3057-3066).

In particular, the inhibition of TIGIT signaling has been proposed as a means to enhance T cell immunity for the treatment of cancer (e.g., tumor immunity) and infection, including both acute and chronic (e.g., persistent) infection. Inhibitors blocking the TIGIT signaling are known from, e.g. WO16028656 and WO16011264. However, as an optimal therapeutic directed to a target in this pathway has yet to be commercialized, a significant unmet medical need exists.

### DESCRIPTION OF THE INVENTION

It is an objective of the present invention to provide for anti-TIGIT antibodies, including nucleic acids encoding and compositions containing such antibodies, and for their use to enhance anti-tumor immunity. Surprisingly it was found that the anti-TIGIT antibodies of this invention are particularly potent in mediating antibody dependent cell-mediated cytotoxicity (ADCC) and potentiating mixed lymphocyte reaction (MLR) as compared to previously described anti-TIGIT antibodies tested. Moreover, the antibodies not only block the interaction between human TIGIT and human PVR (CD155), but also the interactions between the respective cynomolgus monkey proteins. Finally, the antibodies of this invention bind a unique epitope that comprises the residues Q53, T55, Y113, and P114 of human TIGIT.

In one aspect, the invention provides for an isolated heavy chain variable region polypeptide comprising an HVR-H1, HVR-H2 and HVR-H3 sequence, wherein:

(a) the HVR-H1 sequence is GYTFTX₁YP (SEQID NO:36);

(b) the HVR-H2 sequence is INTNTGNP (SEQID NO:14)

(c) the HVR-H3 sequence is ARX₂GX₃X₄X₅X₆X₇X₈X₉X₁₀X₁₁X₁₂X₁₃ (SEQID NO:37);

further wherein: X₁ is S or A; X₂ is V or T; X₃ is G or Y; X₄ is Y, S or F; X₅ is S, G or T; X₆ is V, S or G; X₇ is D, Y or P; X₈ is E, D or Y; X₉ is Y or W; X₁₀ is A, F or S; X₁₁ is F or D; X₁₂ is D or P; X₁₃ is V, I or absent.

In an embodiment, X₁ is S or A; X₂ is V or T; X₃ is G or Y; X₄ is Y or S; X₅ is S or G; X₆ is V or S; X₇ is D or Y; X₈ is E; X₉ is Y; X₁₀ is A or F; X₁₁ is F; X₁₂ is D; X₁₃ is V or I.

In another embodiment, X₁ is S; X₂ is V or T; X₃ is G; X₄ is Y; X₅ is S or G; X₆ is V; X₇ is D or Y; X₈ is E; X₉ is Y; X₁₀ is A; X₁₁ is F; X₁₂ is D; X₁₃ is V or I.

In yet another embodiment, X₁ is S; X₂ is V; X₃ is G; X₄ is Y; X₅ is S; X₆ is V; X₇ is D; X₈ is E; X₉ is Y; X₁₀ is A; X₁₁ is F; X₁₂ is D; X₁₃ is V.

In an aspect, the polypeptide 4 further comprises variable region heavy chain framework sequences HC-FR1, HC-FR2, HC-FR3 and HC-FR4, juxtaposed between the HVRs, thus forming the sequence of the formula: (HC-FR1)-(HVR-H1)-(HC-FR2)-(HVR-H2)-(HC-FR3)-(HVR-H3)-(HC-FR4).

In an embodiment, the framework sequences are derived from human consensus framework sequences or human germline framework sequences.

In another embodiment, at least one of the framework sequences is the following:

(SEQ ID NO: 2)
HC-FR1 is QVQLVQSGSELKKPGASVKVSCKAS;
(SEQ ID NO: 3)
HC-FR2 is MNWWRQAPGQGLEWMGW;
(SEQ ID NO: 4)
HC-FR3 is TYAQGFTGRFVFSLDTSVSTAYLQISSLKAEDTAVYYC;
(SEQ ID NO: 5)
HC-FR4 is WGQGTLVTVSS.

In further embodiments the polypeptide comprises at least a C_{H}1 domain, and optionally, a C_{H}2 and a C_{H}3 domain.

In a further aspect, the heavy chain polypeptide is further combined with a variable region light chain comprising an HVR-L1, HVR-L2 and HVR-L3, wherein:

(a) the HVR-L1 sequence is QGISSY (SEQID NO:6);

(b) the HVR-L2 sequence is AAS (SEQID NO:7);

(c) the HVR-L3 sequence is X₁₄QX₁₅X₁₆X₁₇X₁₈X₁₉X₂₀ (SEQID NO:38); further wherein X₁₄ is Q, G or H; X₁₅ is L, V or T; X₁₆ is N, S, I or M; X₁₇ is S, R, or F; X₁₈ is Y or R; X₁₉ is P or L; X₂₀ is T or A.

In an embodiment X₁₄ is Q or G; X₁₅ is L or V; X₁₆ is N or S; X₁₇ is S or R; X₁₈ is Y; X₁₉ is P; X₂₀ is T.

In another embodiment, X₁₄ is Q; X₁₅ is L; X₁₆ is S; X₁₇ is S; X₁₈ is Y; X₁₉ is P; X₂₀ is T (SEQID NO:8).

In an aspect, the light chain further comprising variable region light chain framework sequences LC-FR1, LC-FR2, LC-FR3 and LC-FR4, juxtaposed between the HVRs, thus forming the sequence of the formula: (LC-FR1)-(HVR-L1)-(LC-FR2)-(HVR-L2)-(LC-FR3)-(HVR-L3)-(LC-FR4).

In an embodiment, the light chain framework sequences are derived from human consensus framework sequences or human germline framework sequences.

In another embodiment, the light chain framework sequences are kappa light chain sequences.

In yet another embodiment, at least one of the light chain framework sequences is the following:

LC-FR1 sequence is DIQLTQSPSFLSASVGDRVTITCRAS (SEQID NO:9);

LC-FR2 sequence is LAWYQQKPGKAPKLLIY (SEQID NO:10);

LC-FR3 sequence is TLQSGVPSRFSGSGSGTEFTLTISSLQPEDFATYYC (SEQID NO:11);

LC-FR4 sequence is FGGGTKVEIK (SEQID NO:12).

In a further embodiment, the light chain polypeptide comprises a CL domain.

In another aspect, the invention provides an isolated anti-TIGIT antibody or antigen binding fragment thereof comprising a heavy chain and a light chain variable region sequence, wherein:

(a) the heavy chain comprises an HVR-H1, HVR-H2 and HVR-H3, wherein further: (i) the HVR-H1 sequence is GYTFTX₁YP; (ii) the HVR-H2 sequence is INTNTGNP (SEQID NO:14); (iii) the HVR-H3 sequence is ARX₂GX₃X₄X₅X₆X₇X₈X₉X₁₀X₁₁X₁₂X₁₃ (SEQID NO:37);

(b) the light chain comprises an HVR-L1, HVR-L2 and HVR-L3, wherein further: (iv) the HVR-L1 sequence is QGISSY (SEQID NO:6); (v) the HVR-L2 sequence is AAS (SEQID NO:7); (vi) the HVR-L3 sequence is X₁₄QX₁₅X₁₆X₁₇X₁₈X₁₉X₂₀ (SEQID NO:38);

wherein further X₁ is S or A; X₂ is V or T; X₃ is G or Y; X₄ is Y, S or F; X₅ is S, G or T; X₆ is V, S or G; X₇ is D, Y or P; X₈ is E, D or Y; X₉ is Y or W; X₁₀ is A, F or S; X₁₁ is F or D; X₁₂ is D or P; X₁₃ is V, I or absent; X₁₄ is Q, G or H; X₁₅ is L, V or T; X₁₆ is N, S, I, or M; X₁₇ is S, R, or F; X₁₈ is Y or R; X₁₉ is P or L; X₂₀ is T or A.

In an embodiment, X₁ is S or A; X₂ is V or T; X₃ is G or Y; X₄ is Y or S; X₅ is S or G; X₆ is V or S; X₇ is D or Y; X₈ is E; X₉ is Y; X₁₀ is A or F; X₁₁ is F; X₁₂ is D; X₁₃ is V or I; X₁₄ is Q or G; X₁ is L or V; X₁ is N or S; X₁₇ is S or R; X₁₈ is Y; X₁₉ is P; X₂₀ is T.

In another embodiment, X₁ is S; X₂ is V or T; X₃ is G; X₄ is Y; X₅ is S or G; X₆ is V; X₇ is D or Y; X₈ is E; X₉ is Y; X₁₀ is A; X₁₁ is F; X₁₂ is D; X₁₃ is V or I; X₁₄ is Q; X₁₅ is L; X₁₆ is S; X₁₇ is S; X₁₈ is Y; X₁₉ is P; X₂₀ is T.

In yet another embodiment, X₁ is S; X₂ is V; X₃ is G; X₄ is Y; X₅ is S; X₆ is V; X₇ is D; X₈ is E; X₉ is Y; X₁ is A; X₁₁ is F; X₁₂ is D; X₁₃ is V; X₁₄ is Q; X₁₅ is L; X₁₆ is S; X₁₇ is S; X₁₈ is Y; X₁₉ is P; X₂₀ is T.

In yet another specific embodiment, the invention provides an isolated anti-TIGIT antibody or antigen binding fragment thereof wherein

(a) the HVR-H1 sequence is GYTFTSYP (SEQID NO:13),

(b) the HVR-H2 sequence is INTNTGNP (SEQID NO:14),

(c) the HVR-H3 sequence is ARVGGYSVDEYAFDV (SEQID NO:15); and wherein

(d) the HVR-L1 sequence is QGISSY (SEQID NO:6),

(e) the HVR-L2 sequence is AAS (SEQID NO:7),

(f) the HVR-L3 sequence is QQLSSYPT (SEQID NO:8).

In a further aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4).

In an embodiment, the framework sequences are derived from human consensus framework sequences or human germline sequences.

In another embodiment, the heavy chain framework sequences are:

HIHC-FR1 is
(SEQ ID NO: 2)
QVQLVQSGSELKKPGASVKVSCKAS;
HC-FR2 is
(SEQ ID NO: 3)
MNWWRQAPGQGLEWMGW;
HC-FR3 is
(SEQ ID NO: 4)
TYAQGFTGRFVFSLDTSVSTAYLQISSLKAEDTAVYYC;
HC-FR4 is
(SEQ ID NO: 5)
WGQGTLVTVSS.

In a yet further embodiment, the light chain framework sequences are kappa light chain sequences.

In a still further embodiment, one or more of the light chain framework sequences are:

LC-FR1 is
(SEQ ID NO: 9)
DIQLTQSPSFLSASVGDRVTITCRAS;
LC-FR2 is
(SEQ ID NO: 10)
LAWYQQKPGKAPKLLIY;
LC-FR3 is
(SEQ ID NO: 11)
TLQSGVPSRFSGSGSGTEFTLTISSLQPEDFATYYC
LC-FR4 is
(SEQ ID NO: 12)
FGGGTKVEIK.

In a still further specific embodiment, the invention provides an isolated anti-TIGIT antibody or antigen binding fragment thereof wherein:

(a) the variable heavy chain framework sequences are the following:

(i) HC-FR1 is
(SEQ ID NO: 2)
QVQLVQSGSELKKPGASVKVSCKAS;
(ii) HC-FR2 is
(SEQ ID NO: 3)
MNWWRQAPGQGLEWMGW;
(iii) HC-FR3 is
(SEQ ID NO: 4)
TYAQGFTGRFVFSLDTSVSTAYLQISSLKAEDTAVYYC;
(iv) HC-FR4 is
(SEQ ID NO: 5)
WGQGTLVTVSS;

and

(b) the variable light chain framework sequences are the following:

(i) LC-FR1 sequence is
(SEQ ID NO: 9)
DIQLTQSPSFLSASVGDRVTITCRAS;
(ii) LC-FR2 sequence is
(SEQ ID NO: 10)
LAWYQQKPGKAPKLLIY;
(iii) LC-FR3 sequence is
(SEQ ID NO: 11)
TLQSGVPSRFSGSGSGTEFTLTISSLQPEDFATYYC;
(iv) LC-FR4 sequence is
(SEQ ID NO: 12)
FGGGTKVEIK.

In a further aspect, the invention provides an isolated anti-TIGIT antibody or antigen binding fragment thereof, having the HC-FR and LC-FR sequences as disclosed above, selected from the following:

i) an antibody, wherein the HVR-H1, HVR-H2, HVR-H3 sequences are selected from one of the ID's shown in Table 2 of Example 1, and wherein

(a) the HVR-L1 sequence is QGISSY (SEQID NO:6),

(b) the HVR-L2 sequence is AAS (SEQID NO:7),

(c) the HVR-L3 sequence is QQLNSYPT (SEQID NO:8);

ii) an antibody wherein the HVR-L1, HVR-L2, HVR-L3 sequences are selected from one of the ID's shown in Table 3 of Example 1, and wherein

(a) the HVR-H1 sequence is GYTFTSYP (SEQID NO:13),

(b) the HVR-H2 sequence is INTNTGNP (SEQID NO:14),

(c) the HVR-H3 sequence is ARVGGYSVDEYAFDV (SEQID NO:15); or

iii) an antibody chosen from Table 4 of Example 1.

In a further aspect, the heavy chain variable region polypeptide, antibody or antibody fragment further comprises at least a C_{H}1 domain.

In a still further aspect, the variable region light chain, antibody or antibody fragment of the anti-TIGIT antibody described above and below, further comprises a C_{L} domain.

In a still further aspect, the antibody further comprises a C_{H}1, a C_{H}2, a C_{H}3 and a C_{L} domain.

In a still further aspect, the antibody further comprises a human or murine constant region.

In an embodiment, the human constant region is selected from the group consisting of IgG1, IgG2, IgG3, IgG4.

In a still further aspect, the anti-TIGIT antibodies of the invention are fully human antibodies.

In a still further aspect, the invention provides for an isolated anti-TIGIT antibody comprising a heavy chain and a light chain variable region sequence, wherein:

(a) the heavy chain sequence has at least 85% sequence identity to the heavy chain sequence:

(SEQ ID NO: 16)
QVQLVQSGSELKKPGASVKVSCKASGYTFTSYPMNVRQ
APGQGLEWMGWINTNTGNPTYAQGFTGRFVFSLDTSVS
TAYLQISSLKAEDTAVYYCARVGGYSVDEYAFDVWGQG
TLVTVSS,

and

(b) the light chain sequence has at least 85% sequence identity to the light chain sequence:

(SEQ ID NO: 17)
DIQLTQSPSFLSASVGDRVTITCRASQGISSYLAWYQQ
KPGKAPKLLIYAASTLQSGVPSRFSGSGSGTEFTLTISS
LQPEDFATYYCQQLSSYPTFGGGTKVEIK.

In a specific aspect, the sequence identity is 86%, 87%, 88%, 89%, 90%, 91%, 92%, 93%, 94%, 95%, 96%, 97%, 98%, 99% or 100%.

In a more specific aspect, the sequence identity is 100%.

In a very specific aspect, the anti-TIGIT antibody is a fully human IgG1 antibody, and the heavy and light chain variable region sequences further comprise human constant region sequences to yield the following full length heavy and light chain sequences:

Heavy chain:
(SEQ ID NO: 18)
QVQLVQSGSELKKPGASVKVSCKASGYTFTSYPMN
WWRQAPGQGLEWMGWINTNTGNPTYAQGFTGRFVF
SLDTSVSTAYLQISSLKAEDTAVYYCARVGGYSVD
EYAFDVWGQGTLVTVSSASTKGPSVFPLAPSSKST
SGGTAALGCLVKDYFPEPVTVSWNSGALTSGVHTF
PAVLQSSGLYSLSSVVTVPSSSLGTQTYICNVNHK
PSNTKVDKRVEPKSCDKTHTCPPCPAPELLGGPSV
FLFPPKPKDTLMISRTPEVTCVVVDVSHEDPEVKF
NWYVDGVEVHNAKTKPREEQYNSTYRVVSVLTVLH
QDWLNGKEYKCKVSNKALPAPIEKTISKAKGQPRE
PQVYTLPPSREEMTKNQVSLTCLVKGFYPSDIAVE
WESNGQPENNYKTTPPVLDSDGSFFLYSKLTVDKS
RWQQGNVFSCSVMHEALHNHYTQKSLSLSPG.

In an embodiment, depending on the expression system, the heavy chain may comprise a terminal K (lysine) residue.

Light chain:
(SEQ ID NO: 19)
DIQLTQSPSFLSASVGDRVTITCRASQGISSYLAW
YQQKPGKAPKLLIYAASTLQSGVPSRFSGSGSGTE
FTLTISSLQPEDFATYYCQQLSSYPTFGGGTKVEI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC.

which antibody is hereinbelow referred to as 3963H03-12, or in brief, H03-12. All specific (i.e. not including variables) sequences described above are those of antibody H03-12.

In other very specific aspects, the invention provides for anti-TIGIT antibodies referred to as 3964A06, 3965D08, 3966C11, 7728B03 and 7729G05, respectively, or in brief, A06, D08, C11, B03 and G05, whose sequences are described hereinbelow.

In an embodiment, the antibody is capable of binding to human and cynomolgus monkey TIGIT.

In more specific embodiment the antibody is capable of blocking the interaction between human or cynomolgus monkey TIGIT and the respective human or cynomolgus monkey CD155/PVR receptors.

In another embodiment, the antibody binds to human TIGIT with a K_{D} of 10×10⁻⁹ M or less, preferably with a K_{D} of 6×10⁻⁹ M or less, and even more preferred with a K_{D} of 4×10⁻⁹ M or less.

In another aspect, the invention concerns an isolated anti-TIGIT antibody (H03-12) or antigen binding fragment thereof which binds to a functional epitope comprising residues Q53, T55, Y113 and P114 of human TIGIT.

In an embodiment, the antibody (H03-12) binds to a functional epitope further comprising Q56, N70, and H111 of human TIGIT.

In a further aspect, the antibody (H03-12) binds to a conformational epitope comprising T51, Q53, T55, H111, T112, Y113, P114, and G116 of human TIGIT.

In an embodiment, the conformational epitope comprises T51, A52, Q53, T55, Q56, A71, D72, H111, T112, Y113, P114, G116 and T117 of human TIGIT.

In another embodiment, the antibody (H03-12) binds to a conformational epitope comprising T51, A52, Q53, T55, Q56, N70, D72, H111, T112, Y113, P114, and G116 of human TIGIT.

In another embodiment, the antibody (A06) binds to a conformational epitope comprising T51, A52, Q53, T55, Q56, N70, A71, D72, H111, T112, Y113, P114, G116 and T117 of human TIGIT.

In another embodiment, the antibody (C11) binds to a conformational epitope comprising T51, A52, Q53, T55, Q56, N70, A71, D72, H111, T112, Y113, P114, and G116 of human TIGIT.

In another embodiment, the antibody (B03) binds to a conformational epitope comprising T51, A52, Q53, T55, Q56, N70, A71, D72, H111, T112, Y113, P114, D115, G116 and T117 of human TIGIT.

In another embodiment, the antibody (G05) binds to a conformational epitope comprising M23, T51, Q53, V54, T55, Q56, N70, A71, H111, T112, Y113, P114, D115, G116, and T117 of human TIGIT.

In a further aspect, the invention is related to an anti-TIGIT antibody, or antigen binding fragment thereof, which cross-competes for binding to TIGIT with an antibody according to the invention as described herein.

In a further aspect, the invention provides for pharmaceutical compositions comprising an above described anti-TIGIT antibody, or antigen binding fragment thereof, in combination with at least one pharmaceutically acceptable carrier.

In a further aspect, the invention provides for an isolated nucleic acid encoding a polypeptide, or antibody light chain or heavy chain, or variable region sequences of an anti-TIGIT antibody, or antigen binding fragment thereof, as described herein.

In an embodiment, the isolated nucleic acid encoding the said heavy chain has the following sequence:

(SEQ ID NO: 20)
ATGGAAACAGACACCCTGCTGCTGTGGGTGCTGCT
GCTGTGGGTGCCCGGCTCCACAGGCCAGGTGCAGC
TGGTGCAGTCCGGCTCCGAGCTGAAGAAACCCGGC
GCCTCCGTGAAGGTGTCCTGCAAGGCCTCCGGCTA
CACCTTCACCTCCTACCCCATGAACTGGGTGAGGC
AGGCTCCTGGCCAGGGACTGGAGTGGATGGGCTGG
ATCAACACCAACACCGGCAACCCTACCTACGCCCA
GGGCTTCACCGGCAGGTTCGTGTTCTCCCTGGACA
CCAGCGTGTCCACCGCCTACCTGCAGATCTCCTCC
CTGAAGGCCGAGGACACCGCCGTGTACTACTGCGC
CAGGGTGGGAGGCTACTCCGTGGACGAGTACGCCT
TCGACGTGTGGGGCCAGGGCACCCTGGTGACCGTG
TCCTCCGCTAGCACCAAGGGCCCATCGGTCTTCCC
CCTGGCACCCTCCTCCAAGAGCACCTCTGGGGGCA
CAGCGGCCCTGGGCTGCCTGGTCAAGGACTACTTC
CCCGAACCGGTGACGGTGTCGTGGAACTCAGGCGC
CCTGACCAGCGGCGTGCACACCTTCCCGGCTGTCC
TACAGTCCTCAGGACTCTACTCCCTCAGCAGCGTG
GTGACCGTGCCCTCCAGCAGCTTGGGCACCCAGAC
CTACATCTGCAACGTGAATCACAAGCCCAGCAACA
CCAAGGTGGACAAGAGAGTTGAGCCCAAATCTTGT
GACAAAACTCACACATGCCCACCGTGCCCAGCACC
TGAACTCCTGGGGGGACCGTCAGTCTTCCTCTTCC
CCCCAAAACCCAAGGACACCCTCATGATCTCCCGG
ACCCCTGAGGTCACATGCGTGGTGGTGGACGTGAG
CCACGAAGACCCTGAGGTCAAGTTCAACTGGTACG
TGGACGGCGTGGAGGTGCATAATGCCAAGACAAAG
CCGCGGGAGGAGCAGTACAACAGCACGTACCGTGT
GGTCAGCGTCCTCACCGTCCTGCACCAGGACTGGC
TGAATGGCAAGGAGTACAAGTGCAAGGTCTCCAAC
AAAGCCCTCCCAGCCCCCATCGAGAAAACCATCTC
CAAAGCCAAAGGGCAGCCCCGAGAACCACAGGTGT
ACACCCTGCCCCCATCACGGGAGGAGATGACCAAG
AACCAGGTCAGCCTGACCTGCCTGGTCAAAGGCTT
CTATCCCAGCGACATCGCCGTGGAGTGGGAGAGCA
ATGGGCAGCCGGAGAACAACTACAAGACCACGCCT
CCCGTGCTGGACTCCGACGGCTCCTTCTTCCTCTA
TAGCAAGCTCACCGTGGACAAGAGCAGGTGGCAGC
AGGGGAACGTCTTCTCATGCTCCGTGATGCATGAG
GCTCTGCACAACCACTACACGCAGAAGAGCCTCTC
CCTGTCCCCGGGT.

In an embodiment, the isolated nucleic acid encoding the said light chain has the following sequence:

(SEQ ID NO: 21)
ATGAGGGCCCTGCTGGCTAGACTGCTGCTGTGCGT
GCTGGTCGTGTCCGACAGCAAGGGCGACATCCAGC
TGACCCAGTCCCCCTCCTTCCTGTCCGCTTCCGTG
GGCGACAGGGTGACCATCACTTGTCGTGCCTCCCA
GGGCATCTCCTCCTACCTGGCCTGGTACCAGCAGA
AGCCCGGCAAGGCCCCCAAGCTGCTGATCTACGCC
GCTTCCACACTGCAGTCCGGCGTGCCCTCCAGGTT
TTCCGGATCCGGCTCCGGCACCGAGTTCACCCTGA
CCATCTCCTCCCTGCAGCCCGAGGACTTCGCCACC
TACTACTGCCAGCAGCTGTCCTCCTACCCCACCTT
CGGCGGCGGCACAAAGGTGGAGATCAAGCGTACGG
TGGCTGCACCATCTGTCTTCATCTTCCCGCCATCT
GATGAGCAGTTGAAATCTGGAACTGCCTCTGTTGT
GTGCCTGCTGAATAACTTCTATCCCAGAGAGGCCA
AAGTACAGTGGAAGGTGGATAACGCCCTCCAATCG
GGTAACTCCCAGGAGAGTGTCACAGAGCAGGACAG
CAAGGACAGCACCTACAGCCTCAGCAGCACCCTGA
CGCTGAGCAAAGCAGACTACGAGAAACACAAAGTC
TACGCCTGCGAAGTCACCCATCAGGGCCTGAGCTC
GCCCGTCACAAAGAGCTTCAACAGGGGAGAGTGT.

In another aspect, the invention provides for a vector, which is suitable for the expression of one or more of the said nucleic acids.

In a further aspect, the invention provides for a host cell, comprising the said vector, suitable for the expression of the nucleic acid, and for delivering a mature, correctly folded polypeptide, or antibody light chain or heavy chain, or variable region sequences of an anti-TIGIT antibody, or antigen binding fragment thereof, as described herein.

In an embodiment, the host cell is a eukaryotic cell or a prokaryotic cell.

In a specific embodiment, the eukaryotic cell is a mammalian cell, such as Chinese Hamster Ovary (CHO).

In a more specific embodiment, the CHO cell is CHO-K1SV.

In another aspect, the invention provides for a process of making an anti-TIGIT antibody or antigen binding fragment thereof, comprising culturing a host cell containing nucleic acid encoding any of the previously described TIGIT antibodies or antigen-binding fragment in a form suitable for expression, under conditions suitable to produce such antibody or fragment, and recovering the antibody or fragment.

In yet another aspect, the invention is directed to engineered anti-TIGIT antibodies, or engineered fragments thereof, which are fused directly or via a linker molecule to therapeutic agents, such as cytokines or growth factors. Such engineered antibodies or engineered antibody fragments may also be used in tumor therapy and immune system related diseases. Antibody fusion proteins, especially immunocytokines, are well known in the art. The fusion partner can be bound to the N-terminus of the antibody or antibody fragment or to its C-terminus.

In a still further aspect, the invention provides for a method of treating cancer comprising administering to a subject in need thereof an effective amount of an anti-TIGIT antibody as herein disclosed, or a pharmaceutical composition as herein disclosed.

In an embodiment, the cancer is selected from the group consisting of: breast, lung, colon, ovarian, melanoma, bladder, kidney, liver, salivary, stomach, gliomas, thyroid, thymic, epithelial, head and neck cancers, gastric and pancreatic cancer.

In a still further aspect, the invention provides for a method of enhancing T-cell function comprising administering an effective amount of any of the above described anti-TIGIT antibodies or compositions.

In an embodiment, the anti-TIGIT antibody or composition renders dysfunctional T-cells non-dysfunctional.

In a still further embodiment, the invention provides for a method of treating a T-cell dysfunctional disorder comprising administering a therapeutically effective amount of any of the above described anti-TIGIT antibodies or compositions.

In one specific aspect, the T-cell dysfunctional disorder is tumor immunity.

In a still further specific aspect, the tumor immunity results from a cancer selected from the group consisting of breast, lung, colon, ovarian, melanoma, bladder, kidney, liver, salivary, stomach, gliomas, thyroid, thymic, epithelial, head and neck cancers, gastric, and pancreatic cancer.

In an even more specific aspect, the tumor immunity results from a cancer selected from the group consisting of lung, head and neck, colon, bladder and kidney cancer.

Therefore, in another aspect the methods of this invention may find use in treating conditions where enhanced immunogenicity is desired, such as increasing tumor immunogenicity, for the treatment of cancer, which cancer may be at early stage or at late stage and/or metastatic.

In some embodiments of the methods of the present invention, some cancers have elevated levels of tumor infiltrating lymphocytes (TILs) which may refer to the presence of T cells within the cancer tissue. It is known in the art that T cell infiltration may be associated with improved clinical outcome in certain cancers (see, e.g., Zhang et al, N. Engl. J. Med. 348(3):203-213 (2003)). However, in the tumor environment, TILs also include exhausted T cells (e.g. CD8+ T cells) and suppressive T cells (e.g. regulatory T cells) expressing high levels of inhibitory co-receptors such as PD-1, TIGIT, TIM3, LAG3, and lacking the capacity to produce effector cytokines. It is expected that an anti-TIGIT antibody with ADCC potential will block TIGIT interaction to prevent and/or rescue T-cells from exhaustion, and to reduce suppressive T cells.

In some embodiments of the methods of the present invention, the individual has a T cell dysfunctional disorder which, in some embodiments, is characterized by T cell anergy or decreased ability to secrete cytokines, proliferate or execute cytolytic activity. In other embodiments the T cell dysfunctional disorder is characterized by T cell exhaustion. In some embodiments the T cells are CD4+ and CD8+ T cells.

Equivalent to the above mentioned methods of enhancing T-cell function, treating a T-cell dysfunctional disorder, or treating cancer, the invention relates likewise to the use of an anti-TIGIT antibody or composition as described above and below for the manufacture of a medicament for enhancing T-cell function, treating a T-cell dysfunctional disorder or treating cancer, or to an anti-TIGIT antibody or composition for use in the enhancement of T-cell function, or treatment of a T-cell dysfunctional disorder or cancer.

In another aspect, provided herein are methods for increasing, enhancing or stimulating an immune response or function in an individual having cancer comprising administering to the individual an effective amount of an anti-TIGIT antibody and an anti-cancer agent and/or an anti-cancer therapy.

In another aspect, provided herein are methods for treating or delaying progression of tumor immunity or cancer, or reducing or inhibiting cancer relapse in an individual comprising administering to the individual an effective amount of an anti-TIGIT antibody and an anti-cancer agent and/or an anti-cancer therapy.

In certain embodiments, the methods comprise administering to the individual an effective amount of an anti-TIGIT antibody, and/or an anti-cancer agent, and/or an anti-cancer therapy.

In certain embodiments, the anticancer therapy is selected from the group consisting of radiation therapy, surgery, chemotherapy, gene therapy, DNA therapy, viral therapy, RNA therapy, immunotherapy, bone marrow transplantation, nanotherapy, monoclonal antibody therapy, adjuvant therapy, neoadjuvant therapy, hormonal therapy, angiogenesis inhibition, palliative care and combinations thereof.

In certain embodiments that may be combined with any of the preceding embodiments, the anti-cancer agent is selected from the group consisting of a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof.

In certain embodiments, the chemotherapeutic or growth inhibitory agent is selected from the group consisting of an alkylating agent, an anthracycline, an anti-hormonal agent, an aromatase inhibitor, an anti-androgen, a protein kinase inhibitor, a lipid kinase inhibitor, an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof.

In certain embodiments that may be combined with any of the preceding embodiments, the targeted therapeutic agent is selected from the group consisting of a B-raf inhibitor, a MEK inhibitor, a K-ras inhibitor, a c-Met inhibitor, an Alk inhibitor, a phosphatidylinositol 3-kinase inhibitor, an Akt inhibitor, a p70S6K inhibitor, a BTK inhibitor, an mTOR inhibitor, a dual phosphatidylinositol 3-kinase/mTOR inhibitor, and combinations thereof.

In certain embodiments that may be combined with any of the preceding embodiments, the targeted therapeutic agent is an antibody, or antigen-binding fragment thereof, or antibody fusion protein, selected from the group consisting of alemtuzumab, apolizumab, atezolizumab, avelumab, bevacizumab, blinatumomab, catumaxomab, cemiplimab, cetuximab, daratumumab, durvalumab, eculizumab, elotuzumab, emicizumab, epratuzumab, gemtuzumab-ozogamincin, ibritumomab-tiuxetan, inotuzumab-ozogamicin, ipilimumab, mogamulizumab, necitumumab, nivolumab, obinutuzumab, ofatumumab, olaratumab, oregovomab, pantitumumab, pembrolizumab, pertuzumab, ramucirumab, retuximab, rovalpituzumab-teserine, siltuximab, tremelimumab, tositumomab, trastuzumab, zanolimumab, anti-IL-12, and anti-IL-17. In a more specific embodiment, the at least one therapeutic agent is avelumab.

In certain embodiments that may be combined with any of the preceding embodiments, the antibody or antigen-binding fragment thereof specifically binds to a target selected from the group consisting of PD-1, PD-L1, CTLA-4, CD52, VEGF-A, EGFR, CD20, HER2, HLA-DRB, CD62L, IL-6R, amyloid beta, CD44, CanAg, CD4, TNF alpha, IL-2, CD25, complement C5, CDI la, CD22, CD18, respiratory syncytial virus F, interferon gamma, CD33, CEACAM5, IL-5, integrin alpha 4, IgE, IL-4, IL-5, CD154, FAP, CD2, MUC-1, AFP, integrin alpha-v-beta-3, IL6R, CD40L, EpCAM, Shiga-like toxin II, IL-12, IL-23, IL-17, and CD3.

In an embodiment, the anti-TIGIT antibody is administered before the anti-cancer agent or anti-cancer therapy. In another embodiment, the anti-TIGIT antibody is administered simultaneously with the anti-cancer agent or anti-cancer therapy. In yet another embodiment the anti-TIGIT antibody is administered after the anti-cancer agent or anticancer therapy.

Another aspect of the invention relates to the use of antibody dependent cell-mediated cytotoxicity (ADCC) of an anti-TIGIT antibody disclosed herein or composition in the treatment of cancer. Therefore, the invention pertains to a method of treating cancer comprising administering to a subject in need thereof an effective amount of an anti-TIGIT antibody which induces antibody dependent cell-mediated cytotoxicity (ADCC).

In another aspect, the antibody or composition treats or prevents a symptom of persistent infection, such as viral infection, e.g. by human immunodeficiency virus (HIV), herpes virus, Eppstein-Barr virus or human papilloma virus.

In a still further aspect, the invention provides a kit of parts comprising a pharmaceutical composition disclosed herein, and a package insert indicating use for the treatment of a T-cell dysfunctional disorder and/or cancer in an individual.

In a still further aspect, the present disclosure provides a kit comprising a pharmaceutical composition disclosed herein, an anti-cancer agent, and a package insert comprising instructions for using the anti-cancer agent in combination with an anti-TIGIT antibody to treat a T-cell dysfunctional disorder and/or cancer in an individual.

### Definitions

Antibody Related Definitions

The term “antibody” includes monoclonal antibodies (including full length antibodies which have an immunoglobulin Fc region), antibody compositions with poly-epitopic specificity, multi-specific antibodies (e.g., bispecific antibodies, diabodies, and single-chain molecules, as well as antibody fragments (e.g., Fab, F(ab′)2, and Fv). The term “immunoglobulin” (Ig) is used interchangeably with “antibody” herein. The basic 4-chain antibody unit is a hetero-tetrameric glycoprotein composed of two identical light (L) chains and two identical heavy (H) chains. An IgM antibody consists of 5 of the basic hetero-tetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the alpha and gamma heavy chain isotypes and four CH domains for mu and epsilon heavy chain isotypes. Each L chain has at the N-terminus, a variable domain (VL) followed by a constant domain at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton and Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated alpha, delta, epsilon, gamma and mu, respectively. The gamma and alpha classes are further divided into subclasses based on relatively minor differences in the CH sequence and function, e.g., humans express the following subclasses: IgGI, IgG2, IgG3, IgG4, IgA1 and IgA2.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of an antibody for its antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al, Immunity 13:37-45 (2000); Johnson and Wu, in Methods in Molecular Biology 248: 1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al, Nature 363:446-448 (1993); Sheriff et al, Nature Struct. Biol. 3:733-736 (1996). A number of HVR delineations are in use. The ImMunGeneTics (IMGT) unique Lefranc numbering (IMGT numbering) (Lefranc, M.-P. et al. Dev. Comp. Immunol., 27, 55-77 (2003)) takes into account sequence conservation, structural data from X-ray diffraction studies, and the characterization of the hypervariable loops in order to define the FR and HVR. The Kabat Complementarity Determining Regions (CDRs) are based on sequence variability and are also commonly used (Kabat et al, Sequences of Proteins of Immunological Interest, 5th Ed. Public Health Service, National Institutes of Health, Bethesda, Md. (1991)). Chothia refers instead to the location of the structural loops (Chothia and Lesk, I. Mol. Biol. 196:901-917 (1987)). The AbM HVRs represent a compromise between the Kabat HVRs and Chothia structural loops and are used by Oxford Molecular's AbM antibody modeling software. The “contact” HVRs are based on an analysis of the available complex crystal structures.

The residues from each of these HVRs are noted below.

| | | | | | IMGT | IMGT |
|---|---|---|---|---|---|---|
| | | | | | (Kabat | (IMGT |
| Loop | Kabat | AbM | Chothia | Contact | Numbering) | numbering) |
| L1 | L24-L34 | L24-L34 | L26-L32 | L30-L36 | L27-L32 | 27-38 |
| L2 | L50-L56 | L50-L56 | L50-L52 | L46-L55 | L50-L52 | 56-65 |
| L3 | L89-L97 | L89-L97 | L91-L96 | L89-L96 | L89-L97 | 105-117 |
| H1 | H31-H35B | H26-H35B | H26-H32 | H30-H35 | H26-H33 | 27-38 |
| | | | | (Kabat | | |
| | | | | numbering) | | |
| H1 | H31-H35 | H26-H35B | H26-H32 | H30-H35 | H26-H33 | 27-38 |
| | | | | (Chothia | | |
| | | | | numbering) | | |
| H2 | H50-H65 | H50-H58 | H53-H55 | H47-H58 | H51-H57 | 56-65 |
| H3 | H95-H102 | H95-H102 | H96-H101 | H93-H101 | H93-H102 | 105-117 |

HVRs may comprise “extended HVRs” as follows: 24-40 (LI), 56-69 (L2) and 105-117 (L3) in the VL and 24-40 (HI), 55-74 (H2) and 105-117 (H3) in the VH. The variable domain residues are numbered according to Lefranc et al, supra, for each of these definitions. The expression “variable-domain residue-numbering using IMGT definitions” or “amino-acid-position numbering as in IMGT” and variations thereof, refers to the numbering system used for antibody variable domains in Lefranc et al., supra. Using this numbering system, the actual linear amino acid sequence may contain fewer or additional amino acids corresponding to a shortening of, or insertion into, a FR or HVR of the variable domain. For example, a heavy-chain variable domain may include inserted residues (e.g. residues 111.1 and 112.1, etc. according to Lefranc) after heavy-chain HVR residue 111 or before heavy chain HVR 112. The IMGT numbering of residues may be determined for a given antibody by alignment at regions of homology of the sequence of the antibody with a “standard” IMGT numbered sequence.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

A “VH subgroup I consensus framework” comprises the consensus sequence obtained from the amino acid sequences in variable heavy subgroup I of Kabat et al, supra.

A “VL kappa I consensus framework” comprises the consensus sequence obtained from the amino acid sequences in variable light kappa subgroup I of Kabat et al, supra.
The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Unless indicated otherwise herein, the numbering of the residues in an immunoglobulin heavy chain constant region is that of the EU index as in Kabat et al, supra. The “EU index as in Kabat” refers to the residue numbering of the human IgG1 EU antibody.—The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgGI, IgG2, IgG3 and IgG4. [0068]“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain, (see M. Daeron, Annu. Rev. Immunol. 15:203-234 (1997). FcRs are reviewed in Ravetch and Kinet, Annu. Rev. Immunol. 9: 457-92 (1991); Capel et al, Immunomethods 4: 25-34 (1994); and de Haas et al, J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al, J. Immunol. 117: 587 (1976) and Kim et al, J. Immunol. 24: 249 (1994). Methods of measuring binding to FcRn are known (see, e.g., Ghetie and Ward, Immunol. Today 18: (12): 592-8 (1997); Ghetie et al, Nature Biotechnology 15 (7): 637-40 (1997); Hinton et al, J. Biol. Chem. 279 (8): 6213-6 (2004); WO 2004/92219 (Hinton et al). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al, J. Biol. Chem. 9(2): 6591-6604 (2001).

The “Fc fragment” comprises the carboxy-terminal portions of both H chains held together by disulfides. The effector functions of antibodies are determined by sequences in the Fc region, the region which is also recognized by Fc receptors (FcR) found on certain types of cells. “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen.

An “amino-acid modification” at a specified position, e.g. of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)2 and Fv fragments; diabodies; linear antibodies (see U.S. Pat. No. 5,641,870, Example 2; Zapata et al, Protein Eng. 8(10): 1057-1062 [1995]); single-chain antibody molecules and multi-specific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994). “Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the VH and VL domains such that inter-chain but not intra-chain pairing of the V domains is achieved, thereby resulting in a bivalent fragment, i. e., a fragment having two antigen-binding sites. Bispecific diabodies are heterodimers of two “crossover” sFv fragments in which the VH and VL domains of the two antibodies are present on different polypeptide chains. Diabodies are described in greater detail in, for example, EP 404,097; WO 93/11161; Hollinger et al, Proc. Natl. Acad. Sci. USA 90: 6444-6448 (1993). The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma and Immunol. 1: 105-115 (1998); Harris, Biochem. Soc. Transactions 23: 1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, I. Mol. Biol., 227:381 (1991); Marks et al, J. Mol. Biol, 222:581 (1991). Also available for the preparation of human monoclonal antibodies are methods described in Dijk and van de Winkel, Curr. Opin. Pharmacol, 5: 368-74 (2001). Human antibodies can be prepared by administering the antigen to a transgenic animal that has been genetically modified to produce partial or full human antibodies in response to antigenic challenge, but whose endogenous loci have been disabled, e.g., OmniAb therapeutic antibody platforms (Ligand Pharmaceuticals), immunized xenomice (see, e.g., U.S. Pat. Nos. 6,075,181 and 6,150,584 regarding Xenomouse technology), etc. See also, for example, Li et al, Proc. Natl. Acad. Set USA, 103:3557-3562 (2006) regarding human antibodies generated via a human B-cell hybridoma technology.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., Jakobovits et al., Proc. Natl. Acad. Sci. USA 90: 2551 (1993); Jakobovits et al., Nature 362: 255-258 (1993); Bruggemann et al., Year in Immunol. 7:33 (1993); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al, Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169: 147-155 (1995); Yelton et al. J. Immunol. 155: 1994-2004 (1995); Jackson et al, J. Immunol. 154(7):3310-9 (1995); and Hawkins et al, J. Mol. Biol. 226:889-896 (1992).

As used herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG1, IgG2, IgG-3, or IgG-4 subtypes, IgA (including IgA1 and IgA2), IgE, IgD or IgM. The Ig fusions preferably include the substitution of a domain of a polypeptide or antibody described herein in the place of at least one variable region within an Ig molecule. In a particularly preferred embodiment, the immunoglobulin fusion includes the hinge, CH2 and CH3, or the hinge, CH1, CH2 and CH3 regions of an IgGI molecule. For the production of immunoglobulin fusions see also U.S. Pat. No. 5,428,130 issued Jun. 27, 1995. Immunoadhesin combinations of Ig Fc and ECD of cell surface receptors are sometimes termed soluble receptors.

A “blocking” antibody or an “antagonist” antibody is one that inhibits or reduces a biological activity of the antigen it binds. In some embodiments, blocking antibodies or antagonist antibodies substantially or completely inhibit the expression or biological activity of the antigen. For example, the anti-TIGIT antibodies or antigen-binding fragments thereof of the present disclosure may inhibit TIGIT expression, block the interaction of TIGIT with PVR, block the interaction of TIGIT with PVRL2, block the interaction of TIGIT with PVRL3, inhibit and/or block the intracellular signaling mediated by TIGIT binding to PVR, inhibit and/or block the intracellular signaling mediated by TIGIT binding to PVRL2, and/or inhibit and/or block the intracellular signaling mediated by TIGIT binding to PVRL3.

An “agonist” or activating antibody is one that enhances or initiates signaling by the antigen to which it binds. In some embodiments, agonist antibodies cause or activate signaling without the presence of the natural ligand.

The terms “cross-compete”, “cross-competition”, “cross-block”, “cross-blocked” and “cross-blocking” are used interchangeably herein to mean the ability of an antibody or fragment thereof to interfere with the binding directly or indirectly through allosteric modulation of the antibodies of the invention to their target antigen. The extent to which an antibody or fragment thereof is able to interfere with the binding of another to the target, and therefore whether it can be said to cross-block or cross-compete according to the invention, can be determined using competition binding assays. One particularly suitable quantitative cross-competition assay uses a FACS- or an AlphaScreen-based approach to measure competition between the labelled (e.g. His tagged, biotinylated or radioactive labelled) an antibody or fragment thereof and the other an antibody or fragment thereof in terms of their binding to the target. In general, a cross-competing antibody or fragment thereof is for example one which will bind to the target in the cross-competition assay such that, during the assay and in the presence of a second antibody or fragment thereof, the recorded displacement of the immunoglobulin single variable domain or polypeptide according to the invention is up to 100% (e.g. in FACS based competition assay) of the maximum theoretical displacement (e.g. displacement by cold (e.g. unlabelled) antibody or fragment thereof that needs to be cross-blocked) by the to be tested potentially cross-blocking antibody or fragment thereof that is present in a given amount. Preferably, cross-competing antibodies or fragments thereof have a recorded displacement that is between 10% and 100%, more preferred between 50% to 100%. “Bispecific antibodies” are monoclonal, preferably human or humanized, antibodies that have binding specificities for at least two different antigens. Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)).

“Binding affinity” generally refers to the strength of the total sum of non-covalent interactions between a single binding site of a molecule (e.g., of an antibody) and its binding partner (e.g., an antigen). Unless indicated otherwise, as used herein, “binding affinity”, “bind to”, “binds to” or “binding to” refers to intrinsic binding affinity that reflects a 1 to 1 interaction between members of a binding pair (e.g., antibody Fab fragment and antigen). The affinity of a molecule X for its partner Y can generally be represented by the dissociation constant (KD). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity, i.e. binding strength are described in the following.

The “KD” or “KD value” according to this invention can be measured by a radiolabeled antigen binding assay (RIA) performed with the Fab version of the antibody and antigen molecule, or by using surface-plasmon resonance assays using a BIACORE instrument (BIAcore, Inc., Piscataway, N.J.).

The term “functional epitope” as used herein refers to amino acid residues of an antigen that contribute energetically to the binding of an antibody, i.e. forming an “energetic epitope”. Mutation of any one of the energetically contributing residues of the antigen to alanine will disrupt the binding of the antibody, without disrupting folding of the antigen, such that the relative KD ratio (KD mutant TIGIT/KD wild type TIGIT) of the antibody will be greater than 3.2, which is equivalent to 0.7 kcal/mol in terms of ΔΔG.

The term “conformational epitope” as used herein refers to amino acid residues of the TIGIT antigen that come together on the surface when the polypeptide chain folds to form the native protein, and are within 3.8 angstroms of amino acid residues in the bound Fab in the co-crystal structure. The conformation epitope contains, but is not limited to, the functional epitope.

Immune System Related Definitions

“Immunogenicity” refers to the ability of a substance to provoke an immune response. Tumors are immunogenic and enhancing tumor immunogenicity aids in the clearance of the tumor cells by the immune response. Examples of enhancing tumor immunogenicity include but not limited to treatment with a inhibitors of immune modulatory receptors.

The term “vaccine” as used herein includes any nonpathogenic immunogen that, when inoculated into a host, induces protective immunity against a specific pathogen. Vaccines can take many forms. Vaccines can be whole organisms that share important antigens with the pathogen, but are not pathogenic themselves (e.g., cowpox). Vaccines can also be prepared from killed (e.g., Salk polio vaccine) or attenuated (lost ability to produce disease—e.g., Sabin polio vaccine). Vaccines can also be prepared from purified macromolecules isolated from the pathogenic organism.

“Enhancing T-cell function” means to induce, cause or stimulate a T-cell to have a sustained or amplified biological function, or renew or reactivate exhausted or inactive T-cells. Examples of enhancing T-cell function include: increased secretion of gamma-interferon from CD8+ T-cells, increased proliferation, increased antigen responsiveness (e.g., viral, pathogen, or tumor clearance) relative to such levels before the intervention. In one embodiment, the level of enhancement is at least 50 percent, alternatively 60 percent, 70 percent, 80 percent, 90 percent, 100 percent, 120 percent, 150 percent, 200 percent. The manner of measuring this enhancement is known to one of ordinary skill in the art.

A “T-cell dysfunctional disorder” is a disorder or condition of T-cells characterized by decreased responsiveness to antigenic stimulation (e.g., against a tumor expressing an immunogen). For exampöe, a T-cell dysfunctional disorder can be characterized by T-cells which are anergic or have decreased ability to secrete cytokines, proliferate, or execute cytolytic activity. The decreased responsiveness may result in ineffective control of a tumor expressing an immunogen. Examples of T cell dysfunctional disorders characterized by T-cell dysfunction include tumor immunity and cancer.

The term “dysfunction” in the context of immune dysfunction, refers to a state of reduced immune responsiveness to antigenic stimulation. The term includes the common elements of both exhaustion and/or anergy in which antigen recognition may occur, but the ensuing immune response is ineffective to control infection or tumor growth.

The term “dysfunctional”, as used herein, also includes refractory or unresponsive to antigen recognition, specifically, impaired capacity to translate antigen recognition into down-stream T-cell effector functions, such as proliferation, cytokine production (e.g., IL-2) and/or target cell killing.

The term “anergy” refers to the state of unresponsiveness to antigen stimulation resulting from incomplete or insufficient signals delivered through the T-cell receptor (e.g. increase in intracellular Ca+2 in the absence of ras-activation). T cell anergy can also result upon stimulation with antigen in the absence of co-stimulation, resulting in the cell becoming refractory to subsequent activation by the antigen even in the context of co-stimulation. The unresponsive state can often be overridden by the presence of Interleukin-2. Anergic T-cells do not undergo clonal expansion and/or acquire effector functions.

The term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors. Exhaustion can result from both extrinsic negative regulatory pathways (e.g., immunoregulatory cytokines) as well as cell intrinsic negative regulatory (costimulatory) pathways.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al, PNAS USA 95:652-656 (1998).

“Effector cells” are leukocytes which express one or more FcRs and perform effector functions. In one aspect, the effector cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils. The effector cells may be isolated from a native source, e.g., blood. Effector cells generally are lymphocytes associated with the effector phase, and function to produce cytokines (helper T cells), killing cells in infected with pathogens (cytotoxic T cells) or secreting antibodies (differentiated B cells).

An “autoimmune disorder” is a disease or disorder arising from and directed against an individual's own tissues or organs or a co-segregation or manifestation thereof or resulting condition therefrom. Autoimmune diseases can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease that can affect multiple organ systems (for example, systemic lupus erythematosus (SLE), rheumatoid arthritis (RA), polymyositis, etc.).

Cancer Related Definitions

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

As used herein, “cancer” and “cancerous” refer to or describe the physiological condition in mammals that is typically characterized by unregulated cell growth. Included in this definition are benign and malignant cancers as well as dormant tumors or micrometastases.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a duration of time.

As used herein, “complete response” or “CR” refers to disappearance of all target lesions; “partial response” or “PR” refers to at least a 30 percent decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20 percent increase in the SLD of target lesions, taking as reference the smallest SLD recorded since the treatment started or the presence of one or more new lesions.

Formulation and Drug Delivery Related Definitions

The term “‘pharmaceutical formulation” refers to a preparation that is in such form as to permit the biological activity of the active ingredient to be effective, and that contains no additional components that are unacceptably toxic to a subject to which the formulation would be administered. Such formulations are sterile.

A “sterile” formulation is aseptic or free from all living microorganisms and their spores.

The term “about” as used herein refers to the usual error range for the respective value readily known to the skilled person in this technical field.

The phrase “pharmaceutically acceptable” indicates that the substance or composition must be compatible chemically and/or toxicologically, with the other ingredients comprising a formulation, and/or the individual being treated therewith.

A “stable” formulation is one in which the protein therein essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. For example, the extent of aggregation during storage can be used as an indicator of protein stability. Thus, a “stable” formulation may be one wherein less than about 10% and preferably less than about 5% of the protein are present as an aggregate in the formulation.

A “reconstituted” formulation is one which has been prepared by dissolving a lyophilized protein or antibody formulation in a diluent such that the protein is dispersed throughout. The reconstituted formulation is suitable for administration (e.g. subcutaneous administration) to a patient to be treated with the protein of interest and, in certain embodiments of the invention, may be one which is suitable for parenteral or intravenous administration.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example.

“Pharmaceutically acceptable” buffers and salts include those derived from both acid and base addition salts of the above indicated acids and bases. Specific buffers and/or salts include histidine, succinate and acetate.

“Pharmaceutically acceptable carriers” as used herein refers to excipients that are nontoxic to the cell or individual being exposed thereto at the dosages and concentrations employed. Often the pharmaceutically acceptable carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, polyethylene glycol (PEG), and Pluronics.

A “package insert” refers to instructions customarily included in commercial packages of medicaments that contain information about the indications, usage, dosage, administration, contraindications, other medicaments to be combined with the packaged product, and/or warnings concerning the use of such medicaments, etc.

### Other Definitions

As used herein, the term “treatment” or “treating” refers to clinical intervention designed to alter the natural course of the individual or cell being treated during the course of clinical pathology, e.g., cancer or tumor immunity. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying progression of a disease” means to defer, hinder, slow, retard, stabilize, and/or postpone development of the disease (such as cancer or tumor immunity). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum concentration required to affect a measurable improvement or prevention of a disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be, or is achieved.

As used herein, “in combination with” refers to administration of one treatment modality in addition to another treatment modality. As such, “in combination with” refers to administration of one treatment modality before, during, or after administration of the other treatment modality to the individual. The term “in conjunction with” may be used interchangeably herein.

As used herein and in the appended claims, the singular forms “a,” “or,” and “the” include plural referents unless the context clearly dictates otherwise.

Reference to “about” a value or parameter herein includes (and describes) variations that are directed to that value or parameter per se. For example, description referring to “about X” includes description of “X”.

As used herein, the terms “individual” and “subject” may be used interchangeably and refer to a mammal, including, but not limited to, a human or non-human mammal, such as a bovine, equine, canine, ovine, or feline. Preferably, the individual or subject is a human. Patients are also individuals or subjects herein.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2 or ALIGN software. Those skilled in the art can determine appropriate parameters for measuring alignment, including any algorithms needed to achieve maximal alignment over the full length of the sequences being compared.

An “isolated” nucleic acid molecule encoding the polypeptides and antibodies herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., KD values). The difference between said two values is, for example, greater than about 10 percent, greater than about 20 percent, greater than about 30 percent, greater than about 40 percent, and/or greater than about 50 percent as a function of the value for the reference/comparator molecule.

The term “substantially similar’” or “substantially the same,’” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50 percent, less than about 40 percent, less than about 30 percent, less than about 20 percent, and/or less than about 10 percent as a function of the reference/comparator value.

A “fusion protein” and a “fusion polypeptide” refer to a polypeptide having two portions covalently linked together, where each of the portions is a polypeptide having a different property. The property may be a biological property, such as activity in vitro or in vivo. The property may also be simple chemical or physical property, such as binding to a target molecule, catalysis of a reaction, etc. The two portions may be linked directly by a single peptide bond or through a peptide linker but are in reading frame with each other. An example of an antibody fusion protein is bintrafusp alfa, a bifunctional molecule capable of binding to PD-L1 and TGFbeta.

The term “antagonist” is used in the broadest sense, and includes any molecule that partially or fully blocks, inhibits, or neutralizes a biological activity of a native polypeptide disclosed herein. In a similar manner, the term “agonist” is used in the broadest sense and includes any molecule that mimics a biological activity of a native polypeptide disclosed herein. Suitable agonist or antagonist molecules specifically include agonist or antagonist antibodies or antibody fragments, fragments or amino acid sequence variants of native polypeptides, peptides, antisense oligonucleotides, small organic molecules, etc.

Methods for identifying agonists or antagonists of a polypeptide may comprise contacting a polypeptide with a candidate agonist or antagonist molecule and measuring a detectable change in one or more biological activities normally associated with the polypeptide.

The terms “TIGIT antagonist” and “antagonist of TIGIT activity or TIGIT expression” are used interchangeably and refer to a compound that interferes with the normal functioning of TIGIT, either by decreasing transcription or translation of TIGIT-encoding nucleic acid, or by inhibiting or blocking TIGIT polypeptide activity, or both. Examples of TIGIT antagonists include, but are not limited to, antisense polynucleotides, interfering RNAs, catalytic RNAs, RNA-DNA chimeras, TIGIT-specific aptamers, anti-TIGIT antibodies, TIGIT-binding fragments of anti-TIGIT antibodies, TIGIT-binding small molecules, TIGIT-binding peptides, and other polypeptides that specifically bind TIGIT (including, but not limited to, TIGIT-binding fragments of one or more TIGIT ligands, optionally fused to one or more additional domains), such that the interaction between the TIGIT antagonist and TIGIT results in a reduction or cessation of TIGIT activity or expression. It will be understood by one of ordinary skill in the art that in some instances, a TIGIT antagonist may antagonize one TIGIT activity without affecting another TIGIT activity. For example, a desirable TIGIT antagonist for use in certain of the methods herein is a TIGIT antagonist that antagonizes TIGIT activity in response to one of PVR interaction, PVRL3 interaction, or PVRL2 interaction, e.g., without affecting or minimally affecting any of the other TIGIT interactions.

### DESCRIPTION OF THE FIGURES

FIG. 1

Graph showing the results of a competitive ELISA assessing the abilities of the anti-TIGIT antibodies to block the binding of TIGIT to CD155.

FIG. 2

A rendering of the crystal structures of anti-TIGIT Fabs bound to human TIGIT (gray), with the heavy chain in black and the light chain in light gray: A. 3963H03; B. 3966C11. C. 3964A06; D. 7729G05; E. 7728B03; F. 3963H03-12.

FIG. 3

A rendering of the crystal structure of Fab 3963H03-12 overlayed with a rendering of the crystal structure of TIGIT in complex with PVR (Protein Data Bank entry 3UDW) showing that 3963H03-12 overlaps the binding site of PVR on TIGIT. The surface of PVR is rendered in dark gray. The light chain of 3963H03-12 is shown in light gray and the heavy chain shown in dark gray.

FIG. 4

Human TIGIT ECD crystal structure with mutagenized residues contacting 3963H03 shown in sticks. The residues were colored according to the change of binding affinity upon mutating to alanine or glycine. (dark grey: >3 kcal/mol; medium grey >2 kcal/mol; light grey <0.7 kcal/mol).

FIG. 5

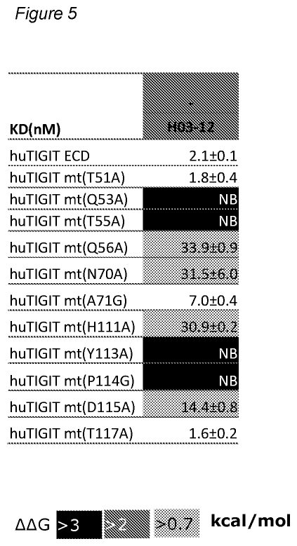

A summary of kinetic binding affinities of anti-TIGIT 3963H03-12 to TIGIT mutants is shown. Binding KDs highlighted by the loss of binding affinity upon mutation (ΔΔG). Positions where mutation causing significant binding energy loss was highlighted in three different shades, indicating the magnitude of loss. Binding affinities stronger than KD=0.1 nM were reported as <0.1 as it is beyond the instrument measuring range. NB indicates no binding. Standard deviation was reported if more than one experiment was performed.

FIG. 6

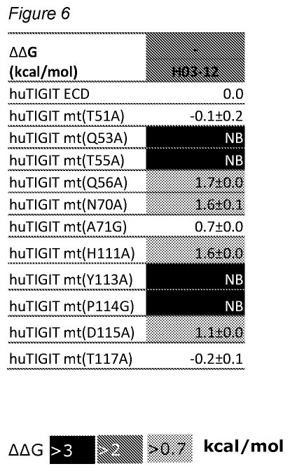

The change of binding affinity to TIGIT mutants, converted from kinetic affinity data, is shown for anti-TIGIT 3963H03-12. The binding affinity ΔG was calculated from KD with the equation ΔG=ln(KD)*RT. The change of binding affinity ΔΔG is the difference of binding affinity between mutant and parental TIGIT. If the KD for either variant is stronger than 0.1 nM, the ΔΔG is not calculated and it is indicated as ND (not determined).

FIG. 7

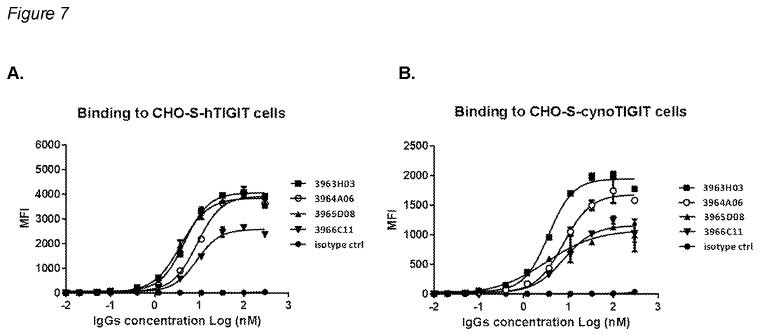

Graphs showing the results of cell based binding assays using CHO-S cells expressing the human TIGIT (A) or cynomolgus monkey TIGIT (B) extracellular domains. Anti-TIGIT antibodies were tested at varying concentrations and binding was measured by flow cytometry.

FIG. 8

Blockade of functional TIGIT/CD155 interaction. Blockade of TIGIT/CD155 interaction was measured in the presence of a range of concentrations of anti-TIGIT or isotype control antibody with cell-based Jurkat reporter assay (Promega CS198801). Sequence optimized 3963H03-12, parental 3963H03 and an isotype control were tested. Data was plotted and curve fitting and EC50 value calculations were performed with GraphPad Prism program. RLU, relative luciferase units.

FIG. 9

Graphs showing ADCC activities of anti-TIGIT antibodies 3963H03 and 3963H03-12 using as targets CHO-S cells expressing the human TIGIT extracellular domain.

FIG. 10

Graph showing the complement dependent cytotoxicity (CDC) of 3963H03-12, using as the targets ⁵¹Cr-labelled CHO-S cells expressing the human TIGIT extracellular domain

FIG. 11

Graph demonstrating that anti-TIGIT antibodies, A06, C11, D08, H03, enhanced IFNγ production in a T cell activation assay using anti-CD3 and anti-CD28.

FIG. 12

Graph demonstrating that anti-TIGIT H03 antibody reversed CD155-mediated CD8+ T cell suppression by increasing IFNγ production in a CD8+ T cell activation assay using anti-CD3.

FIG. 13

Graphs demonstrating the binding of H03-12 to human (A) and cynomolgus monkey (B) CD3+ T cells.

FIG. 14

Graphs demonstrating dose dependent target occupancy of H03-12 in human whole blood (A) and cynomolgus monkey spleen cells (B).

FIG. 15

Graphs demonstrating that H03-12 dose-dependently blocked TIGIT/CD155 (A) and TIGIT/CD112 (B) interaction.

FIG. 16

Graphs demonstrating the setup of a FRET-based TIGIT/CD226 blocking assay (A) and dose-dependent inhibition of TIGIT/CD226 interaction by 3963H03-12 (B).

FIG. 17

Graph demonstrating dose-dependent activity of 3963H03-12 in the two-way MLR assay.

FIG. 18

Graph demonstrating dose-dependent activity of 3963H03-12 in the one-way MLR allo assay.

FIG. 19

Graphs demonstrating that 3963H03-12 enhanced NK cell activation in NK cell-mediated killing assays using P815.hCD155 cells (A) and MDA-MB-231 GFP/Luc cells (B).

FIG. 20

Graph demonstrating blocking potency of 3963H03-12 and 3963H03-12-muIgG2c on the binding of muCD155 and muCD112 to CHO-S-huTIGIT cells.

FIG. 21

Pharmacokinetic evaluation of 3963H03-12-muIgG2c in B-huTIGIT knock-in mice bearing MC38 tumors

FIG. 22

Graphs demonstrating anti-tumor efficacy of 3963H03-12-muIgG2c in MC38 colon carcinoma model (A), GL261 glioblastoma model (B), Hepa 1-6 hepatocellular carcinoma model (C) and 3LL lung carcinoma model (D) in B-huTIGIT knock-in mice

FIG. 23

Graphs showing the dose-dependent anti-tumor efficacy of 3963H03-12-muIgG2c in the MC38 tumor model in B-huTIGIT knock-in mice. Average and individual tumor volumes are plotted for each treatment group in addition to median survival in days.

FIG. 24

Graphs demonstrating that effector competent 3963H03-12-muIgG2c, but not effector null 3963H03-12-muIgG1(D265A), had anti-tumor efficacy in the MC38 model (A) or Hepa 1-6 model (B) in B-huTIGIT knock-in mice

FIG. 25

Graphs showing results of combination treatment with 3963H03-12-muIgG2c and avelumab in MC38 tumor model in B-huTIGIT knock-in mouse comparing both average and individual tumor volumes for each treatment group in addition to median survival in days.

FIG. 26

Results of combination treatment with 3963H03-12-muIgG2c and bintrafusp alfa in MC38 tumor model in B-huTIGIT knock-in mouse. Both averaged and individual tumor volumes demonstrate enhanced anti-tumor efficacy with the combination treatment compared to either monotherapy. Prolonged survival is also observed with the combination relative to either monotherapy.

FIG. 27

Results of re-challenge studies performed on MC38 tumor-bearing B-huTIGIT knock-in mice that showed complete tumor regression after combination treatment with 3963H03-12-muIgG2c and either avelumab or bintrafusp alfa. Tumor volumes are shown of naïve mice compared to cured mice.

### EXPERIMENTAL SECTION

The working examples presented below are intended to illustrate particular embodiments of the invention and are not intended to limit the scope of the specification or the claims in any way.

1. Selection and Improvement of Antibodies

To generate fully human monoclonal antibodies to TIGIT, OmniRats (Open Monoclonal Technologies, Inc./Ligand Pharmaceutical Inc.) were immunized with the recombinant extracellular domain (ECD) of human TIGIT (Sino Biological Inc, Cat. 10917-H08H) using a Repetitive Immunization at Multiple Sites strategy (also known as RIMMS). Eight to twelve week old rats were immunized biweekly for six times with recombinant TIGIT protein emulsified with complete Freund's Adjuvant (Sigma-Aldrich, Cat. F5881) for the first injection followed by incomplete Freund's Adjuvant (Sigma-Aldrich, Cat. F5506) for the remaining injections. The serum immune response was monitored by ELISA against the immunogen. Briefly, a 96-well clear flat bottom plate (Thermo Scientific, Cat. 439454) was coated with human TIGIT protein (Sino Biological Inc, Cat. 10917-H08H) at 4° C. overnight. Plates were washed with PBS/0.05% Tween 20 and incubated with 3% BSA (Sigma, Cat. A3912-100G) for 2 hours at room temperature. Serially diluted serum samples were added into the plates and incubated 1 hour at room temperature. The plates were then incubated with 1:5000 diluted horse radish peroxidase-conjugated goat anti-rat IgG Fc fragment (Jackson ImmunoResearch, Cat. 112-036-071) for 1 hour. The color was developed with 100 ul of tetramethyl benzidine hydrochloride (TMB) substrate (BioFx, Cat. TMBW-1000-01) and stopped with the addition of 50 ul of 2N sulfuric acid (Sigma Aldrich, Cat. 320501-500). The absorbance at 450 nm was read using a SpectraMax M5 (Molecular Devices).

Single B cell sorting was performed from the lymphocytes collected from blood and/or spleen and/or lymph nodes from immunized rats with high serum immune response. In short, cells were incubated with anti-rat CD32 (clone D34-485, BD Biosciences) for 5 minutes followed by human TIGIT protein (R&D, cat. #7898-TG) for 1 hour at 4° C. Cells were then washed and incubated with a mixture of FITC-conjugated mouse anti-rat IgM (clone MRM-47, Biolegend), PE-Cy7-conjugated mouse anti-rat CD45R (clone HIS 24, eBioscience), and APC-conjugated mouse anti-His (clone AD1.1.10R, R&D) antibodies for 30 minutes at 4° C. Single TIGIT+ B cells were sorted into each well of a 96 well plate containing 4 ul lysis buffer (0.1M DTT, 40 U/ml Rnase Inhibitor, Invitrogen, Cat #10777-019) on BD FACS Aria III flow cytometer. Plates were sealed with Microseal ‘F’ Film (BioRad) and immediately frozen on dry ice before storage at −80° C.

Ig V-gene cloning from single sorted B cell was performed with a protocol modified from Tiller et al., 2008, J Imm Methods 329. In brief, total RNA from single sorted B cells was reverse transcribed in a final volume of 14 μl/well in the original 96-well sorting plate with nuclease-free water (Invitrogen, Cat #AM9935) using final amounts/concentrations of 150 ng random hexamer primer (pd(N)6, Applied Biosystems, P/N N808-0127) and 50U Superscript III reverse transcriptase (Invitrogen, Cat #18080-044) following the manufacturer's protocol. Primers (not listed) were modified based on previous publications (Wardemann et al, Science 2003 301:1374-1377) and/or designed by examining published Ig gene segment nucleotide sequences from IMGT®, the international ImMunoGeneTics information system (http://www.imgt.org; (Lefranc et al., 2009) and NCBI (http://www.ncbi.nlm.nih.gov/igblast/) databases. Human Igh, Igk and Igl V gene transcripts were amplified independently by two rounds of nested (Igh, Igk and Igl) PCR starting from 3.5 μl of cDNA as template. All PCR reactions were performed in 96-well plates in a total volume of 40 μl per well using AccuPrime Taq DNA Polymerase High Fidelity kit, (Invitrogen, Cat #. 12346-094) following the manufacturer's protocol. The first round of PCR was performed at 95° C. for 2 min followed by 40 cycles of 94° C. for 30 s, 50° C. for 30 s, 72° C. for 40 s, and final incubation at 72° C. for 5 min.

Nested second round PCR was performed with 5 μl of unpurified first round PCR product at 95° C. for 2 min followed by 5 cycles of 94° C. for 30 s, 42° C. for 30 s, 72° C. for 45 s, and then 50 cycles of 94° C. for 30 s, 55° C. for 30 s, 72° C. for 45 s, and final incubation at 72° C. for 5 min. PCR products were cloned into IgG expression vectors for Ig expression and functional screening.

A total of 860 TIGIT+ B cells were isolated by single cell fluorescence-activated cell sorting. Immunoglobulin VH and VL regions were PCR amplified from cDNA prepared from the individual lysed B cells. Paired VH and VL regions were obtained from 388 B cell lysates and cloned into IgG expression vectors for expression, and biochemical characterization, and DNA sequencing.

Hit optimization candidates were selected based on the potency to block binding of CD155 to TIGIT and the ability to bind to both human TIGIT and cynomolgus monkey TIGIT. Binding to TIGIT was originally determined by ELISA and to TIGIT expressing cells by FACS, and later quantified by Biacore. Eighty-three clones were confirmed by ELISA and flow cytometry assays as human and cynomolgus monkey TIGIT (Novoprotein cat. No. CP02) cross-reactive cell binders. Thirty of these clones blocked the TIGIT:CD155 interaction. Four candidates, 3963H03, 3964A06, 3965D08, and 3966C11 (also abbreviated as H03, A06, D08, and C11, respectively) fitted the predefined profile and ultimately 3963H03 was chosen for sequence optimization. The goals of the sequence optimization were to replace non-germline residues in the variable region frameworks with germline residues and to improve the manufacturability by removing sequence motifs potentially prone to post-translational modification.

The heavy and light chain amino acid sequences of 3963H03 are as follows:

Heavy chain:
(SEQ ID NO: 22)
EIQLVQSGSELKKPGASVKVSCKASGYTFTSYPMN
WRQAPGQGLEWMGWINTNTGNPTYAQGFTGRFVFS
LDTSVSTAYLQISSLKAEDTAVYYCARVGGYSVDE
YAFDVWGQGTMVTVSSASTKGPSVFPLAPSSKSTS
GGTAALGCLVKDYFPEPVTVSWNSGALTSGVHTFP
AVLQSSGLYSLSSVVTVPSSSLGTQTYICNVNHKP
SNTKVDKRVEPKSCDKTHTCPPCPAPELLGGPSVF
LFPPKPKDTLMISRTPEVTCVVVDVSHEDPEVKFN
WYVDGVEVHNAKTKPREEQYNSTYRVVSVLTVLHQ
DWLNGKEYKCKVSNKALPAPIEKTISKAKGQPREP
QVYTLPPSREEMTKNQVSLTCLVKGFYPSDIAVEW
ESNGQPENNYKTTPPVLDSDGSFFLYSKLTVDKSR
WQQGNVFSCSVMHEALHNHYTQKSLSLSPG
Light chain:
(SEQ ID NO: 23)
AIRLTQSPSFLSASVGDRVTITCRASQGISSYLAW
YQQKPGKAPKLLIYAASTLQSGVPSRFSGSGSGTE
FTLTISSLQPEDFATYYCQQLNSYPTFGGGTKVEI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC

Yeast Display AFM and Sequence Optimization

3963H03 heavy and light chain CDR3 regions were subjected to parsimonious mutagenesis and used to construct yeast display libraries for obtaining affinity matured variants. Two libraries were constructed initially: (1) a mutagenized L3-CDR light chain library paired with the parental heavy chain, and (2) a mutagenized H3-CDR heavy chain library paired with the parental light chain. The L3-CDR and H3-CDR libraries were screened separately by FACS for 2 rounds to select for the top 5-10% binders having a higher signal than a yeast clone expressing parental 3963H03. The mutagenized light chains and heavy chains were isolated from pools resulting from the selections and transformed into yeast to make new libraries having reduced complexities. From these, mating libraries were made and screened for up to three rounds to isolate the top 1-0.1% high affinity binders by FACS. Selected and validated clones with higher affinities were subcloned into a mammalian expression vector for further validation by BIACORE™ affinity analysis. Two candidates, 7729G05 and 7728B03, demonstrated affinities for human and cynomolgous monkey TIGIT in the picomolar KD range, and were selected for further studies.

An assessment of the variable region sequences of 3963H03 identified two non-germline amino acid residues in the light chain variable region framework and two non-germline amino acids in the heavy chain variable region framework. Additionally, a methionine residue within the heavy chain framework 4 was identified which could potentially oxidize overtime, and a deamidation motif was identified in the light chain CDR3. Based on these analyses, a series of sequence designs were generated in which the potentially problematic amino acids were replaced with either the germline-associated amino acid at that position or, in the case of the germline methionine, with a biophysically conservative leucine substitution. The amino acid substitutions are shown in Table 1

TABLE 1
Amino acid substitution variants of 3963H03 heavy and light chain V regions.

| Heavy chain positions | | | Light chain positions | | | | |
|---|---|---|---|---|---|---|---|
| IMGT numbering | 1, 2 | 123 | IMGT numbering | 1 | 3 | 108 | 114 |
| Kabat numbering | H1, H2 | H108 | Kabat numbering | LI | L3 | 92 | 93 |
| Sequential numbering | 1, 2 | 117 | Sequential numbering | 1 | 3 | 92 | 93 |
| Region | FR1 | FR4 | Region | FR1 | FR1 | CDR3 | CDR3 |
| Parent ID | EI | M | Parent ID | A | R | N | S |
| Germline ID | QV | L | Germline ID | D | Q | N | S |
| Substitution Designs | QV | L | Substitution Designs | D | Q | NSQ | ASQ |
| VH1.00 (parental) | EI | M | VL1.00 (parental) | A | R | N | S |
| VH1.01 | QV | M | VL1.01 | D | Q | N | S |
| VH1.02 | EI | L | VL1.02 | D | Q | S | S |
| VH1.03 | QV | L | VL1.03 | D | Q | Q | S |
| | | | VL1.04 | D | Q | N | A |
| | | | VL1.05 | D | Q | N | Q |

The sequence optimized variant designated H03-12, consisting of VH1.03 (E1Q, I2V, M117L, sequential numbering) and VL1.02 (A1D, R3Q, N92S, sequential numbering), was selected as the lead candidate based on having the most favorable substitutions, good productivity in CHO cells, and activity equivalent to, or better than, the parental molecule in the binding and functional assays.

1.1 Variant Identification by NGS and SPR

3963H03-related B cell sequences were expanded through Next Generation Sequencing (NGS) technology. Briefly, from the same lymph node tissue used to clone 3963H03, about 5×10⁵ TIGIT-specific B cells and plasma cells were collected using FACS through bulk sorting. Total RNA was isolated to generate the NGS library. Following cDNA synthesis, IGVH7-4 and IGKV1-9 (relevant to the 3963H03 hit) gene-specific primers were used for RT-PCR isolation of the 3963H03 specific IgH and IgK B cell V-region sequences; these were subcloned into expression vectors for IgG antibodies. 73 VH sequences related to 3963H03 with unique CDR sequences (Table 2) were paired with the parental light chain of 3963H03 and expressed as IgG in Expi293F cells. Similarly, 20 VK sequences related to 3963H03 with unique CDR sequences (Table 3) were paired with the parental heavy chain of 3963H03 and expressed as IgG in Expi293F cells. Culture supernatants for these 93 IgGs, as well as 39603H03 parental, were collected, diluted 1:10, and the kinetic off-rates of binding to human TIGIT were measured by Surface Plasmon Resonance (SPR) using a GE Healthcare Biacore 4000 instrument as follows. Goat anti-human Fc antibody (Jackson Immunoresearch Laboratories #109-005-098) was first immobilized on a BIAcore carboxymethylated dextran CM5 chip using direct coupling to free amino groups following the procedure described by the manufacturer. Antibodies were then captured on the CM5 biosensor chip. Binding measurements were performed using the running HBS-EP+ buffer. Two-fold dilution series of his-tagged human TIGIT, with starting concentrations between 100 nM and 10 nM, were injected at a flow rate of 30 μl/min at 25° C. Dissociation rates (koff, s-1) were calculated using a simple 1:1 Langmuir binding model (Biacore Evaluation Software). The measured koff are shown in Table 2 and Table 3, revealed both slower and faster koff among the new variants.

1.2 High Affinity Variant Identification by Pairwise Additivity of Heavy Chains and Light Chains.

We hypothesized that k_{off} gains or losses would be pair-wise additive, as illustrated mathematically in Equation 1.

$${k}_{off ( {VH}_{i} , {VL}_{j} )} = \frac{{k}_{off ( {VH}_{i} , {VL}_{nat} )}}{{k}_{off ( {VH}_{nat} , {VL}_{nat} )}} \times \frac{{k}_{off ( {VH}_{nat} , {VL}_{j} )}}{{k}_{off ( {VH}_{nat} , {VL}_{nat} )}} . \qquad Equation 1$$

Using Equation 1, the off-rates of the antibodies in Tables 2 and 3 were used to predict the activity of variant heavy or light chains paired with other variant light or heavy chains. 15 NGS identified variant pairs were predicted to have improved binding affinity. These 15 were expressed and the kinetic off-rate characterized by SPR. Three of these were within 1.5 fold of 3963H03 while 12 variants had improved affinity of between 2 and 4.7 fold relative to 3963H03 as predicted (Table 4). Overall, this strategy together with the previous one allowed for identification of variants with a range of activities, including many with improved off-rate relative to the initial hit 3963H03, of which the sequence was used as a probe to generate the library.

TABLE 2
73 VH sequences with unique CDRs related
to 3963H03 were identified by NGS.
These were paired with the light chain
of 3963H03 and the koff was measured.

| | | | | koff |
|---|---|---|---|---|
| | | | | (second |
| ID | HVR-H1 | HVR-H2 | HVR-H3 | 1) |
| 3963H03 | GYTFTSYP | INTNTGNP | ARVGGYSVDEYAFDV | 6.3E−04 |
| VH | | | | |
| D04H01 | GYTFTSYA | INTNTGNP | ARVGGYSGYDYAFDI | 8.3E−03 |
| D04H02 | GYTFTSYA | INTNTGNP | ARVGGYSGYDYAFDV | 7.0E−03 |
| D04H09 | GYTFTSYP | INTNTGNP | ARVGGYSGYDYAFDI | 1.4E−03 |
| D04H10 | GYTFTSYP | INTNTGNP | ARVGGYSGYDYAFDV | 1.4E−03 |
| D04H11 | GYTFTSYP | INTNTGNP | ARVGGYGGYDYAFDI | 5.1E−03 |
| D04H12 | GYTFTSYP | INTNTGNP | ARVGGYGVYDYAFDV | 4.3E−03 |
| D04H13 | GYTFTSYP | INTNTGNP | ARVGGYIVYDYAFDV | 3.7E−04 |
| D04H15 | GYTFTSYP | INTNTGNP | ARVGGYGGYDYAFDV | 6.1E−03 |
| D04H16 | GYTFTSYP | INTNTENP | ARVGGYGGYDYAFDI | 6.6E−03 |
| D04H17 | GYTFTAYA | INTNTGNP | ARVGGYGGYDYAFDI | 4.2E−03 |
| D04H18 | GYTFTAYP | INTNTGNP | ARVGGYSGYDYAFDI | 1.2E−03 |
| D04H19 | GYTFTAYP | INTNTGNP | ARVGGYSVNDYAFDI | 4.8E−04 |
| D04H20 | GYTFTAYP | INTNTGNP | ARIGGYSVNDYAFDI | 1.6E−03 |
| D04H23 | GYTFTTYP | INTNTGNP | ARVGGYSGYDYAFDI | 2.6E−03 |
| D04H24 | GYTFTTYP | INTNTGNP | ARVGGYGVYDYAFDV | 7.9E−03 |
| D04H25 | GYTFTTYP | INTNTENP | ARVGGYGGYDYAFDV | 2.8E−02 |
| D04H26 | GYTFASYP | INTNTGNP | ARVGGYGGYDYAFDI | 6.0E−02 |
| D04H27 | GYTLTSYP | INTNTGNP | ARVGGYGGYDYAFDF | 4.9E−03 |
| D04H28 | GYTLTSYP | INTNTGNP | ARVGGYGGHDYAFDI | 6.4E−03 |
| D04H29 | GYIVTSYA | INTNTGNP | ARVGGYSGYDYAFDI | 3.2E−03 |
| D04H36 | GYTFTAYA | INTNTGNP | ARVGGYGVYDYAFDI | 9.7E−03 |
| D04M07 | GYTFTSYA | INTNTGNP | ARVGGYSGFDYAFDI | 6.4E−03 |
| D04M11 | GYTFTSYP | INTNTGNP | ARVGGYSGYDYGFDI | 2.8E−03 |
| D04M12 | GYTFTSYP | INTNTGNP | ARVGGYSGNDYAFDI | 2.3E−03 |
| D04M21 | GYTFTSYP | INTNTGDP | ARVGGYSGYDYAFDI | 1.0E−03 |
| D04M22 | GYTFTSYP | INTNTANP | ARVGGYSGYDYAFDI | 1.6E−03 |
| D04M27 | GYTFTSFP | INTNTGNP | ARVGGYSGYDYAFDI | 2.2E−03 |
| D04M28 | GYTFTSFP | INTNTGNP | ARVGGYSGYDYAFDM | 2.5E−03 |
| D04M29 | GYTFTSFP | INTNTGNP | ARVGGYGGYDYAFDI | 4.9E−03 |
| D04M30 | GYTFTSFP | INTNTGNP | ARVGGYGGSDYAFDI | 9.1E−03 |
| D04M32 | GYTFTNYA | INTNTGNP | ARVGGYSGYDYAFDI | 4.9E−03 |
| D04M33 | GYTFTNYP | INTNTGNP | ARVGGYSGYDYAFDI | 1.9E−03 |
| D04M35 | GYTFTYYP | INTNTGNP | ARVGGYSGYDYAFDI | 1.1E−03 |
| D04M36 | GYTFASYP | INTNTGNP | ARVGGYSGYDYAFDI | 1.3E−03 |
| D04M37 | GYTFISYP | INTNTGNP | ARVGGYSGYDYAFDI | 1.3E−03 |
| D04M39 | GYTFPSYP | INTNTGNP | ARVGGYSGSDYAFDI | 2.3E−03 |
| D04M41 | GYTFSSYP | INTNTGNP | ARVGGYSGYDYAFDI | 1.4E−03 |
| D04M42 | GYTFSSYP | INTNTGNP | ARVGGYGGYDYAFDI | 3.8E−03 |
| D04M44 | GYTFSSYP | INTNTGNP | ARVGGYGVYDYAFDI | 4.3E−03 |
| D04M47 | GYTFSSYP | INTNTGNP | ARVGGYGGDDYAFDI | 2.8E−02 |
| D04M49 | GYAFTSYP | INTNTGNP | ARVGGYSGYDYAFDI | 1.2E−03 |
| D04M51 | GYAFTTYP | LNTNTGNP | ARVGGYGGYDYAFDI | 3.6E−03 |
| D04M52 | GYAFSTYA | INTNTGNP | ARVGGYGGYDYAFDI | 3.6E−03 |
| D04M53 | GYAFSTYA | LNTNTGNP | ARVGGYGGYDYAFDI | 4.3E−03 |
| D04M54 | GYIFTSYP | INTNTGNP | ARVGGYSGYDYAFDI | 1.3E−03 |
| D04M55 | GYSFTSYP | INTNTGNP | ARVGGYGGYDYAFDI | 3.1E−03 |
| D04M56 | GYSFTDYP | INTNTGNP | ARVGGYSGYDYAFDI | 1.8E−03 |
| D04M58 | GDTFRSYP | INTNTGNP | ARVGGYGGYDYAFDI | 6.0E−03 |
| D04M59 | GFTFTSYP | INTNTGNP | ARVGGYSGYDYAFDF | 4.5E−03 |
| D04M60 | GNTFTSYP | INTNTGNP | ARVGGYSGYDYAFDI | 4.3E−03 |
| D04M61 | GYTFTNYP | INTNTGNP | ARVGGYSGYDYSFDI | 2.0E−03 |
| D04M64 | GYSFTSYP | INTNTGNP | ARVGGYSGYDYAFDI | 1.1E−03 |
| D04M65 | GYSFTNYP | INTNTGNP | ARVGGYSGYDYAFDI | 1.3E−03 |
| D04M66 | GYTFTTYP | INTNTGNP | ARVGGYSAYDYAFDI | 1.7E−03 |
| H03H01 | GYTFTSYA | INTNTGNP | ARVGGYSVYDYAFDI | 3.5E−03 |
| H03H02 | GYTFTSYP | INTNTGNP | ARVGGYSVYDYAFDI | 6.2E−04 |
| H03H03 | GYTFTSYP | INTNTGNP | ARVGGYSVYDYAFDA | 7.5E−04 |
| H03H04 | GYTFTSYP | INTNTGNP | ARVGGYSVYDYAFDV | 6.0E−04 |
| H03H06 | GYTFTSYP | INTNTGNP | ARVGGYSVYDYASDV | 4.2E−03 |
| H03H08 | GYTFTSYP | INTNTGNP | ARVGGYSVDDYAFDV | 5.7E−04 |
| H03H15 | GYTFTAYA | INTNTGNP | ARVGGYSVYDYAFDI | 2.8E−03 |
| H03H16 | GYTFTAYP | INTNTGNP | ARVGGYSVYDYAFDI | 6.4E−04 |
| H03H17 | GYTFTAYP | INTNTGNP | ARVGGYSVYDYAFDV | 5.5E−04 |
| H03H18 | GYTFTAYP | INTNTGNS | ARVGGYSVYDYAFDI | 8.6E−04 |
| H03H19 | GYTFTAYP | INTNTGSP | ARVGGYSVYDYAFDI | 5.0E−04 |
| H03H22 | GYTFTTYA | INTNTGNP | ARVGGYSVYDYAFDI | 5.7E−03 |
| H03H23 | GYTFTTYP | INTNTGNP | ARVGGYSVYDYAFDI | 1.1E−03 |
| H03H24 | GYTFTTYP | INTNTGNP | ARVGGYSVYDYAFDV | 1.0E−03 |
| H03M02 | GYAFSAYA | LNTNTGNP | ARVGGYSVYDYAFDI | 2.5E−02 |
| H03M03 | GYSFTNYP | INTNTGNP | ARVGGFSDYDYAFDI | 1.9E−03 |
| H03M04 | GSTFTSYP | INTNTGNP | ARVGGYSAYDYAFDI | 5.5E−03 |
| H03M05 | GYTFTNYA | INTNTGNP | ARVGGYSDYDYAFDI | 1.4E−02 |
| H03M07 | GYTFTSYP | INTNTGNP | ARVGGYSDYDYAFDI | 1.4E−03 |

TABLE 3
20 VK sequences with unique CDRs related
to 3963H03 were identified by NGS.
These were paired with the heavy chain
of 3963H03 and the koff was measured..

| ID | HVR-H1 | HVR-H2 | HVR-H3 | koff (second 1) |
|---|---|---|---|---|
| 3963H03 VK | QGISSY | AAS | QQLNSYPT | 6.3E−04 |
| H03K02 | QGISSY | AAS | QQLNSYLT | 1.7E−03 |
| H03K03 | QGISSY | AAS | QQLNGYLT | 2.5E−03 |
| H03K04 | QAISSY | AAS | QQLNGYLT | 1.6E−03 |
| H03K05 | QGISSY | AAS | QQLNNYLT | 2.0E−03 |
| H03K06 | QGISSY | GAS | QQLNGYPT | 2.7E−04 |
| H03K07 | QGISSY | GAS | QQLNSYPT | 3.4E−04 |
| H03K08 | QVISSY | AAS | QQLNSYPT | 3.6E−04 |
| H03K09 | QGISSY | AAS | QQLNSYPL | 5.6E−04 |
| H03K10 | QGISSY | AAS | QQLNSYPP | 8.8E−04 |
| H03K11 | QGISSY | AAS | QQLNGYPT | 2.8E−04 |
| H03K12 | QGISSY | AAS | QQLNGSPT | 3.5E−04 |
| H03K15 | QGISSS | AAS | QQLNSYPT | 3.4E−04 |
| H03K24 | QGIPSY | AAS | QQLNSYPT | 3.6E−04 |
| H03K25 | QAISSY | AAS | QQLNSYPT | 4.6E−04 |
| H03K26 | QGISSY | AAS | QQPNGYLT | 4.2E−04 |
| H03K29 | QGISTY | AAS | QQLNSYLT | 1.6E−03 |
| H03K30 | QGINSY | AAS | QQLNSYPT | 3.4E−04 |
| H03K31 | QAISSY | AAS | QQPNGYLT | 6.5E−04 |
| H03K34 | QGISSY | AAS | QQLNSYPH | 6.1E−04 |
| H03K35 | QGISSY | AAS | QQLNSYIT | 6.2E−04 |

TABLE 4
Novel pairings of VH & VK sequences related to 3963H03 identified by
NGS and predicted to have a slower koff

| ID of heavy | ID of light | koff (second⁻¹) of |
|---|---|---|
| from Table 2 | from Table 3 | novel variant |
| D04H13 | H03K06 | 1.5E−04 |
| D04H13 | H03K08 | 1.3E−04 |
| D04H13 | H03K11 | 1.7E−04 |
| D04H13 | H03K15 | 2.1E−04 |
| D04H13 | H03K30 | 1.6E−04 |
| H03H08 | H03K06 | 1.4E−04 |
| H03H08 | H03K11 | 2.7E−04 |
| H03H08 | H03K12 | 2.7E−04 |
| H03H08 | H03K15 | 2.3E−04 |
| H03H08 | H03K19 | 3.1E−04 |
| H03H08 | H03K21 | 3.1E−04 |
| H03H08 | H03K26 | 6.3E−04 |
| H03H08 | H03K30 | 5.3E−04 |
| H03H12 | H03K06 | 2.1E−04 |
| H03H24 | H03K06 | 4.6E−04 |

2. Manufacturing and Purification

2.1 Bioproduction, Clarification and Purification

The antibody H03-12 as disclosed was produced from CHO-K1SV cells.

3963H03-12 heavy chain:
(SEQ ID NO: 18)
QVQLVQSGSELKKPGASVKVSCKASGYTFTSYPMN
WWRQAPGQGLEWMGWINTNTGNPTYAQGFTGRFVF
SLDTSVSTAYLQISSLKAEDTAVYYCARVGGYSVD
EYAFDVWGQGTLVTVSSASTKGPSVFPLAPSSKST
SGGTAALGCLVKDYFPEPVTVSWNSGALTSGVHTF
PAVLQSSGLYSLSSVVTVPSSSLGTQTYICNVNHK
PSNTKVDKRVEPKSCDKTHTCPPCPAPELLGGPSV
FLFPPKPKDTLMISRTPEVTCVVVDVSHEDPEVKF
NWYVDGVEVHNAKTKPREEQYNSTYRVVSVLTVLH
QDWLNGKEYKCKVSNKALPAPIEKTISKAKGQPRE
PQVYTLPPSREEMTKNQVSLTCLVKGFYPSDIAVE
WESNGQPENNYKTTPPVLDSDGSFFLYSKLTVDKS
RWQQGNVFSCSVMHEALHNHYTQKSLSLSPG.
3963H03-12 light chain:
(SEQ ID NO: 19)
DIQLTQSPSFLSASVGDRVTITCRASQGISSYLAW
YQQKPGKAPKLLIYAASTLQSGVPSRFSGSGSGTE
FTLTISSLQPEDFATYYCQQLSSYPTFGGGTKVEI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC.

Cells were grown in proprietary-CHO fed-batch growth media supplemented with glucose at 37° C. The cultures were fed with a mixture of proprietary feed components on days 3, 5, 7 and 10 days post inoculation.

Crude conditioned media from the bioreactor runs were clarified using 2.2 m2 Millistak+ Pod D0HC (Millipore MD0HC10FS1) and 1.1 m2 Millistak+ Pod X0HC (Millipore #MX0HC01FS1) filters, followed by terminal filtration with a Millipore Opticap XL3 0.5/0.2 μm filter (Millipore #KHGES03HH3).

The antibody was then purified using standard methods and formulated in 10 mM Histidine, 5 mM Methionine, 8% Trehalose, pH 5.5 with 0.05% Tween 20.

The antibody can be stored in phosphate buffer, pH adjusted with NaCl as isotoning agent.

3. Biochemical and Biological Characterization

3.1 Biacore Binding Affinity and Specificity

Binding affinities of anti-TIGIT hit candidate antibodies to human TIGIT and cynomolgus monkey TIGIT were measured by Surface Plasmon Resonance (SPR) using a GE Healthcare Biacore 4000 instrument and a GE Healthcare Biacore T200 instrument as follows. Goat anti-human Fc antibody (Jackson Immunoresearch Laboratories #109-005-098) was first immobilized on BIAcore carboxymethylated dextran CM5 chip using direct coupling to free amino groups following the procedure described by the manufacturer. Antibodies were then captured on the CM5 biosensor chip to achieve approximately 200 response units (RU). Binding measurements were performed using the running HBS-EP+ buffer. 2-fold dilution series with starting concentration between 100 nM and 10 nM of His-tagged human and cynomolgus monkey (cyno) TIGIT proteins were injected at a flow rate of 30 μl/min at 25° C. Association rates (kon, M-1 s-1) and dissociation rates (koff, s-1) were calculated using a simple 1:1 Langmuir binding model (Biacore Evaluation Software). The equilibrium dissociation constant (KD, M) was calculated as the ratio of koff/kon. Affinity of candidates 3963H03, 3963H03-12, 3964A06, 3965D08, and 3966C11 for binding to human TIGIT ranged from 2.5 to 10 nM and affinity for binding to cyno TIGIT ranged from 0.8 to 8.7 nM (Table 5).

TABLE 5
Biacore affinity measurements for anti-TIGIT hit candidates from OmniRat

| Antibody | Human TIGIT | | | | Cyno TIGIT | | | |
|---|---|---|---|---|---|---|---|---|
| ID | ka(M-1 sec-1) | kd(sec-1) | KD(M) | KD(nM) | ka(M-1 sec-1) | kd(sec-1) | KD(M) | KD(nM) |
| 3963H03 | 1.21E+05 | 4.07E−04 | 3.37E−09 | 3.4 | 1.47E+05 | 2.13E−04 | 1.45E−09 | 1.5 |
| 3964A06 | 8.91E+04 | 2.33E−04 | 2.62E−09 | 2.6 | 1.09E+05 | 1.22E−04 | 1.11E−09 | 1.1 |
| 3965D08 | 4.82E+05 | 4.97E−03 | 1.03E−08 | 10.3 | 6.53E+05 | 5.65E−03 | 8.66E−09 | 8.7 |
| 3966C11 | 1.08E+05 | 2.74E−04 | 2.53E−09 | 2.5 | 1.18E+05 | 1.67E−04 | 1.41E−09 | 1.4 |
| 3963H03-12 | 1.66E+05 | 6.70E−04 | 4.05E−09 | 4.05 | 1.60E+05 | 1.33E−04 | 8.29E−10 | 0.83 |
| 7728B03 | 3.25E+05 | 2.15E−05 | 6.61E−11 | 0.07 | 3.03E+05 | 1.57E−05 | 5.19E−11 | 0.05 |
| 7729G05 | 7.67E+05 | 1.33E−05 | 1.73E−11 | <0.051 | 5.81E+05 | 1.23E−05 | 2.11E−11 | <0.05¹ |

¹Affinity measurement is below the sensitivity of the Biacore instrument (50 pM)

Table 6a shows the CDR sequences of the candidate antibodies described herein.

TABLE 6a
Anti-TIGIT CDR's of different anti TIGIT candidates

| | HVR-H1 | HVR-H2 | HVR-H3 | HVR-L1 | HVR-L2 | HVR-L3 |
|---|---|---|---|---|---|---|
| H03 | GYTFTSYP | INTNTGNP | ARVGGYSVDEYAFDV | QGISSY | AAS | QQLNSYPT |
| H03-12 | GYTFTSYP | INTNTGNP | ARVGGYSVDEYAFDV | QGISSY | AAS | QQLSSYPT |
| A06 | GYTFTAYP | INTNTGNP | ARVGGYSVYDYAFDI | QGISSY | AAS | QQLNSYPT |
| D08 | GYTFTSYP | INTNTGNP | ARTGYSGSYYWFDP- | QGISSY | AAS | QQLNSYLT |
| C11 | GYTFTSYP | INTNTGNP | ARVGGYGGYDYAFDI | QGISSY | AAS | QQLNSYPT |
| B03 | GYTFTSYP | INTNTGNP | ARTGGYSVDEYSFDI | QGISSY | AAS | HQTIFRPT |
| G05 | GYTFTSYP | INTNTGNP | ARVGGFTVPEYAFDI | QGISSY | AAS* | GQVMRYPA |

Table 6b shows deviations in the framework region sequences as compared to antibody

TABLE 6B
Anti-TIGIT FR’s of different anti TIGIT candidates,
as compared to H03-12

| | VL region residue position | | | | | | VH region residue position | | | |
|---|---|---|---|---|---|---|---|---|---|---|
| | FR1 | | FR2 | FR4 | | | FR1 | | FR3 | FR4 |
| | 1 | 3 | 46 | 99 | 102 | 103 | 1 | 2 | 60 | 117 |
| H03 | A | R | L | G | K | V | E | I | Y | M |
| A06 | D | Q | L | G | K | V | E | V | Y | M |
| D08 | D | R | F | Q | R | L | E | V | Y | L |
| C11 | D | Q | L | G | K | L | E | I | N | M |
| H03-12 | D | Q | L | G | K | V | Q | V | Y | L |
| B03 | D | Q | L | G | K | V | Q | M | Y | T |
| G05 | D | R | L | G | K | V | Q | V | Y | T |

framework region amino acids as well as the constant regions corresponds to those of H03-12.

The full variable region sequences are provided in the following:

A06-VH
(SEQ ID NO: 24)
EVQLVQSGSELKKPGASVKVSCKASGYTFTAYPMNWVRQAPGQGLEWMGW
INTNTGNPTYAQGFTGRFVFSLDTSVSTAYLQISSLKAEDTAVYYCARVG
GYSVYDYAFDIWGQGTMVTVSS
A06-VL
(SEQ ID NO: 25)
DIQLTQSPSFLSASVGDRVTITCRASQGISSYLAWYQQKPGKAPKLLIYA
ASTLQSGVPSRFSGSGSGTEFTLTISSLQPEDFATYYCQQLNSYPTFGGG
TKVEIK
C11-VH
(SEQ IN NO: 26)
EIQLVQSGSELKKPGASVKVSCKASGYTFTSYPMNWVRQAPGQGLEWMGW
INTNTGNPTNAQGFTGRFVFSLDTSVSTAYLQISSLKAEDTAVYYCARVG
GYGGYDYAFDIWGQGTMVTVSS
C11-VL
(SEQ IN NO: 27)
DIQLTQSPSFLSASVGDRVTITCRASQGISSYLAWYQQKPGKAPKLLIYA
ASTLQSGVPSRFSGSGSGTEFTLTISSLQPEDFATYYCQQLNSYPTFGGG
TKLEIK
H03-VH
(SEQ ID NO: 28)
EIQLVQSGSELKKPGASVKVSCKASGYTFTSYPMNWVRQAPGQGLEWMGW
INTNTGNPTYAQGFTGRFVFSLDTSVSTAYLQISSLKAEDTAVYYCARVG
GYSVDEYAFDVWGQGTMVTVSS
H03-VL
(SEQ ID NO: 29)
AIRLTQSPSFLSASVGDRVTITCRASQGISSYLAWYQQKPGKAPKLLIYA
ASTLQSGVPSRFSGSGSGTEFTLTISSLQPEDFATYYCQQLNSYPTFGGG
TKVEIK
D08-VH
(SEQ ID NO: 30)
EVQLVQSGSELKKPGASVKVSCKASGYTFTSYPMNWVRQAPGQGLEWMGW
INTNTGNPTYAQGFTGRFVFSLDTSVSTAYLQISSLKAEDTAVYYCARTG
YSGSYYWFDPWGQGTLVTVSS
D08-VL
(SEQ ID NO: 31)
DIRLTQSPSFLSASVGDRVTITCRASQGISSYLAWYQQKPGKAPKFLIYA
ASTLQSGVPSRFSGSGSGTEFTLTISSLQPEDFATYYCQQLNSYLTFGQG
TRLEIK
B03-VH
(SEQ ID NO: 32)
QMQLVQSGSELKKPGASVKVSCKASGYTFTSYPMNWVRQAPGQGLEWMGW
INTNTGNPTYAQGFTGRFVFSLDTSVSTAYLQISSLKAEDTAVYYCARTG
GYSVDEYSFDIWGQGTTVTVSS
B03-VL
(SEQ ID NO: 33)
DIQLTQSPSFLSASVGDRVTITCRASQGISSYLAWYQQKPGKAPKLLIYA
ASTLQSGVPSRFSGSGSGTEFTLTISSLQPEDFATYYCHQTIFRPTFGGG
TKVEIK
G05-VH
(SEQ ID NO: 34)
QVQLVQSGSELKKPGASVKVSCKASGYTFTSYPMNWVRQAPGQGLEWMGW
INTNTGNPTYAQGFTGRFVFSLDTSVSTAYLQISSLKAEDTAVYYCARVG
GFTVPEYAFDIWGQGTTVTVSS
G05-VL
(SEQ ID NO: 35)
DIRLTQSPSFLSASVGDRVTITCRASQGISSYLAWYQQKPGKAPKLLIYA
ASTLQSGVPSRFSGSGSGTEFTLTISSLQPEDFATYYCGQVMRYPAFGGG
TKVEIK

3.2 Selectivity

3963H03, 3964A06, 3965D08, 3966C11 and derivatives had no detectable binding to the related family member protein CD226 or the unrelated protein PD-L1 in ELISA EC50 assays.

For a more comprehensive evaluation of selectivity, a proprietary fixed-cell microarray technology was used by Retrogenix Ltd. (High Peak, UK) to screen variant 3963H03-12 for off-target binding to a library of 5647 human proteins, comprised predominantly of cell-surface membrane proteins. The library included most known immunoglobulin superfamily receptors that are related to TIGIT such as CD226, CD96, PVR, and NECTINs 1-4. The study was done in four phases: (1) a prescreen to determine background levels and the optimal test antibody concentration for screening, (2) the primary screen for 3963H03-12 binding to fixed HEK293 cells expressing 5647 proteins, (3) a confirmation/specificity screen done by re-expressing putative hits in HEK293 cells and testing binding of 3963H03-12 to the fixed cells along with an isotype control, and (4) further validation by expressing the specific hits in live HEK293 cells and analyzing binding to both 3963H03-12 and the isotype control by flow cytometry.

Eleven binders were identified in the primary screen with intensities ranging from very weak to strong. All eleven were confirmed as binders in the confirmation/specificity secondary screen. The strong binders included the 3963H03-12 target protein TIGIT. Six of the eleven primary binders were also bound by one of the control antibodies and were classified as non-specific binders. These included Fc gamma receptors that either bind the primary antibody Fc or the secondary antibody directly. One binder was very weak and too close to background to consider significant, leaving the four final binders: TIGIT (Genbank accession NM_173799.3), TMEM25 isoform 1 (Genbank accession NM_032780.3), HAVCR2 (Genbank accession BC063431.1), and Cyclin G Associated Kinase (GAK, Genbank accession B0008668). GAK is an intracellular protein that did not bind 3963H03-12 when expressed in live HEK293 cells, and thus was invalidated. TMEM25 isoform 1 and HAVCR2 are transmembrane proteins and were scored as weak binders to 3963H03-12 in the fixed cell screens, then subsequently shown to have low level interactions on live transfected cells with 4.3-fold and 3.0-fold higher median fluorescence than 3963H03-12 binding to vector-only transfected HEK293 cells. The isotype control antibody had binding to TMEM25 isoform 1 and HAVCR2 transfected HEK293 cells slightly lower than 3963H03-12, with 1.4-fold and 1.9-fold higher median fluorescence than isotype control binding to vector-only transfected HEK293 cells, which was similar to the binding to cells transfected with the intracellular protein GAK. In contrast, 3963H03-12 demonstrated 130-fold higher median fluorescence for binding to TIGIT-transfected HEK293 cells compared to vector-only transfected HEK293 cells. Altogether, these results show 3963H03-12 binds selectively to TIGIT.

3.3 ELISA Based TIGIT:CD155 Competition Assay

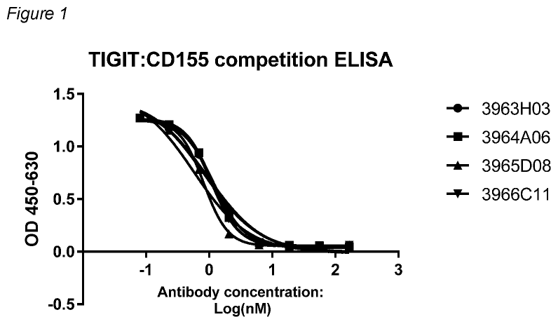

The ability of anti-TIGIT antibodies and a control antibody to compete with the binding of biotinylated human TIGIT-Fc chimera to human CD155-Fc chimera was determined by a competitive ELISA. FIG. 1 shows representative competition curves for the test antibodies. The results demonstrated that the anti-TIGIT antibodies 3963H03, 3964A06, 3965D08, 3966C11 efficiently block the interaction of TIGIT and CD155 with IC50 s of 0.8-1.2 nM.

The following assay protocol was used:

1. 96-well plates were coated with 2.5 μg/ml rhCD155-Fc (Sino Biologicals; Cat #10109H02H) at 50 μl/well and incubated overnight at 4° C.

2. Rinsed wells 3 times with PBS, 0.05% Tween, 200 μl/well.

3. Blocked wells with 200 μl of 1% BSA in PBS, for 1 h at room temperature.

4. Rinsed wells 3 times with PBS, 0.05% Tween, 200 μl/well.

5. Mixed 75 μl 1 mg/ml human TIGIT-Fc-biotin (R&D Systems cat. No. 7898-TG biotinylated at EMD-Serono) with 75 μl test or control antibodies in a 1:3 dilution series (166.7 to 0.08 nM) and added 50 μl each to duplicate wells. Incubated 2 hours at room temperature.

6. Rinsed wells 3 times with PBS, 0.05% Tween, 200 μl/well.

7. For detection streptavidin-HRP conjugate (Millipore; Cat #18-152) was added, 100 μl/well at a 1:200 dilution; and incubated 30 min at room temperature.

8. Rinsed wells 3 times with PBS, 0.05% Tween, 200 μl/well.

9. Added 1-Step™ Ultra TMB-ELISA Substrate Solution (ThermoFisher Scientific Cat #34028), 100 μl/well and incubated 1-2 min at room temperature.

10. Added 100 μl 2N sulfuric acid to each well.

11. Measured ODs at 450 nm and 630 nm on ELISA plate reader.

3.4 Structural and Functional TIGIT Epitope Mapping

a). Co-Crystallization of TIGIT with Fab Fragments of the Present Invention

Crystal structures of the complex of human TIGIT ECD and various Fab fragments of the antibodies in the present invention were determined to identify the interacting amino acids between human TIGIT and the antibody variable region. Human TIGIT was expressed in E. coli inclusion bodies, refolded, and purified by affinity and size exclusion chromatography. The Fab fragments were expressed with His-tags in Expi293F cells and purified by affinity chromatography according to standard methods. The 1:1 complex of TIGIT and each Fab fragment was formed and purified by gel filtration chromatography yielding a homogenous protein complex with a purity greater than 95%. The solution containing the complex was concentrated and standard techniques of high throughput vapor diffusion crystallization screening were applied.

Crystals of the 3963H03 Fab in complex with human TIGIT were grown by mixing 0.75 μl protein solution (50 mM Tris-HCl pH 7.5, 200 mM NaCl, at 24.57 mg/mL) with 0.5 μl reservoir solution (0.2 M Ammonium citrate pH 7.0, 20% PEG 3350) and 0.25 μl seed stock at 20° C. using sitting drop vapor diffusion method. Crystals of 3963H03-12 Fab in complex with human TIGIT were grown by mixing 0.5 μl protein solution (50 mM Tris-HCl pH 7.5, 200 mM NaCl, at 20.26 mg/mL) with 0.3 μl reservoir solution (0.15 M sodium citrate, 0.1 M Bis-Tris 8.5, 22% PEG 3350) and 0.2 μl seed stock at 20° C. using sitting drop vapor diffusion method. Crystals of 3964A06 Fab in complex with human TIGIT were grown by mixing 0.75 μl protein solution (50 mM Tris-HCl pH 7.5, 200 mM NaCl, at 18.66 mg/mL) with 0.5 μl reservoirs solution (0.2 M Sodium Formate, 20% PEG 3350) and 0.25 μl seed stock at 20° C. using sitting drop vapor diffusion method. Crystals of 3966C11 Fab in complex with human TIGIT were grown by mixing 0.5 μl protein solution (50 mM Tris-HCl pH 7.5, 200 mM NaCl, at 27 mg/mL) with 0.5 μl reservoirs solution (16% PEG 4000, 5%-10% Isopropanol, 0.1 M Hepes pH 7.5) at 20° C. using sitting drop vapor diffusion method. Crystals of 7728B03 Fab in complex with human TIGIT were grown by mixing 0.75 μl protein solution (50 mM Tris-HCl pH 7.5, 200 mM NaCl, at 22.35 mg/mL) with 0.5 μl reservoirs solution (25% PEG 3350, 0.1M Tris pH 8.5) and 0.25 μl seed stock at 20° C. using sitting drop vapor diffusion method. Crystals of 7729G05 Fab in complex with human TIGIT were grown by mixing 0.3 μl protein solution (50 mM Tris-HCl pH 7.5, 200 mM NaCl, at 21 mg/mL) with 0.2 μl reservoirs solution (0.1 M Phosphate/citrate, 40% v/v Ethanol, 5% w/v PEG 1000) and 0.1 μl seed stock at 20° C. using sitting drop vapor diffusion method.

Crystals were flash-frozen and measured at a temperature of 100 K. The X-ray diffraction data have been collected at the SWISS LIGHT SOURCE (SLS, Villigen, Switzerland) or at the Deutsches Elektronen-Synchrotron (Hamburg, Germany) using cryogenic conditions. Data were processed using the programmes XDS.

The structure of the complex was solved by molecular replacement using Phaser, version 2.5.7 (McCoy, A. J. et al. J. Appl. Cryst. (2007). 40, 658-674) using structures of human TIGIT (PDB ID: 3UCR) and a Fab (in-house structure) as search models. The structure was refined using Buster, version 2.11.6 (Bricogne, G. et al. Buster version 2.11.6 (2016) Cambridge, United Kingdom: Global Phasing Ltd.). All models including the final protein model were built using COOT version 0.8.1 (Emsley, P. & Cowtan, K. (2004). Acta Cryst. D60, 2126-2132). All relevant data regarding data collection, data processing, structure refinement and structure quality can be found in Table 7 and Table 8.

TABLE 7
Data collection and processing
statistics for TIGIT complexed with Fabs

| | | TIGIT:3963H03- | |
|---|---|---|---|
| | TIGIT:3963H03 | 12 | TIGIT:3964A06 |
| X-ray Source | PXI/X06SA | PX11 (DESY2) | PXII/X10SA |
| | (SLS1) | | (SLS1) |
| Wavelength | 0.99998 | 1.033227 | 1.000000 |
| [Å] | | | |
| Detector | Pilatus 6M | Pilatus 6M | Pilatus 6M |
| Temperature | 100 | 100 | 100 |
| [K] | | | |
| Space Group | P41212 | P41212 | C121 |
| Cell: a; b; c; | 109.39 | 107.96; | 121.96; |
| [Å] | 109.39 | 107.96; | 80.79; |
| | 230.74 | 231.50 | 126.86 |
| α; β; γ; [°] | 90.0; | 90.0; | 90.0 |
| | 90.0; | 90.0; | 108.57 |
| | 90.0 | 90.0 | 90.0 |
| Resolution [Å] | 2.41 (2.55-2.41)3 | 2.87 (2.92-2.87) | 1.55 (1.55-1.64) |
| Unique | 53945 | 32153 | 167333 |
| reflections | | | |
| Multiplicity | 9.9 (7.7) | 25.6 (27.4) | 3.35 (3.16) |
| Completeness | 97.9 (88.9) | 100 (99.8) | 98.8 (97.0) |
| [%] | | | |
| Rsym [%]4 | 15.6 (132) | 2.6 (33.4) | 6.3 (110) |
| Rmeas [%]5 | 16.4 (140) | 13.2 (176) | 6.4 (117) |
| Mean(I)/sd6 | 11.6 (0.9) | 22.7 (2.3) | 11.5 (0.97) |
| | TIGIT:3966C11 | TIGIT:7728B03 | TIGIT:7729G05 |
| X-ray Source | PXII/X10SA | PXIII/X06DA | PXI/X06SA |
| | (SLS1) | (SLS1) | (SLS1) |
| Wavelength | 1.000020 | 1.000000 | 0.999930 |
| [Å] | | | |
| Detector | Pilatus 6M | Pilatus 6M | Pilatus 6M |
| Temperature | 100 | 100 | 100 |
| [K] | | | |
| Space Group | C121 | P212121 | C121 |
| Cell: a; b; c; | 118.77 | 65.23 | 116.64 |
| [Å] | 87.96 | 73.00 | 68.29 |
| | 126.86 | 117.98 | 262.66 |
| α; β; γ; [°] | 90.0 | 90.0 | 90.0 |
| | 102.57 | 90.0 | 90.13 |
| | 90.0 | 90.0 | 90.0 |
| Resolution [Å] | 1.73 (1.84-1.73) | 1.35 (1.43-1.35) | 1.9 (2.02-1.9) |
| Unique | 131029 | 123912 | 160020 |
| reflections | | | |
| Multiplicity | 3.33 (3.18) | 6.45 (6.17) | 3.17 (3.08) |
| Completeness | 98.7 (94.2) | 99.8 (99.1) | 98.2 (95.0) |
| [%] | | | |
| Rsym [%]3 | 5.7 (143) | 5.1 (200) | 9.9 (90.5) |
| Rmeas [%]4 | 5.8 (164) | 4.6 (225) | 10.6 (130) |
| Mean(I)/sd5 | 11.3 (0.7) | 17.6 (0.95) | 6.9 (0.86) |

1SWISS LIGHT SOURCE (SLS, Villigen, Switzerland)
2values in parenthesis refer to the highest resolution
$\begin{matrix}3 Rsym = \frac{{\sum }_{h} {\sum }_{i}^{{n}_{h}} \vert {\hat{I}}_{h} - {I}_{h , i} \vert}{{\sum }_{h} {\sum }_{i}^{{n}_{h}} {I}_{h , i}} with {\hat{I}}_{h} = \frac{1}{{n}_{h}} {\sum }_{i}^{{n}_{h}} {I}_{h , i} where {I}_{h , i} is the \\ intensity value of the ith measurement of h\end{matrix}$ $\begin{matrix}4 Rmeas = \frac{{\sum }_{h} \sqrt{\frac{{n}_{h}}{{n}_{h} - 1}} {\sum }_{i}^{{n}_{h}} \vert {\hat{I}}_{h} - {I}_{h , i} \vert}{{\sum }_{h} {\sum }_{i}^{{n}_{h}} {I}_{h , i}} with {\hat{I}}_{h} = \frac{1}{{n}_{h}} {\sum }_{i}^{{n}_{h}} {I}_{h , i} where {I}_{h , i} \\ is the intensity value of the ith measurement of h\end{matrix}$ 5calculated with independent reflections
1SWISS LIGHT SOURCE (SLS, Villigen, Switzerland)
2Deutsches Elektronen-Synchrotron (Hamburg, Germany)
3values in parenthesis refer to the highest resolution
$\begin{matrix}4 Rsym = \frac{{\sum }_{h} {\sum }_{i}^{{n}_{h}} \vert {\hat{I}}_{h} - {I}_{h , i} \vert}{{\sum }_{h} {\sum }_{i}^{{n}_{h}} {I}_{h , i}} with {\hat{I}}_{h} = \frac{1}{{n}_{h}} {\sum }_{i}^{{n}_{h}} {I}_{h , i} where {I}_{h , i} is the \\ intensity value of the ith measurement of h\end{matrix}$ $\begin{matrix}5 Rmeas = \frac{{\sum }_{h} \sqrt{\frac{{n}_{h}}{{n}_{h} - 1}} {\sum }_{i}^{{n}_{h}} \vert {\hat{I}}_{h} - {I}_{h , i} \vert}{{\sum }_{h} {\sum }_{i}^{{n}_{h}} {I}_{h , i}} with {\hat{I}}_{h} = \frac{1}{{n}_{h}} {\sum }_{i}^{{n}_{h}} {I}_{h , i} where {I}_{h , i} \\ is the intensity value of the ith measurement of h\end{matrix}$ 6calculated with independent reflections

TABLE 8
Refinement Statistics for TIGIT¹

| | | TIGIT:3963H03- | |
|---|---|---|---|
| | TIGIT:3963H03 | 12 | TIGIT:3964A06 |
| Resolution [Å] | 48.92-2.41 | 97.84-2.87 | 46-62-1.55 |
| Number of reflections | 53945/2698 | 32072/1677 | 167299/8365 |
| (working/test) | | | |
| Rcryst [%] | 0.187 | 0.195 | 0.174 |
| Rfree [%]2 | 0.228 | 0.231 | 0.202 |
| DPI [σ(x, B_{avg})]3 | 0.218 | 0.322 | 0.075 |
| Total number of atoms: | | | |
| Protein | 8221 | 7194 | 8039 |
| Water | 194 | — | 1318 |
| Deviation from ideal | | | |
| geometry:4 | | | |
| Bond lengths [Å] | 0.010 | 0.009 | 0.01 |
| Bond angles [°] | 1.29 | 1.15 | 1.11 |
| Ramachandran plot:5 | | | |
| Most favored regions [%] | 94.7 | 95.5 | 97.9 |
| Additional allowed regions | 4.5 | 3.5 | 2.1 |
| [%] | | | |
| Disallowed regions [%] | 0.8 | 1 | 0 |
| | TIGIT:3966C11 | TIGIT:7728B03 | TIGIT:7729G05 |
| Resolution [Å] | 48.59-1.73 | 48.64-1.35 | 38.00-1.90 |
| Number of reflections | 131021/6552 | 123904/6196 | 157863/7715 |
| (working/test) | | | |
| Rcryst [%] | 0.202 | 0.184 | 0.209 |
| Rfree [%]2 | 0.227 | 0.205 | 0.239 |
| DPI [σ(x, B_{avg})]3 | 0.103 | 0.055 | 0.135 |
| Total number of atoms: | | | |
| Protein | 8183 | 4092 | 12255 |
| Water | 823 | 716 | 1334 |
| Deviation from ideal | | | |
| geometry:4 | | | |
| Bond lengths [Å] | 0.010 | 0.010 | 0.01 |
| Bond angles [°] | 1.11 | 1.15 | 1.2 |
| Ramachandran plot:5 | | | |
| Most favored regions [%] | 97.4 | 97.4 | 96.4 |
| Additional allowed regions | 2.6 | 2.6 | 3.3 |
| [%] | | | |
| Disallowed regions [%] | 0 | 0 | 0.3 |

¹Values as defined by REFMAC5, without sigma cut-off
2Test-set contains 5% of measured reflections
3Diffraction-component precision index (DPI) is calculated according to Eq. 27 of CRUICKSHANK. D.W.J. (1999) ACTA CRYST D55, 583-601, where σ(x, B_{avg}) = 1.0(Nᵢ/n_{obs})^{1/2}C^{-1/3}R_{free}dₘᵢₙ
4Root mean square deviations from geometric target values
5Calculated with MOLPROBITY

The structures of the Fab format of anti-TIGIT antibodies 3963H03, 3963H03-12, 3966C11, 3964A06, 7729G05 and 7728B03 in complex with the TIGIT ECD were solved with resolution of 2.41, 2.87, 1.73, 1.6, 1.9, and 1.35 Å respectively.

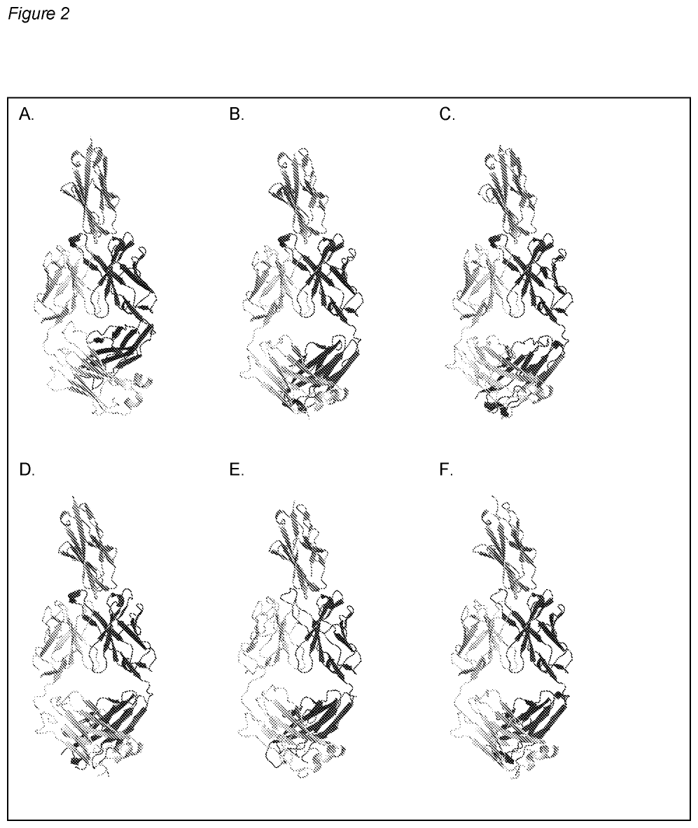

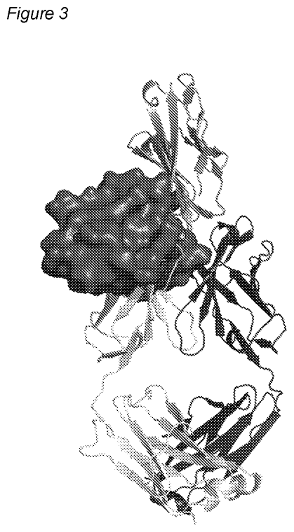

The complexes have nearly identical folding, as displayed in FIG. 2 with average RMSD of 0.79 Å on non-hydrogen atoms of the antigen and antibody variable region. The structures show that each Fab binds to a region on TIGIT that will sterically interfere with PVR binding. Indeed, FIG. 3 provides an overlay of the TIGIT:3963H03-12 co-crystal structure with the available TIGIT:PVR co-crystal structure (Protein Data Bank entry 3UDW) showing significant overlap of PVR and the Fab.

The crystal structures of human TIGIT ECD with anti-TIGIT Fab complexes were used to identify the epitope of the anti-TIGIT Fabs on TIGIT. Contact residues are defined as residues of TIGIT with a non-hydrogen atom within 3.8 angstroms of a non-hydrogen atom of the Fab. Distances were measured from the final crystallographic coordinates using the BioPython package. Contacts present in all complexes of the asymmetric unit of each crystal structure are reported in Table 9. The interaction surface on TIGIT by the Fabs was formed by several continuous and discontinuous (i.e. noncontiguous) sequences: namely residues Met23, Thr51, Ala52, Gln53, Thr55, Gln56, Asn70, Ala71, Asp72, His111, Thr112, Tyr113, Pro114, Asp115, Gly116, or Thr117 as detailed in Table 9. These residues form the exemplary three-dimensional conformational epitope that is recognized by the anti-TIGIT Fabs described in this invention.

TABLE 9
Contacts present in all complexes of the asymmetric unit of each crystal structure.
Interactions between human TIGIT and antigen binding region of antibodies described in the
invention. The antibody residues are numbered based upon their linear amino acid sequence.
Corresponding chains are labeled (“H” for heavy chain, “L” for light chain). TIGIT residues shown
here have at least one non-hydrogen atom within 3.8 Å to an non-hydrogen atom in the antibody.

| TIGIT | 3963H03 | 3963H03-12 | 3966C11 | 3964A06 | 7729G05 | 7728B03 |
|---|---|---|---|---|---|---|
| Met23 | | | | | Arg93.L | |
| Thr51 | Trp50.H | Trp50.H | Trp50.H | Trp50.H | Trp50.H | Trp50.H |
| | Asn52.H | Asn52.H | Asn52.H | Asn52.H | Asn52.H | Asn52.H |
| | Asn57.H | | Asn57.H | Asn57.H | | Asn57.H |
| | | | Thr59.H | | | |
| Ala52 | Asn52.H | Asn52.H | Asn52.H | Asn52.H | | Asn52.H |
| Gln53 | Thr30.H | Thr30.H | Thr30.H | Thr30.H | Thr30.H | Thr30.H |
| | | | | | Ser31.H | |
| | Pro33.H | Pro33.H | Pro33.H | Pro33.H | Pro33.H | Pro33.H |
| | Trp50.H | Trp50.H | Trp50.H | Trp50.H | Trp50.H | Trp50.H |
| | Asn52.H | Asn52.H | Asn52.H | Asn52.H | Asn52.H | Asn52.H |
| | Thr53.H | Thr53.H | Thr53.H | Thr53.H | Thr53.H | Thr53.H |
| | Asn54.H | Asn54.H | Asn54.H | Asn54.H | Asn54.H | Asn54.H |
| Thr55 | Tyr102.H | Tyr102.H | Tyr102.H | Tyr102.H | Phe102.H | Tyr102.H |
| | Tyr107.H | Tyr107.H | Tyr107.H | Tyr107.H | Tyr107.H | Tyr107.H |
| Gln56 | Tyr102.H | Tyr102.H | Tyr102.H | Tyr102.H | Phe102.H | Tyr102.H |
| | | | | Ser103.H | Thr103.H | |
| Asn70 | | Tyr102.H | Tyr102.H | Tyr102.H | Phe102.H | Tyr102.H |
| Ala71 | | | | | Ser31.H | Ser31.H |
| | Tyr32.H | | Tyr32.H | | | |
| | | | | | Phe102.H | |
| Asp72 | | | | | | Thr28.H |
| | | | | | | Ser31.H |
| | | | | | | Tyr32.H |
| | Tyr102.H | Tyr102.H | Tyr102.H | Tyr102.H | | |
| His111 | Tyr102.H | Tyr102.H | Tyr102.H | Tyr102.H | Phe102.H | Tyr102.H |
| | Ser103.H | Ser103.H | Ser103.H | Ser103.H | Thr103.H | Ser103.H |
| | Val104.H | Val104.H | Val104.H | Val104.H | Val104.H | Val104.H |
| | Tyr107.H | Tyr107.H | Tyr107.H | Tyr107.H | Tyr107.H | Tyr107.H |
| Thr112 | Tyr107.H | Tyr107.H | Tyr107.H | Tyr107.H | Tyr107.H | Tyr107.H |
| Tyr113 | Pro33.H | Pro33.H | Pro33.H | Pro33.H | Pro33.H | Pro33.H |
| | Asn35.H | Asn35.H | Asn35.H | Asn35.H | Asn35.H | Asn35.H |
| | | | | | | Trp47.H |
| | Trp50.H | Trp50.H | Trp50.H | Trp50.H | Trp50.H | Trp50.H |
| | | | | | Val91.L | Thr91.L |
| | Pro95.L | Pro95.L | Pro95.L | Pro95.L | Pro95.L | |
| | Val99.H | Val99.H | Val99.H | Val99.H | Val99.H | Thr99.H |
| | Tyr107.H | Tyr107.H | Tyr107.H | Tyr107.H | Tyr107.H | Tyr107.H |
| | Phe109.H | Phe109.H | Phe109.H | Phe109.H | Phe109.H | Phe109.H |
| Pro114 | Trp50.H | Trp50.H | Trp50.H | Trp50.H | Trp50.H | Trp50.H |
| | | | | | Val91.L | |
| | | | | | Met92.L | Ile92.L |
| | Ser93.L | Ser93.L | | | Arg93.L | Phe93.L |
| Asp115 | | | | | Arg93.L | Phe93.L |
| Gly116 | | | | | | Ile92.L |
| | Val104.H | Val104.H | Val104.H | Val104.H | Val104.H | Val104.H |
| Thr117 | Val104.H | | | Val104.H | Val104.H | Val104.H |

b) Mutagenesis

The contribution to anti-TIGIT antibody binding energy for contact residues on the TIGIT ECD was assessed by mutation of selected residues to alanine. Positions where the parental residue was alanine or proline were replaced with glycine. The loss of binding energy upon mutation indicates the importance of the parental residue to binding. In total, 11 human TIGIT variants with a point mutation to alanine or glycine were designed. The mutants were expressed in E. coli and purified with affinity and size exclusion chromatography. Binding kinetics to antibody 39631H03-12 were characterized using surface plasmon resonance (SPR). Binding hotspots, or residues that contribute most to the binding energy (Wells. J. A., PNAS 93, 1-6, 1996), were identified as those that did not meet a threshold binding signal at 100 nM antigen. Furthermore, the affinity of the antibody for wild-type and each mutant was determined and used to calculate the contribution of each epitope residue to the binding energy.

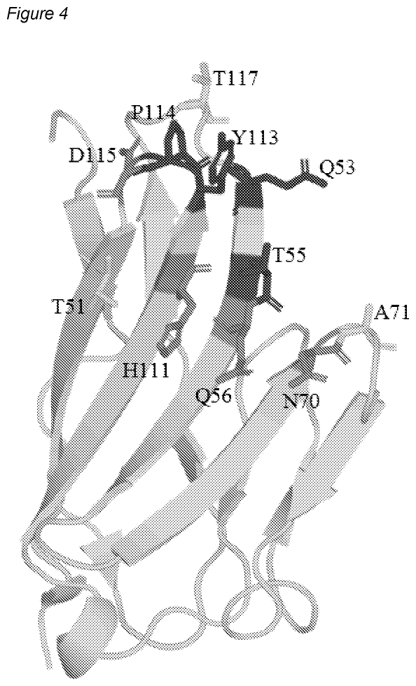

A diagram of the TIGIT ECD structure with the mutagenized residues shown in sticks, and shaded according to the change in affinity, is shown in FIG. 4. In addition, the results are summarized in Table 10 below, where 11 point mutants of TIGIT were compared to wild-type TIGIT antigen for antibody binding. SPR (Biacore) was used to perform a kinetic study allowing determination of kinetic rate constants (kₐ and k_{d}). Briefly, goat polyclonal anti-human Fc antibody was chemically coupled to a CM5 chip. 3963H03-12 antibody was injected next and captured by the polyclonal antibody. Buffer was used to wash out unbound antibody until the baseline RU stabilized. Antigen (wild-type or mutant human TIGIT ECD) was next injected at a fixed concentration for 3 minutes and the association was recorded. Buffer was injected for a further 3 minutes and dissociation was observed. The antigens were injected at concentrations of 100 nM, 50 nM, 25 nM, 12.5 nM and 6.25 nM. Between each cycle, the chip was regenerated with low pH buffer and fresh 3963H03-12 was captured prior to injecting the next concentration of antigen. The rate constants were determined by iterative fitting of the data to a 1:1 binding model by an algorithm that minimizes Chi-squared. The equilibrium dissociation constant (K_{D}) was calculated as the ratio of the kinetic constants and the change in the Gibbs free energy of binding of mutant relative to wild-type TIGIT (ΔΔGₘᵤₜ) was derived from the ratio of the wild-type and mutant K_{D}'s. The free energy changes are highlighted according to destabilization of antibody-antigen binding; “**”: >3 kcal/mol destabilization (binding hotspots); “*”: >0.7 kcal/mol. According to this analysis, amino acids marked with “**” or “*” are part of the functional epitope. NB denotes no binding. The temperature midpoint of fluorescently monitored thermal denaturation is given for the wild type and mutant proteins. The wild type TIGIT and all its mutants show monodisperse on size exclusion chromatography (SEC). For K_{D}, the mean and standard deviation is given where n>1.

TABLE 10
Epitope mapping through analysis of 3963H03-12 binding affinity
to TIGIT mutants

| | ΔΔGₘᵤₜ | | |
|---|---|---|---|
| Mutation | (kcal/mol) | K_{D} (nM) | T_{1/2} (° C.) |
| huTIGIT | 0.00 | 2.08 +/− 0.11 | 59.0 |
| T51A | −0.10 | 1.77 +/− 0.55 | 54.0 |
| Q53A | >3** | NB | 54.7 |
| T55A | >3** | NB | 52.3 |
| Q56A | 1.65* | 33.90 +/− 1.23 | 50.7 |
| N70A | 1.61* | 31.47 +/− 8.73 | 53.4 |
| A71G | 0.72* | 7.02 +/− 0.62 | 54.4 |
| H111A | 1.60* | 30.90 +/− 0.30 | 48.2 |
| Y113A | >3** | NB | 53.0 |
| P114G | >3** | NB | 51.8 |
| D115A | 1.14* | 14.38 +/− 1.06 | 51.5 |
| T117A | −0.17 | 1.58 +/− 0.33 | 52.7 |

It was important to confirm that the lack of binding to 3963H03-12 of the Q53A, T55A, Y113A and P114G point mutants was indeed due to loss of hotspot residues and not to global unfolding of the antigen. The structural integrity of the mutated proteins was confirmed using a fluorescence monitored thermal unfolding assay in which the protein is incubated with a dye that is quenched in aqueous solution but fluoresces when bound by exposed hydrophobic residues. As the temperature increases, thermal denaturation of the protein exposes the hydrophobic core residues, and this can be monitored by an increase in fluorescence of the dye. The data were fit to equation 2 (adapted from Bullock, A. N. et al. Thermodynamic stability of wild-type and mutant p53 core domain. PNAS 94, 14338-14342 (1997)) to determine the temperature at the inflection point of the curve (T_{1/2}).

$$F = \frac{\{ Fi + β i * T + ( ( Fmax + β max * T ) * {e}^{[ m * ( T - T 1 / 2 ) ]} ) \}}{1 + {e}^{[ m * ( T - T 1 / 2 ) ]}} \qquad Equation 2$$

Mutants of Q53A, T55A, Y113A and P114G displayed minimal destabilization of the antigen indicated by a small decrease in the T_{1/2} of fluorescence monitored unfolding (Table 10). This confirms Q53, T55, Y113 and P114 are true binding hotspots for 3963H03-12. The structural integrity of most of the other mutant proteins was also confirmed by this method (Table 10). The observation that most mutant proteins behaved similarly to wild type on analytical size exclusion chromatography provides further support for native structure of mutant antigen proteins.

3.5 EC50 Measured by Direct FACS Binding Assay

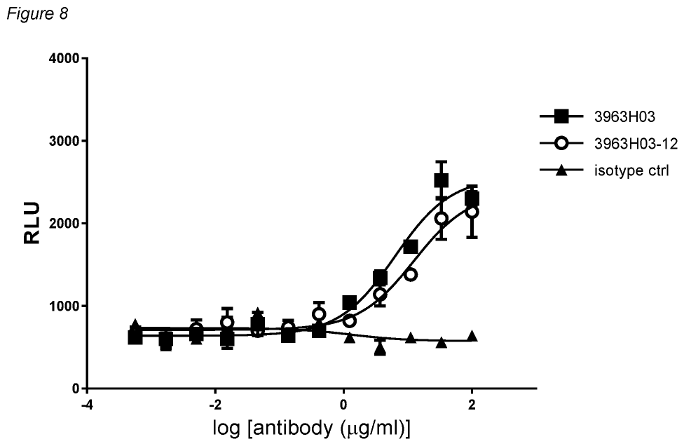

The dose dependent binding ability of 3963H03 to the target on the cell surface was confirmed by flow cytometry. It efficiently binds to human TIGIT ECD expressed on the CHO-S cell surface with an EC50 of 4.7 nM and to cynomolgus monkey TIGIT ECD expressed on the CHO-S cell surface with an EC50 of 3.6 nM (Table 11 and FIG. 8). The assays qualitatively described the dose dependent binding characteristics of the anti-TIGIT antibody.

TABLE 11
EC50 binding of anti-TIGIT antibody to cells expressing human
TIGIT ECD or cyno TIGIT ECD measured by flow cytometry

| | Cell binding EC50 (nM) | |
|---|---|---|
| Antibody | CHO-S-hTIGIT | CHO-S-cynoTIGIT |
| 3963H03 | 4.7 | 3.6 |
| Isotype control | NA | NA |

3.6 TIGIT Jurkat Reporter Assay

3963H03 along with its sequence optimized variant, 3963H03-12, were tested in a cell based TIGIT/CD155 Blockade Bioassay (Promega Cat. No. CS198801) using the protocol supplied by the manufacturer. The assay is comprised of human Jurkat cells expressing recombinant human TIGIT with a luciferase reporter gene driven by the IL2 promoter, co-cultured with CHO-K1 cells expressing human CD155 and a T-cell receptor activator. The B-cell cloning hit 3963H03 and its sequence optimized variant 3963H03-12, formatted with IgG1 and kappa constant regions, had similar EC50 s ranging from 6.3 to 12.5 ug/ml (FIG. 8 and Table 12).

TABLE 12
Cell-based TIGIT/CD155 Blockade assay with sequence optimized
and affinity matured anti-TIGIT antibody variants

| | EC50 (ug/ml) | Max RLU (potency) |
|---|---|---|
| 3963H03 | 6.296 | 2560 |
| 3963H03-12 | 12.5 | 2384 |
| Isotype control | No activity | 575 |

3.7 Antibody Dependent Cell-Mediated Cytotoxicity (ADCC)

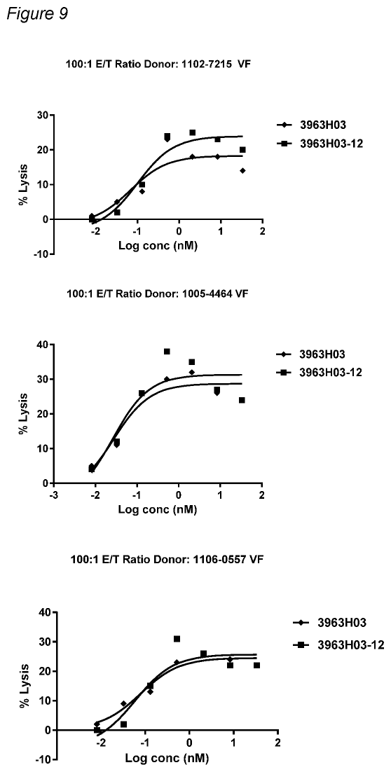

The ADCC activities of anti-TIGIT 3963H03 and its sequence optimized variant 3963H03-12 were tested using stably transfected CHO-S-hTIGIT target cells and donor effector cells with heterozygous FcγRIIIa 158V/158F allotype using standard Chromium release assay. Briefly, CHO-S-hTIGIT cells were first labeled with ⁵¹Cr for 45 min, then incubated for 15 min at 37° C. with 4-fold serial dilutions of anti-TIGIT antibodies at the starting concentration of 33 nM. Effector cells were added at the ratio of 1:100 and incubated for 4 hours at 37° C. Cells were transferred to Lumaplate 96 well DryPlates, dried overnight and radioactivity was measured using a gamma counter. The percent lysis was calculated as the ratio of ((Count-Spont)/(100% Lysis-Spont))×100 where Spont is the radioactivity counted with the CHO-S-hTIGIT cells alone (in the absence of antibody and effector cells) and 100% lysis was calculated by lysing the CHO-S-hTIGIT cells with detergent. The example assay shown was performed with effector cells from three donors with the allotype V/F. Both antibodies tested in this example assay induced ADCC of the CHO-S-hTIGIT target cells, with EC50 ranging from 0.026 to 0.1 nM (Table 13) and a similar percent maximal cell lysis of approximately 20-30% (FIG. 9).

TABLE 13
ADCC activity of anti-TIGIT antibodies, EC50 (nM)

| | EC50, nM | | |
|---|---|---|---|
| Antibody | Donor 1102-7215 | Donor 1005-4464 | Donor 1106-0557 |
| 3963H03 | 0.069 | 0.028 | 0.080 |
| 3963H03-12 | 0.100 | 0.026 | 0.059 |

3.8. Complement Dependent Cytotoxicity (CDC) Activity

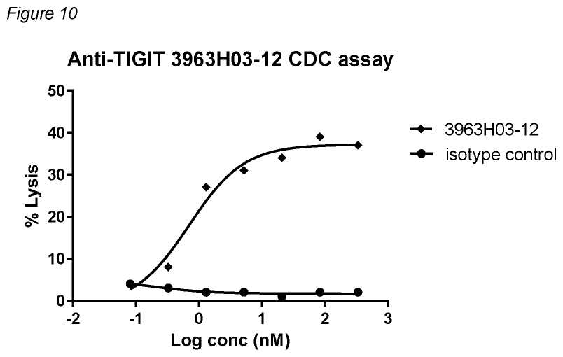

For CDC assay, CHO-S-human TIGIT ECD cells were first labeled with ⁵¹Cr for 45 min, then incubated for 15 min at 37° C. with 4-fold serial dilutions of anti-TIGIT antibodies at the starting concentration of 20,000 ng/ml. Previously CDC-qualified normal human serum complement was added at 1:10 dilution and incubated for 2 hours at 37° C. Cells were transferred to Lumaplate 96-well counting plates, dried overnight and radioactivity was measured using MicroBeta2 counter (Perkin Elmer). The percent lysis was calculated as the ratio of ((Count-Spont)/(100% Lysis-Spont))×100 where Spont is the radioactivity counted with the CHO-S-huTIGIT cells alone (in the absence of antibody and complement) and 100% lysis was calculated by lysing the labelled CHO-S-human TIGIT ECD cells with detergent. FIG. 10 shows an assay performed with CHO-S-human TIGIT ECD target cells and 3963H03-12, demonstrating that this antibody is capable of mediating CDC activity.

3.9 T Cell Activation Assay

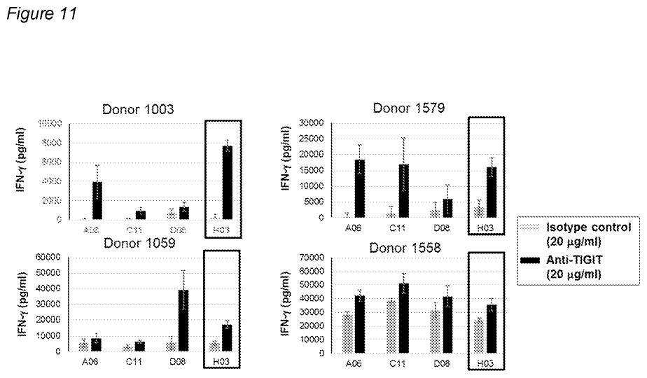

When treated with anti-CD3 and anti-CD28 antibodies, T cells in human PBMCs were activated. Co-treatment of antagonistic anti-TIGIT antibodies could block TIGIT inhibitory signaling and as a result potentially further enhance T cell activation, measured by IFNγ production. Human PBMC were stimulated with 0.5 ng/ml anti-CD3 OKT3 and 20 ng/ml anti-CD28 for 48 hours in the presence of anti-TIGIT antibodies or human IgG1 isotype control (20 μg/ml). IFN-γ in culture supernatant was measured by ELISA. PBMCs from 4 different donors (1003, 1579, 1059, 1558) were tested. Anti-TIGIT antibodies (A06, C11, D08, H03) enhanced IFNγ production as shown in FIG. 11. Anti-TIGIT H03 was shown to more consistently enhance IFNγ production than A06, C11 and D08.

3.10 CD8+ T Cell Antagonistic Assay

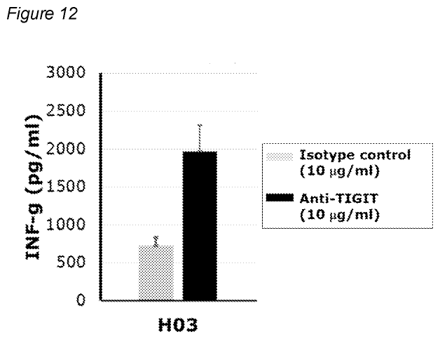

The binding of CD155 to TIGIT triggers inhibitory signaling into CD8+ T cells and co-treatment with an antagonistic anti-TIGIT antibody could block TIGIT/CD155 interaction and as a result enhance T cell activation, measured by IFNγ production. 96-well cell culture plates were co-coated with anti-CD3 (OKT3, 2 μg/ml) and recombinant CD155-Fc (2 μg/ml). Freshly isolated human CD8+ T cells were added and cultured for 4 days in the presence of 10 μg/ml soluble anti-TIGIT antibody or human IgG1 isotype control. IFNγ production in the supernatant was measured by ELISA. Anti-TIGIT 3963H03 reversed CD155-mediated T cell suppression and as a result increased IFNγ production as shown in FIG. 12.

3.11 Primary Cell Binding Assays

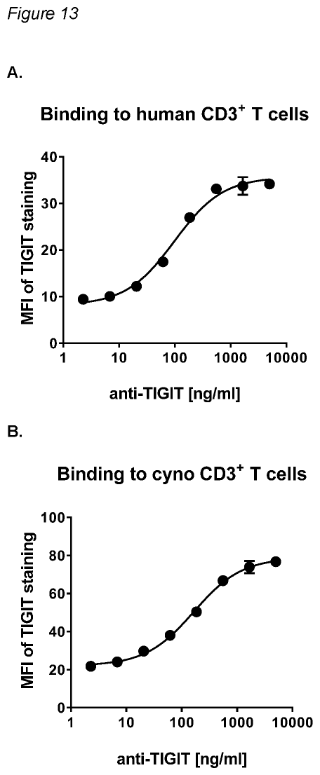

The ability of 3963H03-12 to bind to TIGIT expressed on the surface of human and cynomolgus monkey primary T cells was determined by flow cytometry. Human or cyno PBMCs were incubated with serial dilutions (1:3) of 3963H03-12 and the binding of anti-TIGIT antibody to CD3+ T cells was detected by anti-hIgG APC (1:1000). Flow cytometry analysis was carried out using BD-Calibur. CD3+ T cells were gated, and the mean florescence intensities (MFI) and percent APC staining of the parent population were determined. 3963H03-12 bound to primary human and cynomolgus monkey T cells in a dose-dependent manner with an EC50 of 85.2±28.8 ng/mL (0.6±0.2 nM) and 132.2±29.2 ng/mL (0.8±0.2 nM), respectively, as shown in FIG. 13.

3.12 Target Occupancy (TO) Assays

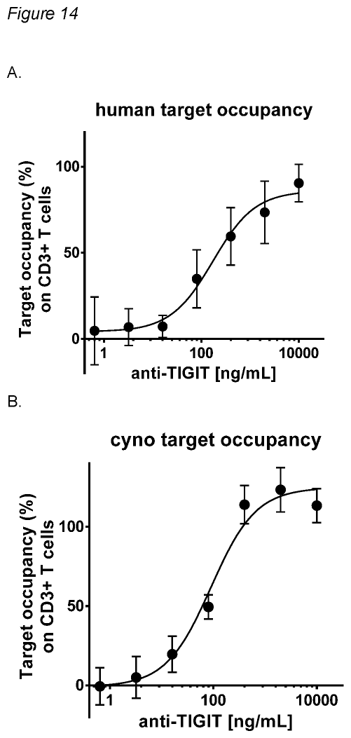

The target occupancy of anti-TIGIT 3963H03-12 on CD3+ T cells was measured via flow cytometry using human whole blood and cynomolgus monkey spleen cells. Serial dilutions of anti-TIGIT were incubated with human or cynomolgus monkey samples for 1 hour, and the unoccupied TIGIT on CD3+ primary T cells was measured by flow cytometry with biotinylated anti-TIGIT (3963H03-12). Flow cytometry analysis was performed using a BD-Calibur gated on CD3+ cells and analyzed as follows. Percentage of target occupancy (TO %) was calculated using the formula, TO (%)=(1−(Dt−Ct)/(D0−C0))*100, where Dt=Percentage of TIGIT staining, Ct=Percentage of isotype control staining at a certain concentration of anti-TIGIT, D0=Percentage of TIGIT staining, and C0=Percentage of isotype control staining in the absence of anti-TIGIT. 3963H03-12 was shown to efficiently saturate target on both human (FIG. 14A) and cynomolgus monkey (FIG. 14B) T cells. The average EC50 from 9 human donors was 239.8±168.04 ng/mL (1.6±1.1 nM) and EC50 from 6 cyno donors was 92.7±21.6 ng/mL (0.6±0.1 nM).

3.13 Cell Based TIGIT/CD155 and TIGIT/CD112 Blocking Assays

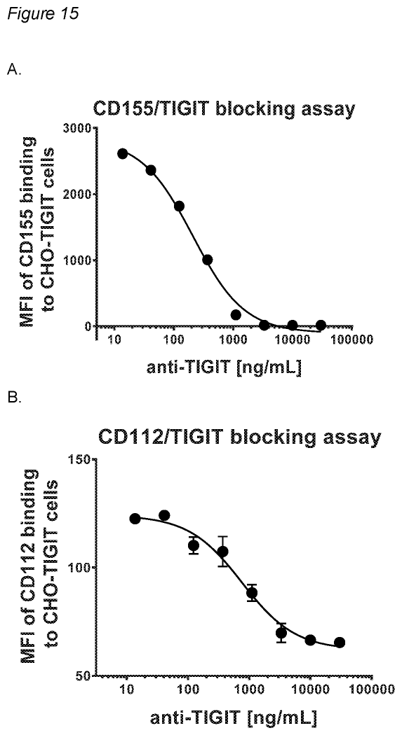

To evaluate the ability of anti-TIGIT 3963H03-12 to block the interaction of TIGIT with its ligands CD155 and CD112, a blocking assay was performed using CHO-S engineered cells stably expressing human TIGIT (CHO-S-hTIGIT cell line #4-60). CHO-S-human TIGIT cells were incubated with serial dilutions (1:3) of 3963H03-12 before biotinylated human CD112-Fc or human CD155-Fc (2 μg/mL final concentration) was added. The interaction of CD155/TIGIT or CD112/TIGIT was detected by streptavidin-APC (1:1000). 3963H03-12 dose-dependently blocked the interaction of TIGIT with CD155 (FIG. 15A) and CD112 (FIG. 15B), with an IC50 of 165.0±39.7 ng/mL (1.1±0.3 nM) and 410.6±315.5 ng/mL (2.8±2.1 nM), respectively.

3.14 Cell Based TIGIT/CD226 Blocking Assay

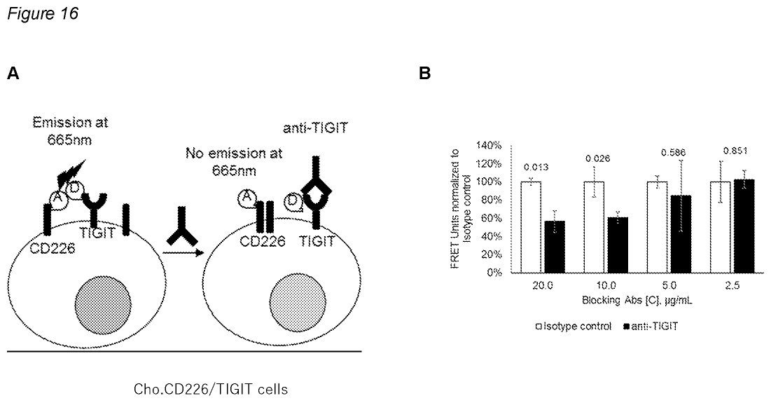

TIGIT receptors expressed on cell surface interact with CD226 and disrupt CD226 homo-dimers that are important for CD226 stimulatory function. Blocking with 3963H03-12 reduces CD226 and TIGIT interactions and potentially leads to increased co-stimulatory signaling by CD226. A FRET assay was designed to measure the interaction between TIGIT and CD226 and the effect of 3963H03-12 on this interaction (FIG. 16A). CHO-CD226 cells were generated by transfection of CHO cells with CD226/SNAP tag plasmid using Lipofectamine 3000 (Invitrogen, L3000-015) and subsequent selection of stably expressing CD226 cells with 250 μg/ml of Hygromycin B (Invitrogen, 10687010). CHO-CD226 cells seeded in white 96-well plates (Greiner Bio-One, 655083) were transfected with 0.1 μg/well TIGIT/HA tag plasmid using Lipofectamine 3000 and incubated with 3963H03-12 or isotype control Abs at concentrations of 10, 1 and 0.1 μg/ml for 24 hours. After that cells were washed with Tag-lite labeling medium (Cisbio, 7SEC30K), then stained with 1 μM SNAP-Red acceptor (Cisbio, SSNPREDE) at 37° C.-5% C02 incubator for 1 hour. Next, cells were washed three times and incubated with 1.6 nM anti-HA-TB cryptate donor (Cisbio, 610HATTA) at room temperature for 2 hours. The FRET signal was recorded at 665 nm and 615 nm for 150 μs after excitation at 320 nm and 60 μs delay using an Envision Plate Reader (Perkin Elmer, Xcite Multilabel Reader). FRET intensity was calculated as (Emission 665 nm/Emission 615 nm from TIGIT-transfected cells)−(Emission 665 nm/Emission 615 nm from mock-transfected cells). Percent of FRET normalized to isotype control was calculated as (FRET intensity for TIGIT-transfected Cho.CD226 cells blocked with anti-TIGIT Abs)/(FRET intensity for TIGIT-transfected Cho.CD226 cells blocked with Isotype control Abs)*100. The quantification of inhibition of FRET signal which measures TIGIT and CD226 interaction by 3963H03-12 demonstrated that 3963H03-12 blocked TIGIT/CD226 interaction (FIG. 16B).

3.15 Allogenic Two-Way MLR (Mixed Lymphocyte Reaction) Assay

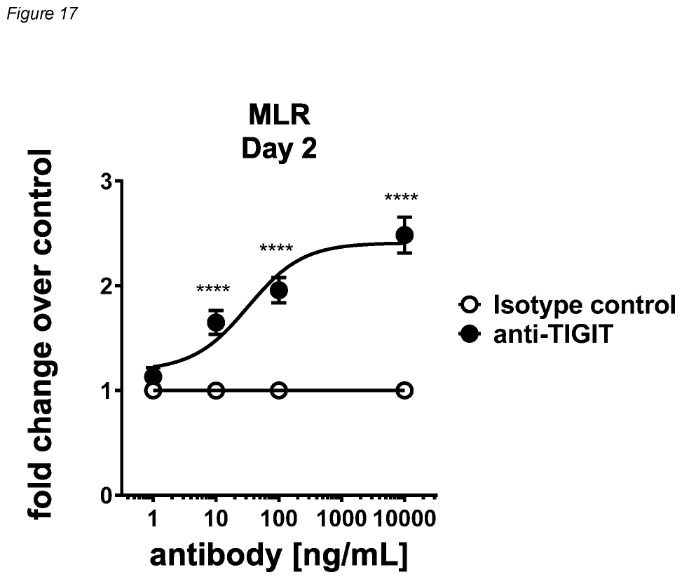

In a two-way MLR assay with PBMCs from two unrelated donors, responder (effector T) undergo activation and proliferation in response to the major histocompatibility antigen (MHC Class I and II) differences between the responder cells and stimulator (target) cells in both donors. Co-treatment with a functional antagonist checkpoint inhibitor (CPI) antibody further potentiates T cell activation, measured by IFNγ production. PBMCs from two different human donors were co-cultured at 1:1 ratio and treated with serial dilutions of 3963H03-12 or isotype control for 2 days. Immune cell activation was evaluated by measuring IFN-γ in the supernatant. Results from 7 assays with 7 different donor pairs were plotted together as fold charges over isotype control at 1 ng/mL which was set to 1. 3963H03-12 dose-dependently enhanced IFN-γ production, with an EC50 of 158.9±185.0 ng/mL (1.1±1.2 nM) (FIG. 17).

3.16 Allogenic One-Way MLR (Mixed Lymphocyte Reaction) Assay

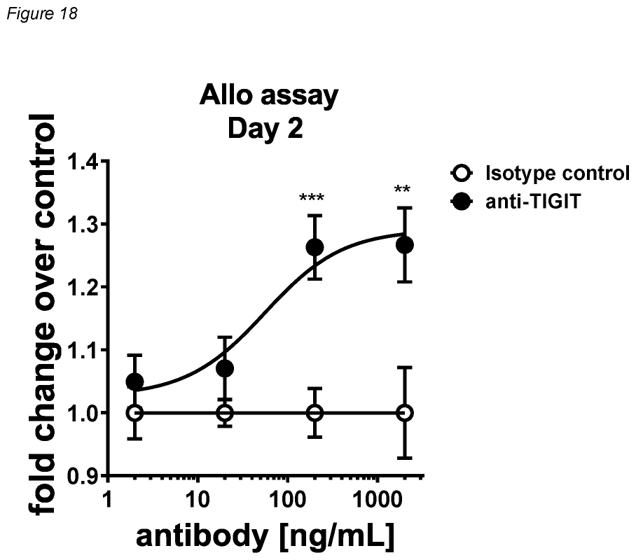

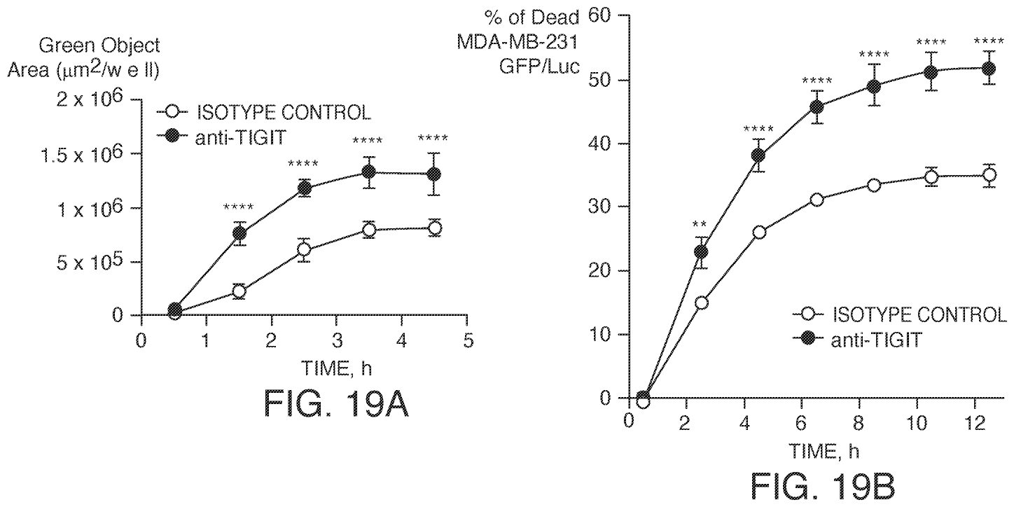

In a one-way MLR assay with cells from two unrelated donors, responder (effector T) cells undergo activation and proliferation in response to the major histocompatibility antigen (MHC Class I and II) differences between the responder cells and stimulator (target) cells. Co-treatment with a functional antagonist checkpoint inhibitor (CPI) antibody further potentiates T cell activation, measured by IFNγ production. Irradiated MDA-MB-231 tumor cells were co-cultured with PBMCs from a human donor for 7 days using IL-2 (R&D Systems, IL-010) to induce allogenic reactive T cell expansion. These cells (effector cells) were then harvested and co-cultured at a 2:1 E:T ratio with freshly irradiated MDA-MB-231 cells (target cells) and co-treated with anti-TIGIT and/or anti-PD-L1 (avelumab) antibodies. T cell activation was evaluated by measuring IFN-γ in the supernatant. Co-cultured cells were treated with serial dilutions of 3963H03-12 or isotype control. Results from 2 assays were plotted together as fold charges over isotype control at 1 ng/mL which was set to 1. 3963H03-12 dose-dependently enhanced Allo-antigen specific T cell activation, with an EC50 of 136.9±114.6 ng/mL (0.9±0.8 nM) (FIG. 18). For combination studies, co-cultured cells were treated with serial dilutions of avelumab combined with 10 μg/mL of isotype control or H03-12. Combination of H03-12 with avelumab further enhanced IFNγ production (FIG. 19B).

3.17 NK Cell Killing Assay

The ability of 3963H03-12, to enhance NK-mediated tumor cell killing by blocking TIGIT/CD155 interaction was demonstrated using a P815 cell line modified to express human CD155. NK cells were co-cultured with P815.hCD155 cells in the presence of 10 μg/mL of 3963H03-12 or IgG1 isotype control antibody. Tumor cell death was monitored by measuring green signal (Caspase-3/7) using IncuCyte system for 4.5 hours. Cell killing was monitored in four fields, p values for two-way ANOVA comparison between IgG1 control treated and anti-TIGIT antibody treated: p<0.00005 (****). 3963H03-12 increased tumor cell killing up to 2-fold compared with isotype control (FIG. 19A). The ability of anti-TIGIT, H03-12, to enhance NK-mediated tumor cell killing was further demonstrated using breast cancer MDA-MB-231 cell line expressing GFP-reporter. NK cells were co-cultured with MDA-MB-231 GFP/Luc cells in the presence of 10 μg/mL of anti-TIGIT H03-12 or IgG1 control antibody. Tumor cell killing was monitored by measuring GFP signal using IncuCyte system. GFP signal at each time point was normalized to 0-time point. Cell killing was monitored in four fields, p values for two-way ANOVA comparison between IgG1 control and anti-TIGIT H03-12 antibody: p<0.00005 (****), p<0.005 (**). Significant increase of NK-mediated tumor cells death by anti-TIGIT H03-12 was detected from 2.5 to 12.5 hours (FIG. 19B).

4. In Vivo Activity

4.1 Blocking Assay of 3963H03-12 and 3963H03-12-muIgG2c on the Binding of Mouse CD155 (muCD155) and Mouse CD112 (muCD112) to CHO-s-huTIGIT Cells

To evaluate the efficacy of 3963H03-12 in vivo, a version of 3963H03-12 with a murine immunoglobulin (3963H03-12-muIgG2c) was developed.

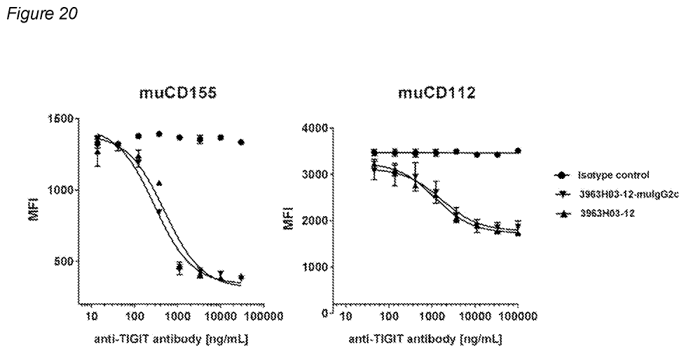

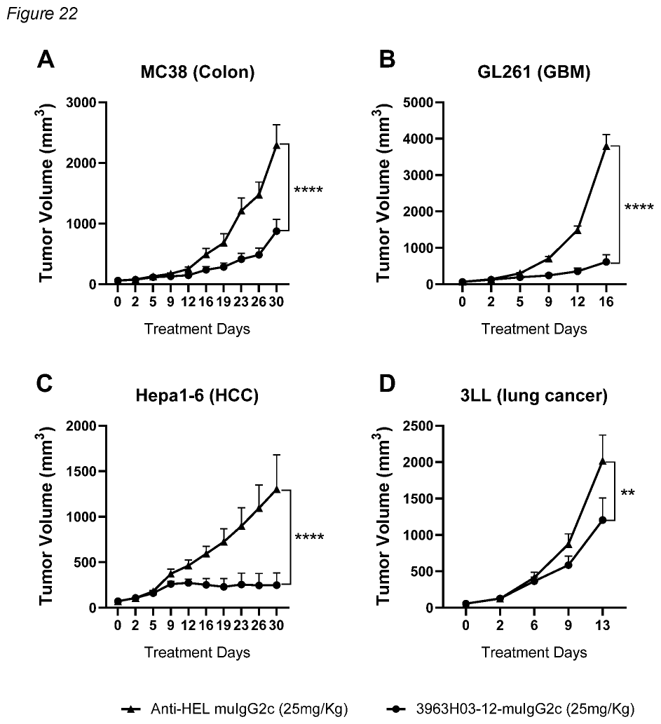

The ability of 3963H03-12-muIgG2c and 3963H03-12 to block the interaction of TIGIT with its ligands muCD155 and muCD112 was evaluated with a flow cytometry-based binding assay using CHO-S engineered cells stably expressing human TIGIT (CHO-S-hTIGIT cell line). Pre-incubation with 3963H03-12-muIgG2c and 3963H03-12, but not the isotype control, led to reduced binding of muCD155-Fc to CHO-hTIGIT cells. 3963H03-12-muIgG2c and 3963H03-12 both dose-dependently blocked the interaction of TIGIT with muCD155 with an IC50 of 290.7 ng/mL (1.994 nM) and 499.3 ng/mL (3.450 nM), respectively (FIG. 22). 3963H03-12-muIgG2c and 3963H03-12 also both dose-dependently blocked the interaction of TIGIT with muCD112 with an IC50 of 1189 ng/mL (8.155 nM) and 1678 ng/mL (11.593 nM), respectively (FIG. 20).

4.2 Pharmacokinetic Evaluation of 3963H03-12-muIgG2c in B-huTIGIT Knock-In Mice Bearing MC38 Tumor

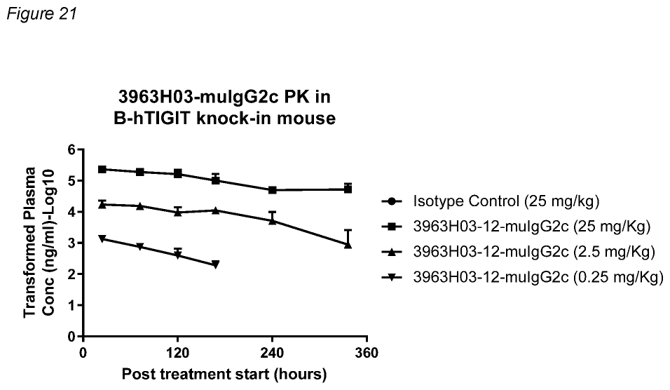

The PK of 3963H03-12-muIgG2c was measured in MC38 tumor-bearing B-huTIGIT knock-in mice developed by Biocytogen. In this model, mouse TIGIT has been replaced with human TIGIT via extreme genome editing (EGE). After a single ip administration, 3963H03-12-muIgG2c peak plasma concentration was measured at the 24 hours in all dose groups. After Cmax, different PK profiles were observed in the three dose groups. Slow monophasic elimination was observed in the high dose group, while fast monophasic elimination was observed in the low dose group. Biphasic elimination was observed in the intermediate dose group, with a slow concentration decrease up to 168 hours followed by a faster decrease up to the last quantifiable time point (336 hours). In line with the PK profile, the calculated terminal half-life was comparable for the low and intermediate dose groups and longer for the high dose group. The AUC 0-∞ increased higher than dose proportionally from 0.25 to 25 mg/kg with the AUC 0-∞ ratio values being 1.0:26.8:334 vs the actual dose ratio of 1:10:100. The increase was roughly dose proportional from 2.5 to 25 mg/kg, where the AUC 0-∞ ratio was 1.0:15.0 vs the actual dose ratio of 1:10 (FIG. 21)

4.3 Anti-Tumor Efficacy of 3963H03-12-muIgG2c in the MC38, GL261, Hepa 1-6, and 3LL Tumor Models in B-huTIGIT Knock-In Mice

The anti-tumor efficacy of 3963H03-12-muIgG2c was evaluated in B-hTIGIT knock-in mice (C57BL/6 background). Female B-huTIGIT knock-in mice, 10 weeks old, were supplied by Biocytogen. The colon cancer cell line MC38, glioblastoma multiforme (GBM) cell line GL261, hepatocellular carcinoma (HCC) cell line Hepa 1-6 and lung cancer cell line 3LL were inoculated subcutaneously at the right upper flank. The inoculated cell amount was 1×10e6, 5×10e6, 2×10e6 and 5×10e6 respectively.

3963H03-12-muIgG2c and anti-HEL-muIgG2c were administered at 25 mg/kg on days 0, 7, 14 via i.p. injection into tumor-bearing B-huTIGIT knock-in mice when tumor size reached 50-100 mm3. Tumor sizes were measured twice per week in three dimensions using a caliper, and the volume was expressed in mm3 using the formula: width×length×height×0.5236. The % TGI is defined as the formula: TGI (%)=[1−(Ti−T0)/(Vi−V0)]×100; Ti is the average tumor volume of a treatment group on a given day, T0 is the average tumor volume of the treatment group on the first day of treatment, Vi is the average tumor volume of the vehicle control group on the same day with Ti, and V0 is the average tumor volume of the vehicle group on the first day of treatment. Two-way ANOVA was performed for significance analysis.

The results are shown in FIG. 22. Compared with the anti-HEL isotype control (binds to hen egg lysozyme), the TGI of 3963H03-12-muIgG2c was 63.5% in MC38 colon cancer (p<0.0001), 85.3% in GL261 GBM (p<0.0001), 85.7% in Hepa 1-6 liver cancer (p<0.0001) and 41.5% in 3LL lung cancer (p=0.0034). Taken together, these data suggest that 3963H03-12-muIgG2c showed anti-tumor efficacy in multiple tumor models, indicating the anti-tumor effect in extensive indications.

4.4 Dose-Dependent Anti-Tumor Efficacy of 3963H03-12-muIgG2c in the MC38 Tumor Model in B-huTIGIT Knock-In Mice

The B-huTIGIT knock-in mice were used to evaluate the anti-tumor efficacy of 3963H03-12-muIgG2c. To optimize the best therapeutic dosage, 3963H03-12-muIgG2c was administered at 25, 5, 1, or 0.2 mg/kg on days 0, 7, 14 via i.p. injection into MC38 tumor-bearing B-huTIGIT knock-in mice. Methods for tumor inoculation and tumor size measurement were as same as described in section 4.3.

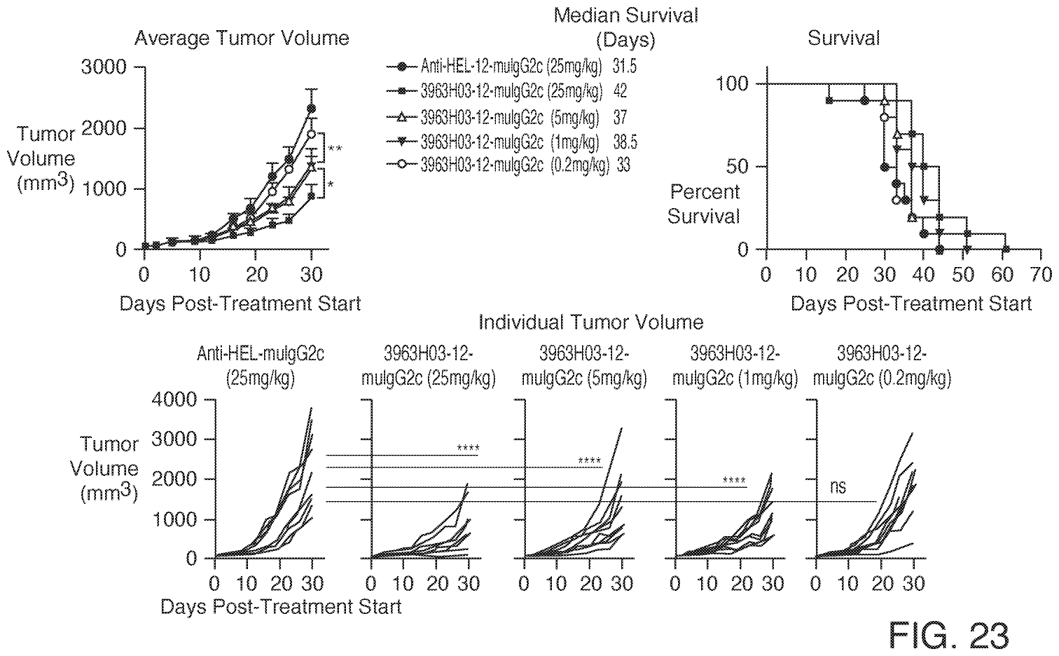

Compared with anti-HEL isotype control, 3963H03-12-muIgG2c at 25 mg/kg, 5 mg/kg, and 1 mg/kg induced significant tumor growth inhibition (TGI=63.5%, 41%, and 42.3%, respectively, and P<0.0001 for each of the three groups, day 30), and prolonged median survival (42, 37, and 38.5 days, respectively) relative to isotype control (31.5 days). Conversely, 0.2 mg/kg of 3963H03-12-muIgG2c did not show significant tumor growth inhibition (TGI=17.6%, P>0.05, day 30) relative to isotype control (FIG. 23).

Although there was not a significant difference in tumor volume between mice treated with 3963H03-12-muIgG2c at 5 mg/kg and those treated with 1 mg/kg (P>0.05, day 30), there was a significant decrease in tumor volume with both 5 mg/kg and 1 mg/kg doses relative to the 0.2 mg/kg dose of 3963H03-12-muIgG2c (P=0.0075 and P=0.0039, respectively, day 30). There was also a significant decrease in tumor volume with 25 mg/kg of 3963H03-12-muIgG2c relative to either 5 mg/kg or 1 mg/kg dose (P=0.0118 and P=0.0211, respectively, day 30). Taken together, these data suggest that 3963H03-12-muIgG2c had dose-dependent anti-tumor efficacy in this tumor model.

4.5 Contribution of Antibody Fc-Mediated Effector Function to the Anti-Tumor Effector of 3963H03-12

The anti-tumor activity of 3963H03-12-muIgG2c and 3963H03-12-muIgG1(D265A) were compared in MC38 and Hepa 1-6 tumor-bearing mice in B-huTIGIT knock-in mice. Female B-huTIGIT knock-in mice, 10 weeks old, were supplied by Biocytogen. Each mouse was inoculated subcutaneously (sc) in the right flank with MC38 tumor cells (1×10e6) in 0.1 mL of PBS or Hepa 1-6 tumor cells (5×10e6) in 0.1 mL of PBS for tumor development. Mice were assigned to treatment groups using stratified randomization based on tumor volume when the average tumor volume reached approximately 50 mm3. There were 10 mice in each group. Mice were treated with anti-HELmuIgG2c (25 mg/kg) or 3963H03-12-muIgG2c (25 mg/kg) or 3963H03-12-muIgG1(D265A) (25 mg/kg) were given at days 0, 7, 14 via ip injection. Tumor size measurement and data analysis protocol were same as described in section 4.3.

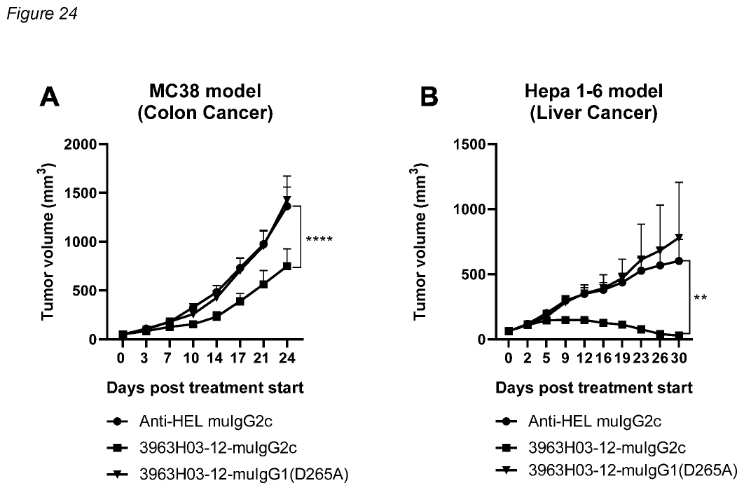

To assess the role of antibody Fc-mediated effector function in the anti-tumor efficacy of 3963H03-12, MC38 and Hepa 1-6 tumor-bearing mice were treated with either effector competent 3963H03-12-muIgG2c or with effector null 3963H03-12-muIgG1(D265A). The immune effector functions of 3963H03-12-muIgG1(D265A) were abolished to reduce FcγR activation and Fc-mediated toxicity. 3963H03-12-muIgG1(D265A) shared many functional characteristics of 3963H03-12-muIgG2c, but it is an ‘effector-silent’ version and cannot induce cytotoxicity effector function. Effector competent 3963H03-12-muIgG2c treatment resulted in significant tumor inhibition in both MC38 and Hepa 1-6 model (TGI=46.82%, P<0.0001, day 24; and TGI=106.45%, P=0.0087, day 30, respectively) compared with isotype control, while the anti-tumor efficacy of effector null 3963H03-12-muIgG1(D265A) (TGI=−4.88% and TGI=−33.07% respectively) was significantly less than effector competent (P<0.0001, day 24 and p=0.0002, day 30, respectively) and not significantly enhanced relative to isotype control (FIG. 24). These results demonstrated that the Fc-mediated immune effector function plays an important role in the anti-tumor efficacy of 3963H03-12-muIgG2c.

4.6. Combination Treatment with 3963H03-12-muIgG2c and Avelumab in the MC38 Tumor Model in B-huTIGIT Knock-In Mice

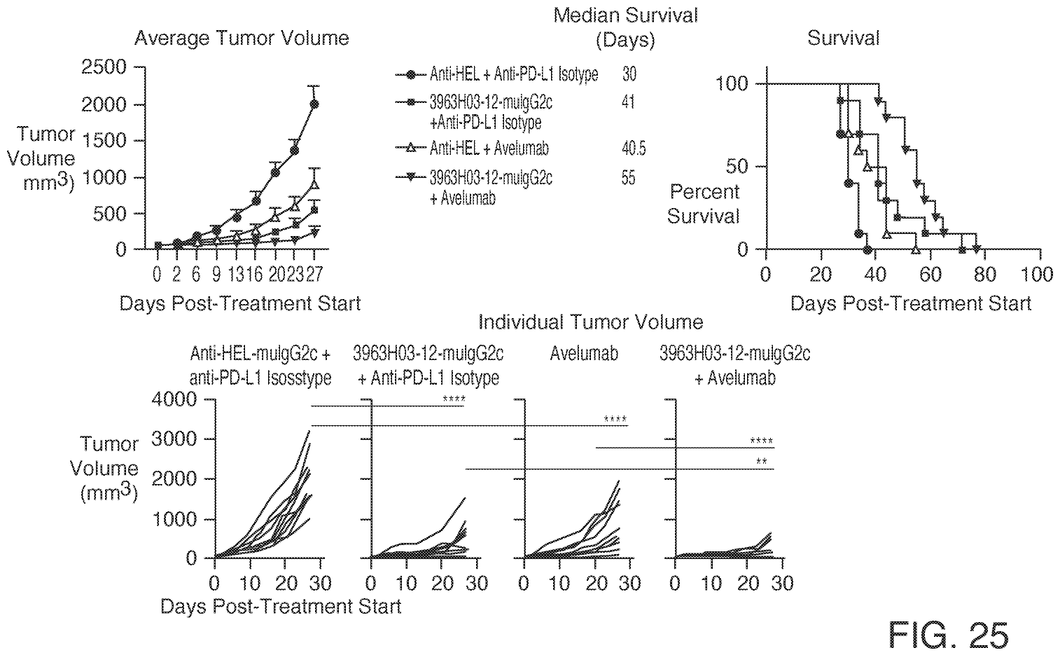

The anti-tumor efficacy of 3963H03-12-muIgG2c in combination with avelumab was also evaluated in MC38 tumor-bearing B-huTIGIT knock-in mice. Compared with anti-HEL+anti-PD-L1 isotype controls, 3963H03-12-muIgG2c and avelumab monotherapies all induced significant tumor growth inhibition (TGI=75.3%, and 56.7% respectively, P<0.0001 for each group relative to isotype control, day 27), and prolonged median survival (41 and 40.5 respectively) relative to isotype control (30 days) (FIG. 25). Tumor growth inhibition was further enhanced with the combination of 3963H03-12-muIgG2c with avelumab (TGI=90.5%) relative to 3963H03-12-muIgG2c (P=0.0028, day 27) and avelumab (P<0.0001, day 27) monotherapies. Combinations of 3963H03-12-muIgG2c with avelumab also prolonged median survival (55 days) (FIG. 25).

4.6 Combination Treatment with 3963H03-12-muIgG2c and M7824 in the MC38 Tumor Model in B-huTIGIT Knock-In Mice

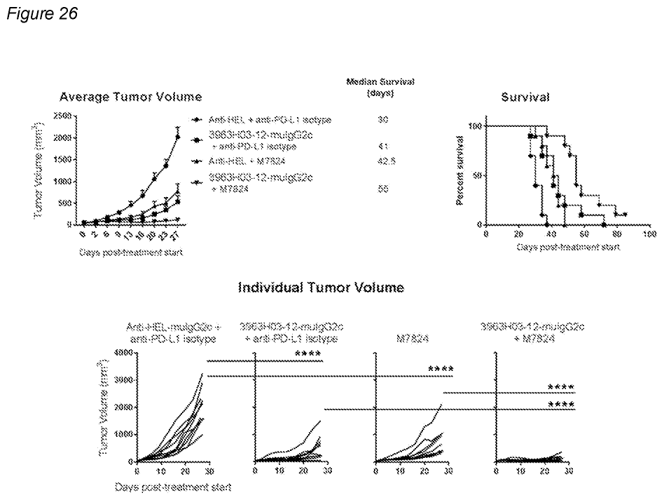

The anti-tumor efficacy of 3963H03-12-muIgG2c in combination with bintrafusp alfa (M7824) was also evaluated in MC38 tumor-bearing B-huTIGIT knock-in mice. Compared with anti-HEL+inactive anti-PD-L1 isotype controls, 3963H03-12-muIgG2c and M7824 monotherapies all induced significant tumor growth inhibition (TGI=75.3% and 63.3%, respectively, P<0.0001 for each group relative to isotype control, day 27), and prolonged median survival (41 and 42.5 days, respectively) relative to isotype control (30 days) (FIG. 26). Tumor growth inhibition was further enhanced with the combination of 3963H03-12-muIgG2c with M7824 (TGI=96.6%) relative to 3963H03-12-muIgG2c (P=0.0011, day 27) and M7824 (P<0.0001, day 27) monotherapies. Combinations of 3963H03-12-IgG2c with M7824 also prolonged median survival (55 days) (FIG. 26).

4.7 Re-Challenge Study

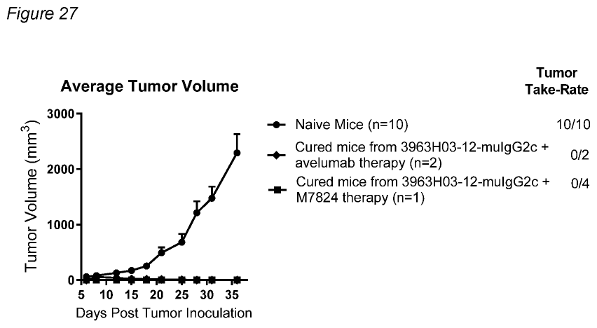

Re-challenge studies were then performed on MC38 tumor-bearing B-huTIGIT knock-in mice that showed complete tumor regression for at least 3 months after 3963H03-12 muIgG2c and avelumab or bintrafusp alfa combination therapy. Mice that were ‘cured’ after 3963H03-12-muIgG2c and avelumab or bintrafusp alfa combination therapy from multiple studies (n=2, n=4 respectively) were re-challenged with MC38 tumor cells in the opposite side of the initial injection. None of these mice developed tumors (0/2 or 0/4 mice, respectively, 0%) for at least 36 days, whereas naïve B-huTIGIT knock-in mice (n=10) injected with MC38 cells all developed tumors (10/10, 100%) (see FIG. 27). These results suggested that 3963H03-12-muIgG2c and avelumab or bintrafusp alfa combination treatment conferred a tumor antigen specific long-term protective immunity in B-huTIGIT knock-in mice.

4.5 Combination Treatment with 3963H03-12-muIgG2c and Avelumab in the MC38 Tumor Model in B-huTIGIT Knock-In Mice

The anti-tumor efficacy of 3963H03-12-muIgG2c in combination with avelumab was also evaluated in MC38 tumor-bearing B-huTIGIT knock-in mice. Compared with anti-HEL+inactive anti-PD-L1 isotype controls, 3963H03-12-muIgG2c and avelumab monotherapies all induced significant tumor growth inhibition (TGI=75.3%, and 56.7% respectively, P<0.0001 for each group relative to isotype control, day 27), and prolonged median survival (41 and 40.5 respectively) relative to isotype control (30 days) (FIG. 26). Tumor growth inhibition was further enhanced with the combination of 3963H03-12-muIgG2c with avelumab (TGI=90.5%) relative to 3963H03-12-muIgG2c (P=0.0028, day 27) and avelumab (P<0.0001, day 27) monotherapies. Combinations of 3963H03-12-muIgG2c with avelumab also prolonged median survival (55 days) (FIG. 25).

4.6 Combination Treatment with 3963H03-12-muIgG2c and M7824 in the MC38 Tumor Model in B-huTIGIT Knock-In Mice

The anti-tumor efficacy of 3963H03-12-muIgG2c in combination with bintrafusp alfa (M7824) was also evaluated in MC38 tumor-bearing B-huTIGIT knock-in mice. Compared with anti-HEL+inactive anti-PD-L1 isotype controls, 3963H03-12-muIgG2c and M7824 monotherapies all induced significant tumor growth inhibition (TGI=75.3% and 63.3%, respectively, P<0.0001 for each group relative to isotype control, day 27), and prolonged median survival (41 and 42.5 days, respectively) relative to isotype control (30 days) (FIG. 26). Tumor growth inhibition was further enhanced with the combination of 3963H03-12-muIgG2c with M7824 (TGI=96.6%) relative to 3963H03-12-muIgG2c (P=0.0011, day 27) and M7824 (P<0.0001, day 27) monotherapies. Combinations of 3963H03-12-IgG2c with M7824 also prolonged median survival (55 days) (FIG. 26).

4.7 Re-Challenge Study

Re-challenge studies were then performed on MC38 tumor-bearing B-huTIGIT knock-in mice that showed complete tumor regression for at least 3 months after 3963H03-12 muIgG2c and avelumab or bintrafusp alfa combination therapy. Mice that were ‘cured’ after 3963H03-12-muIgG2c and avelumab or bintrafusp alfa combination therapy (n=2, 1 respectively) were re-challenged with MC38 tumor cells in the opposite side of the initial injection. None of these mice developed tumors (0/2 or 0/1 mice, respectively, 0%) for at least 36 days, whereas naïve B-huTIGIT knock-in mice (n=10) injected with MC38 cells all developed tumors (10/10, 100%) (see FIG. 27). These results suggested that 3963H03-1-muIgG2c and avelumab or bintrafusp alfa combination treatment conferred a tumor antigen specific protective immunity in B-huTIGIT knock-in mice.

Sequence Listing

| SEQ ID NO. | Sequence | Description |
|---|---|---|
| 1 | MRWCLLLIWAQGLRQAPLASGMMTGTIETTGNISAEKGGSIILQC | Amino acid |
| | HLSSTTAQVTQVNWEQQDQLLAICNADLGWHISPSFKDRVAPGPG | sequence of human |
| | LGLTLQSLTVNDTGEYFCIYHTYPDGTYTGRIFLEVLESSVAEHG | TIGIT |
| | ARFQIPLLGAMAATLVVICTAVIVVVALTRKKKALRIHSVEGDLR | |
| | RKSAGQEEWSPSAPSPPGSCVQAEAAPAGLCGEQRGEDCAELHDY | |
| | FNVLSYRSLGNCSFFTETG | |
| 2 | QVQLVQSGSELKKPGASVKVSCKAS | HC-FR1 |
| 3 | MNVWRQAPGQGLEWMGW | HC-FR2 |
| 4 | TYAQGFTGRFVFSLDTSVSTAYLQISSLKAEDTAVYYC | HC-FR3 |
| 5 | WGQGTLVTVSS | HC-FR4 |
| 6 | QGISSY | HVR-L1 |
| 7 | AAS | HVR-L2 |
| 8 | QQLSSYPT | HVR-L3 |
| 9 | DIQLTQSPSFLSASVGDRVTITCRAS | LC-FR1 |
| 10 | LAWYQQKPGKAPKLLIY | LC-FR2 |
| 11 | TLQSGVPSRFSGSGSGTEFTLTISSLQPEDFATYYC | LC-FR3 |
| 12 | FGGGTKVEIK | LC-FR4 |
| 13 | GYTFTSYP | HVR-H1 |
| 14 | INTNTGNP | HVR-H2 |
| 15 | ARVGGYSVDEYAFDV | HVR-H3 |
| 16 | QVQLVQSGSELKKPGASVKVSCKASGYTFTSYPMNWVRQAPGQGL | HC variable region |
| | EWMGWINTNTGNPTYAQGFTGRFVFSLDTSVSTAYLQISSLKAED | sequence of an anti- |
| | TAVYYCARVGGYSVDEYAFDVWGQGTLVTVSS | TIGIT antibody |
| 17 | DIQLTQSPSFLSASVGDRVTITCRASQGISSYLAWYQQKPGKAPK | LC variable region |
| | LLIYAASTLQSGVPSRFSGSGSGTEFTLTISSLQPEDFATYYCQQ | sequence of an anti- |
| | LSSYPTFGGGTKVEIK | TIGIT antibody |
| 18 | QVQLVQSGSELKKPGASVKVSCKASGYTFTSYPMNWVRQAPGQGL | HC of antibody H03-12 |
| | EWMGWINTNTGNPTYAQGFTGRFVFSLDTSVSTAYLQISSLKAED | produced from |
| | TAVYYCARVGGYSVDEYAFDVWGQGTLVTVSSASTKGPSVFPLAP | CHO-K1SV cells |
| | SSKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVWDV | |
| | SHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRE | |
| | EMTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| 19 | DIQLTQSPSFLSASVGDRVTITCRASQGISSYLAWYQQKPGKAPK | LC of antibody H03-12 |
| | LLIYAASTLQSGVPSRFSGSGSGTEFTLTISSLQPEDFATYYCQQ | produced from |
| | LSSYPTFGGGTKVEIKRTVAAPSVFIFPPSDEQLKSGTASWCLLN | CHO-K1SV cells |
| | NFYPREAKVQWKVDNALQSGNSQESVTEQDSKDSTYSLSSTLTLS | |
| | KADYEKHKVYACEVTHQGLSSPVTKSFNRGEC | |
| 20 | ATGGAAACAGACACCCTGCTGCTGTGGGTGCTGCTGCTGTGGGTG | Isolated nucleic acid |
| | CCCGGCTCCACAGGCCAGGTGCAGCTGGTGCAGTCCGGCTCCGAG | encoding the HC of |
| | CTGAAGAAACCCGGCGCCTCCGTGAAGGTGTCCTGCAAGGCCTCC | an anti-TIGIT |
| | GGCTACACCTTCACCTCCTACCCCATGAACTGGGTGAGGCAGGCT | antibody |
| | CCTGGCCAGGGACTGGAGTGGATGGGCTGGATCAACACCAACACC | |
| | GGCAACCCTACCTACGCCCAGGGCTTCACCGGCAGGTTCGTGTTC | |
| | TCCCTGGACACCAGCGTGTCCACCGCCTACCTGCAGATCTCCTCC | |
| | CTGAAGGCCGAGGACACCGCCGTGTACTACTGCGCCAGGGTGGGA | |
| | GGCTACTCCGTGGACGAGTACGCCTTCGACGTGTGGGGCCAGGGC | |
| | ACCCTGGTGACCGTGTCCTCCGCTAGCACCAAGGGCCCATCGGTC | |
| | TTCCCCCTGGCACCCTCCTCCAAGAGCACCTCTGGGGGCACAGCG | |
| | GCCCTGGGCTGCCTGGTCAAGGACTACTTCCCCGAACCGGTGACG | |
| | GTGTCGTGGAACTCAGGCGCCCTGACCAGCGGCGTGCACACCTTC | |
| | CCGGCTGTCCTACAGTCCTCAGGACTCTACTCCCTCAGCAGCGTG | |
| | GTGACCGTGCCCTCCAGCAGCTTGGGCACCCAGACCTACATCTGC | |
| | AACGTGAATCACAAGCCCAGCAACACCAAGGTGGACAAGAGAGTT | |
| | GAGCCCAAATCTTGTGACAAAACTCACACATGCCCACCGTGCCCA | |
| | GCACCTGAACTCCTGGGGGGACCGTCAGTCTTCCTCTTCCCCCCA | |
| | AAACCCAAGGACACCCTCATGATCTCCCGGACCCCTGAGGTCACA | |
| | TGCGTGGTGGTGGACGTGAGCCACGAAGACCCTGAGGTCAAGTTC | |
| | AACTGGTACGTGGACGGCGTGGAGGTGCATAATGCCAAGACAAAG | |
| | CCGCGGGAGGAGCAGTACAACAGCACGTACCGTGTGGTCAGCGTC | |
| | CTCACCGTCCTGCACCAGGACTGGCTGAATGGCAAGGAGTACAAG | |
| | TGCAAGGTCTCCAACAAAGCCCTCCCAGCCCCCATCGAGAAAACC | |
| | ATCTCCAAAGCCAAAGGGCAGCCCCGAGAACCACAGGTGTACACC | |
| | CTGCCCCCATCACGGGAGGAGATGACCAAGAACCAGGTCAGCCTG | |
| | ACCTGCCTGGTCAAAGGCTTCTATCCCAGCGACATCGCCGTGGAG | |
| | TGGGAGAGCAATGGGCAGCCGGAGAACAACTACAAGACCACGCCT | |
| | CCCGTGCTGGACTCCGACGGCTCCTTCTTCCTCTATAGCAAGCTC | |
| | ACCGTGGACAAGAGCAGGTGGCAGCAGGGGAACGTCTTCTCATGC | |
| | TCCGTGATGCATGAGGCTCTGCACAACCACTACACGCAGAAGAGC | |
| | CTCTCCCTGTCCCCGGGT | |
| 21 | ATGAGGGCCCTGCTGGCTAGACTGCTGCTGTGCGTGCTGGTCGTG | Isolated nucleic acid |
| | TCCGACAGCAAGGGCGACATCCAGCTGACCCAGTCCCCCTCCTTC | encoding the LC of |
| | CTGTCCGCTTCCGTGGGCGACAGGGTGACCATCACTTGTCGTGCC | an anti-TIGIT |
| | TCCCAGGGCATCTCCTCCTACCTGGCCTGGTACCAGCAGAAGCCC | antibody |
| | GGCAAGGCCCCCAAGCTGCTGATCTACGCCGCTTCCACACTGCAG | |
| | TCCGGCGTGCCCTCCAGGTTTTCCGGATCCGGCTCCGGCACCGAG | |
| | TTCACCCTGACCATCTCCTCCCTGCAGCCCGAGGACTTCGCCACC | |
| | TACTACTGCCAGCAGCTGTCCTCCTACCCCACCTTCGGCGGCGGC | |
| | ACAAAGGTGGAGATCAAGCGTACGGTGGCTGCACCATCTGTCTTC | |
| | ATCTTCCCGCCATCTGATGAGCAGTTGAAATCTGGAACTGCCTCT | |
| | GTTGTGTGCCTGCTGAATAACTTCTATCCCAGAGAGGCCAAAGTA | |
| | CAGTGGAAGGTGGATAACGCCCTCCAATCGGGTAACTCCCAGGAG | |
| | AGTGTCACAGAGCAGGACAGCAAGGACAGCACCTACAGCCTCAGC | |
| | AGCACCCTGACGCTGAGCAAAGCAGACTACGAGAAACACAAAGTC | |
| | TACGCCTGCGAAGTCACCCATCAGGGCCTGAGCTCGCCCGTCACA | |
| | AAGAGCTTCAACAGGGGAGAGTGT | |
| 22 | EIQLVQSGSELKKPGASVKVSCKASGYTFTSYPMNWVRQAPGQGL | HC of 3963H03 |
| | EWMGWINTNTGNPTYAQGFTGRFVFSLDTSVSTAYLQISSLKAED | |
| | TAVYYCARVGGYSVDEYAFDVWGQGTMVTVSSASTKGPSVFPLAP | |
| | SSKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVWDV | |
| | SHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRE | |
| | EMTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| 23 | AIRLTQSPSFLSASVGDRVTITCRASQGISSYLAWYQQKPGKAPK | LC of 3963H03 |
| | LLIYAASTLQSGVPSRFSGSGSGTEFTLTISSLQPEDFATYYCQQ | |
| | LNSYPTFGGGTKVEIKRTVAAPSVFIFPPSDEQLKSGTASVCLLN | |
| | NFYPREAKVQWKVDNALQSGNSQESVTEQDSKDSTYSLSSTLTLS | |
| | KADYEKHKVYACEVTHQGLSSPVTKSFNRGEC | |
| 24 | EVQLVQSGSELKKPGASVKVSCKASGYTFTAYPMNVWRQAPGQGL | A06-VH |
| | EWMGWINTNTGNPTYAQGFTGRFVFSLDTSVSTAYLQISSLKAED | |
| | TAVYYCARVGGYSVYDYAFDIWGQGTMVTVSS | |
| 25 | DIQLTQSPSFLSASVGDRVTITCRASQGISSYLAWYQQKPGKAPK | A06-VL |
| | LLIYAASTLQSGVPSRFSGSGSGTEFTLTISSLQPEDFATYYCQQ | |
| | LNSYPTFGGGTKVEIK | |
| 26 | EIQLVQSGSELKKPGASVKVSCKASGYTFTSYPMNWVRQAPGQGL | C11-VH |
| | EWMGWINTNTGNPTNAQGFTGRFVFSLDTSVSTAYLQISSLKAED | |
| | TAVYYCARVGGYGGYDYAFDIWGQGTMVTVSS | |
| 27 | DIQLTQSPSFLSASVGDRVTITCRASQGISSYLAWYQQKPGKAPK | C11-VL |
| | LLIYAASTLQSGVPSRFSGSGSGTEFTLTISSLQPEDFATYYCQQ | |
| | LNSYPTFGGGTKLEIK | |
| 28 | EIQLVQSGSELKKPGASVKVSCKASGYTFTSYPMNWVRQAPGQGL | H03-VH |
| | EWMGWINTNTGNPTYAQGFTGRFVFSLDTSVSTAYLQISSLKAED | |
| | TAVYYCARVGGYSVDEYAFDVWGQGTMVTVSS | |
| 29 | AIRLTQSPSFLSASVGDRVTITCRASQGISSYLAWYQQKPGKAPK | H03-VL |
| | LLIYAASTLQSGVPSRFSGSGSGTEFTLTISSLQPEDFATYYCQQ | |
| | LNSYPTFGGGTKVEIK | |
| 30 | EVQLVQSGSELKKPGASVKVSCKASGYTFTSYPMNWVRQAPGQGL | D08-VH |
| | EWMGWINTNTGNPTYAQGFTGRFVFSLDTSVSTAYLQISSLKAED | |
| | TAVYYCARTGYSGSYYWFDPWGQGTLVTVSS | |
| 31 | DIRLTQSPSFLSASVGDRVTITCRASQGISSYLAWYQQKPGKAPK | D08-VL |
| | FLIYAASTLQSGVPSRFSGSGSGTEFTLTISSLQPEDFATYYCQQ | |
| | LNSYLTFGQGTRLEIK | |
| 32 | QMQLVQSGSELKKPGASVKVSCKASGYTFTSYPMNWVRQAPGQGL | B03-VH |
| | EWMGWINTNTGNPTYAQGFTGRFVFSLDTSVSTAYLQISSLKAED | |
| | TAVYYCARTGGYSVDEYSFDIWGQGTTVTVSS | |
| 33 | DIQLTQSPSFLSASVGDRVTITCRASQGISSYLAWYQQKPGKAPK | B03-VL |
| | LLIYAASTLQSGVPSRFSGSGSGTEFTLTISSLQPEDFATYYCHQ | |
| | TIFRPTFGGGTKVEIK | |
| 34 | QVQLVQSGSELKKPGASVKVSCKASGYTFTSYPMNVWRQAPGQGL | G05-VH |
| | EWMGWINTNTGNPTYAQGFTGRFVFSLDTSVSTAYLQISSLKAED | |
| | TAVYYCARVGGFTVPEYAFDIWGQGTTVTVSS | |
| 35 | DIRLTQSPSFLSASVGDRVTITCRASQGISSYLAWYQQKPGKAPK | G05-VL |
| | LLIYAASTLQSGVPSRFSGSGSGTEFTLTISSLQPEDFATYYCGQ | |
| | VMRYPAFGGGTKVEIK | |
| 36 | GYTFTX₁YP | HVR-H1 consensus |
| | X₁ is S or A | |
| 37 | ARX₂GX₃X₄X₅X₆X₇X₈X₉X₁₀X₁₁X₁₂X₁₃ | HVR-H3 consensus |
| | X₂ is V or T | |
| | X₃ is G or Y | |
| | X₄ is Y, S or F | |
| | X₅ is S, G or T | |
| | X₆ is V, S or G | |
| | X₇ is D, Y or P | |
| | X₈ is E, D or Y | |
| | X₉ is Y or W | |
| | X₁₀ is A, F or S | |
| | X₁₁ is F or D | |
| | X₁₂ is D or P | |
| | X₁₃ is V, I or absent | |
| 38 | X₁₄QX₁₅X₁₆X₁₇X₁₈X₁₉X₂₀ | HVR-L3 consensus |
| | X₁₄ is Q, G or H | |
| | X₁₅ is L, V o r T | |
| | X₁₆ is N, S, I or M | |
| | X₁₇ is S, R, or F | |
| | X₁₈ is Y or R | |
| | X₁₉ is P or L | |
| | X₂₀ is T or A | |
| 39 | QQLNSYPT | HVR-L3 |
| | | embodiment |

1. An isolated heavy chain variable region polypeptide comprising an HVR-H1, HVR-H2 and HVR-H3 sequence, wherein:
(a) the HVR-H1 sequence is GYTFTX₁YP;
(b) the HVR-H2 sequence is INTNTGNP;
(c) the HVR-H3 sequence is ARX₂GX₃X₄X₅X₆X₇X₈X₉X₁₀X₁₁X₁₂X₁₃;
further wherein: X₁ is S or A; X₂ is V or T; X₃ is G or Y; X₄ is Y, S or F; X₅ is S, G or T; X₆ is V, S or G; X₇ is D, Y or P; X₈ is E, D or Y; X₉ is Y or W; X₁₀ is A, F or S; X₁₁ is F or D;
X₁₂ is D or P; X₁₃ is V, I or absent.

2. The polypeptide of claim 1 wherein X₁ is S or A; X₂ is V or T; X₃ is G or Y; X₄ is Y or S; X₅ is S or G; X₆ is V or S; X₇ is D or Y; X₈ is E; X₉ is Y; X₁₀ is A or F; X₁₁ is F; X₁₂ is D; X₁₃ is V or I.

3. The polypeptide of claim 1 wherein X₁ is S; X₂ is V or T; X₃ is G; X₄ is Y; X₅ is S or G; X₆ is V; X₇ is D or Y; X₈ is E; X₉ is Y; X₁₀ is A; X₁₁ is F; X₁₂ is D; X₁₃ is V or I.

4. The polypeptide of claim 1 wherein X₁ is S; X₂ is V; X₃ is G; X₄ is Y; X₅ is S; X₆ is V; X₇ is D; X₈ is E; X₉ is Y; X₁₀ is A; X₁₁ is F; X₁₂ is D; X₁₃ is V.

5. The polypeptide of any one of claims 1-4 further comprising variable region heavy chain framework sequences HC-FR1, HC-FR2, HC-FR3 and HC-FR4, juxtaposed between the HVRs, thus forming the sequence of the formula: (HC-FR1)-(HVR-H1)-(HC-FR2)-(HVR-H2)-(HC-FR3)-(HVR-H3)-(HC-FR4).

6. The polypeptide of claim 5 wherein the heavy chain framework sequences are derived from human consensus framework sequences.

7. The polypeptide of claim 5 wherein the heavy chain framework sequences are derived from human germline framework sequences.

8. The polypeptide of claim 5 wherein one or more of the heavy chain framework sequences is the following:
HC-FR1 is QVQLVQSGSELKKPGASVKVSCKAS;
HC-FR2 is MNWVRQAPGQGLEWMGW;
HC-FR3 is TYAQGFTGRFVFSLDTSVSTAYLQISSLKAEDTAVYYC;
HC-FR4 is WGQGTLVTVSS.

9. The polypeptide of any one of claims 5-8 further comprising at least a C_{H}1 domain.

10. The polypeptide of claim 9 further comprising a C_{H}2 and a C_{H}3 domain.

11. The isolated heavy chain polypeptide of any one of claims 1-10 in combination with a variable region light chain comprising an HVR-L1, HVR-L2 and HVR-L3, wherein:
(a) the HVR-L1 sequence is QGISSY;
(b) the HVR-L2 sequence is AAS;
(c) the HVR-L3 sequence is X₁₄QX₁₅X₁₆X₁₇X₁₈X₁₉X₂₀;
further wherein X₁₄ is Q, G or H; X₁₅ is L, V or T; X₁₆ is N, S, I or M; X₁₇ is S, R or F; X₁₈ is Y or R; X₁₉ is P or L; X₂₀ is T or A.

12. The polypeptide of claim 11 wherein X₁₄ is Q or G; X₁₅ is L or V; X₁₆ is N or S; X₁₇ is S or R; X₁₈ is Y; X₁₉ is P; X₂₀ is T.

13. The polypeptide of claim 11 wherein X₁₄ is Q; X₁₅ is L; X₁₆ is S; X₁₇ is S; X₁₈ is Y; X₁₉ is P; X₂₀ is T.

14. The polypeptide of any of claims 11-13 further comprising variable region light chain framework sequences LC-FR1, LC-FR2, LC-FR3 and LC-FR4, juxtaposed between the HVRs, thus forming the sequence of the formula: (LC-FR1)-(HVR-L1)-(LC-FR2)-(HVR-L2)-(LC-FR3)-(HVR-L3)-(LC-FR4).

15. The polypeptide of claim 14 wherein the light chain framework sequences are derived from human consensus framework sequences.

16. The polypeptide of claim 14 wherein the light chain framework sequences are derived from human germline framework sequences.

17. The polypeptide of claim 14 wherein the light chain framework sequences are kappa light chain sequences.

18. The polypeptide of claim 14 wherein one or more of the light chain framework sequences is the following:
LC-FR1 is DIQLTQSPSFLSASVGDRVTITCRAS;
LC-FR2 is LAWYQQKPGKAPKLLIY;
LC-FR3 is TLQSGVPSRFSGSGSGTEFTLTISSLQPEDFATYYC;
LC-FR4 is FGGGTKVEIK.

19. The polypeptide of any one of claims 14-18 further comprising a C_{L} domain.

20. An isolated anti-TIGIT antibody or antigen binding fragment thereof comprising a heavy chain and a light chain variable region sequence, wherein:
(a) the heavy chain comprises an HVR-H1, HVR-H2 and HVR-H3, wherein further: (i) the HVR-H1 sequence is GYTFTX₁YP; (ii) the HVR-H2 sequence is INTNTGNP; (iii) the HVR-H3 sequence is ARX₂GX₃X₄X₅X₆X₇X₈X₉X₁₀X₁₁X₁₂X₁₃;
(b) the light chain comprises an HVR-L1, HVR-L2 and HVR-L3, wherein further: (iv) the HVR-L1 sequence is QGISSY; (v) the HVR-L2 sequence is AAS; (vi) the HVR-L3 sequence is X₁₄QX₁₅X₁₆X₁₇X₁₈X₁₉X₂₀;
wherein further X₁ is S or A; X₂ is V or T; X₃ is G or Y; X₄ is Y, S or F; X₅ is S, G or T; X₆ is V, S or G; X₇ is D, Y or P; X₈ is E, D or Y; X₉ is Y or W; X₁₀ is A, F or S; X₁₁ is F or D; X₁₂ is D or P; X₁₃ is V, I or absent; X₁₄ is Q, G or H; X₁₅ is L, V or T; X₁₆ is N, S, I or M; X₁₇ is S, R or F; X₁₈ is Y or R; X₁₉ is P or L; X₂₀ is T or A.

21. The antibody or antibody fragment of claim 20 wherein X₁ is S or A; X₂ is V or T; X₃ is G or Y; X₄ is Y or S; X₅ is S or G; X₆ is V or S; X₇ is D or Y; X₈ is E; X₉ is Y; X₁₀ is A or F; X₁₁ is F; X₁₂ is D; X₁₃ is V or I; X₁₄ is Q or G; X₁₅ is L or V; X₁₆ is N or S; X₁₇ is S or R; X₁₈ is Y; X₁₉ is P; X₂₀ is T.

22. The antibody or antibody fragment of claim 20 wherein X₁ is S; X₂ is V or T; X₃ is G; X₄ is Y; X₅ is S or G; X₆ is V; X₇ is D or Y; X₈ is E; X₉ is Y; X₁₀ is A; X₁₁ is F; X₁₂ is D; X₁₃ is V or I; X₁₄ is Q; X₁₅ is L; X₁₆ is S; X₁₇ is S; X₁₈ is Y; X₁₉ is P; X₂₀ is T.

23. The antibody or antibody fragment of claim 20 wherein X₁ is S; X₂ is V; X₃ is G; X₄ is Y; X₅ is S; X₆ is V; X₇ is D; X₈ is E; X₉ is Y; X₁₀ is A; X₁₁ is F; X₁₂ is D; X₁₃ is V; X₁₄ is Q; X₁₅ is L; X₁₆ is S; X₁₇ is S; X₁₈ is Y; X₁₉ is P; X₂₀ is T.

24. The antibody or antibody fragment of claim 20, wherein
(a) the HVR-H1 sequence is GYTFTSYP,
(b) the HVR-H2 sequence is INTNTGNP,
(c) the HVR-H3 sequence is ARVGGYSVDEYAFDV;
and wherein
(d) the HVR-L1 sequence is QGISSY,
(e) the HVR-L2 sequence is AAS,
(f) the HVR-L3 sequence is QQLSSYPT.

25. The antibody or antibody fragment of any of claims 20-24 further comprising:
(a) variable region heavy chain framework sequences HC-FR1, HC-FR2, HC-FR3 and HC-FR4, juxtaposed between the HVRs, thus forming the sequence of the formula: (HC-FR1)-(HVR-H1)-(HC-FR2)-(HVR-H2)-(HC-FR3)-(HVR-H3)-(HC-FR4), and
(b) variable region light chain framework sequences LC-FR1, LC-FR2, LC-FR3 and LC-FR4, juxtaposed between the HVRs, thus forming the sequence of the formula: (LC-FR1)-(HVR-L1)-(LC-FR2)-(HVR-L2)-(LC-FR3)-(HVR-L3)-(LC-FR4).

26. The antibody or antibody fragment of claim 25 wherein the framework sequences are derived from human consensus framework sequences.

27. The antibody or antibody fragment of claim 25 wherein the framework sequences are derived from human germline framework sequences.

28. The antibody or antibody fragment of claim 25 wherein one or more of the framework sequences is the following:
HC-FR1 is QVQLVQSGSELKKPGASVKVSCKAS;
HC-FR2 is MNWVRQAPGQGLEWMGW;
HC-FR3 is TYAQGFTGRFVFSLDTSVSTAYLQISSLKAEDTAVYYC;
HC-FR4 is WGQGTLVTVSS.

29. The antibody or antibody fragment of claim 25 wherein one or more of the framework sequences is the following:
LC-FR1 sequence is DIQLTQSPSFLSASVGDRVTITCRAS;
LC-FR2 sequence is LAWYQQKPGKAPKLLIY;
LC-FR3 sequence is TLQSGVPSRFSGSGSGTEFTLTISSLQPEDFATYYC;
LC-FR4 sequence is FGGGTKVEIK.

30. The antibody or antibody fragment of claim 25 wherein:
(a) the variable heavy chain framework sequences are the following:
(i)
HC-FR1 is QVQLVQSGSELKKPGASVKVSCKAS;
(ii)
HC-FR2 is MNWVRQAPGQGLEWMGW;
(iii)
HC-FR3 is TYAQGFTGRFVFSLDTSVSTAYLQISSLKAEDTAVYYC;
(iv)
HC-FR4 is WGQGTLVTVSS;
and
(b) the variable light chain framework sequences are the following:
(i) LC-FR1 sequence is DIQLTQSPSFLSASVGDRVTITCRAS;
(ii) LC-FR2 sequence is LAWYQQKPGKAPKLLIY;
(iii) LC-FR3 sequence is TLQSGVPSRFSGSGSGTEFTLTISSLQPEDFATYYC;
(iv) LC-FR4 sequence is FGGGTKVEIK.

31. An isolated anti-TIGIT antibody or antigen binding fragment thereof, having the HC-FR and LC-FR sequences of claim 30, selected from the following:
i) an antibody, wherein the HVR-H1, HVR-H2, HVR-H3 sequences are selected from one of the ID's shown in Table 2, and wherein
(a) the HVR-L1 sequence is QGISSY,
(b) the HVR-L2 sequence is AAS,
(c) the HVR-L3 sequence is QQLNSYPT;
ii) an antibody wherein the HVR-L1, HVR-L2, HVR-L3 sequences are selected from one of the ID's shown in Table 3, and wherein
(a) the HVR-H1 sequence is GYTFTSYP,
(b) the HVR-H2 sequence is INTNTGNP,
(c) the HVR-H3 sequence is ARVGGYSVDEYAFDV; or
iii) an antibody chosen from Table 4.

32. The antibody or antibody fragment of any one of claims 25-31 further comprising at least a C_{H}1 domain.

33. The antibody or antibody fragment of claim 32 further comprising a C_{H}2 and a C_{H}3 domain.

34. The antibody or antibody fragment of any one of claims 25-33 further comprising a C_{L} domain.

35. The antibody of claim 34, wherein the constant region is selected from the group consisting of IgG1, IgG2, IgG3 and IgG4.

36. The antibody of claim 35 wherein the constant region is IgG1.

37. The antibody or antibody fragment of any one of the preceding claims which is a fully human antibody.

38. An isolated anti-TIGIT antibody or antigen binding fragment thereof comprising a heavy chain variable region sequence and a light chain variable region sequence, wherein:
(a) the heavy chain sequence has at least 85% sequence identity to the heavy chain sequence:
QVQLVQSGSELKKPGASVKVSCKASGYTFTSYPMNVRQAPGQGLEWMGWI
NTNTGNPTYAQGFTGRFVFSLDTSVSTAYLQISSLKAEDTAVYYCARVGG
YSVDEYAFDVWGQGTLVTVSS,
and
(b) the light chain sequence has at least 85% sequence identity to the light chain sequence:
DIQLTQSPSFLSASVGDRVTITCRASQGISSYLAWYQQKPGKAPKLLIYA
ASTLQSGVPSRFSGSGSGTEFTLTISSLQPEDFATYYCQQLSSYPTFGGG
TKVEIK.

39. The antibody or antigen binding fragment of claim 38, wherein the sequence identity is at least 86%, 87%, 88%, 89%, 90%, 91%, 92%, 93%, 94%, 95%, 96%, 97%, 98%, 99% or is 100%.

40. The antibody or antigen binding fragment of claim 39, wherein the sequence identity is 100%.

41. An isolated anti-TIGIT antibody wherein the heavy chain is:
QVQLVQSGSELKKPGASVKVSCKASGYTFTSYPMNVRQAPGQGLEWMGW
INTNTGNPTYAQGFTGRFVFSLDTSVSTAYLQISSLKAEDTAVYYCARV
GGYSVDEYAFDVWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPG,
and
(b) the light chain is:
DIQLTQSPSFLSASVGDRVTITCRASQGISSYLAWYQQKPGKAPKLLIYA
ASTLQSGVPSRFSGSGSGTEFTLTISSLQPEDFATYYCQQLSSYPTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

42. The antibody of any one of claims 20-41 wherein the antibody is capable of binding to human and cynomolgus monkey TIGIT.

43. The antibody of any one of claims 20-20 wherein the antibody is capable of blocking the interaction between human, or cynomolgus monkey TIGIT and the respective human, or cynomolgus monkey PVR.

44. The antibody of any of claims 20-43 wherein the antibody binds to human TIGIT with a K_{D} of 10×10⁻⁹ M or less.

45. An isolated anti-TIGIT antibody or antigen binding fragment thereof which binds to a functional epitope comprising residues Q53, T55, Y113 and P114 of human TIGIT.

46. The isolated anti-TIGIT antibody or antigen binding fragment of claim 45 wherein the functional epitope further comprises residues Q56, N70, and H111 of human TIGIT.

47. An isolated anti-TIGIT antibody or antigen binding fragment thereof which binds to a conformational epitope comprising residues T51, A52, Q53, T55, Q56, N70, D72, H111, T112, Y113, P114, and G116 of human TIGIT.

48. An isolated anti-TIGIT antibody or antigen binding fragment thereof wherein the antibody cross-competes for binding to TIGIT with an antibody or antigen binding fragment of any of claims 20-42.

49. A pharmaceutical composition comprising the anti-TIGIT antibody or antigen binding fragment of any of claims 20-48 and at least one pharmaceutically acceptable carrier.

50. An isolated nucleic acid encoding a polypeptide of any one of claims 1-41.

51. An isolated nucleic acid encoding the light chain or a heavy chain sequence of an anti-TIGIT antibody or antigen binding fragment of any one of claims 20-41.

52. An isolated nucleic acid encoding the heavy chain according to claim 41, which nucleic acid has the following sequence:
ATGGAAACAGACACCCTGCTGCTGTGGGTGCTGCTGCTGTGGGTGCCCG
GCTCCACAGGCCAGGTGCAGCTGGTGCAGTCCGGCTCCGAGCTGAAGAA
ACCCGGCGCCTCCGTGAAGGTGTCCTGCAAGGCCTCCGGCTACACCTTC
ACCTCCTACCCCATGAACTGGGTGAGGCAGGCTCCTGGCCAGGGACTGG
AGTGGATGGGCTGGATCAACACCAACACCGGCAACCCTACCTACGCCCA
GGGCTTCACCGGCAGGTTCGTGTTCTCCCTGGACACCAGCGTGTCCACC
GCCTACCTGCAGATCTCCTCCCTGAAGGCCGAGGACACCGCCGTGTACT
ACTGCGCCAGGGTGGGAGGCTACTCCGTGGACGAGTACGCCTTCGACGT
GTGGGGCCAGGGCACCCTGGTGACCGTGTCCTCCGCTAGCACCAAGGGC
CCATCGGTCTTCCCCCTGGCACCCTCCTCCAAGAGCACCTCTGGGGGCA
CAGCGGCCCTGGGCTGCCTGGTCAAGGACTACTTCCCCGAACCGGTGAC
GGTGTCGTGGAACTCAGGCGCCCTGACCAGCGGCGTGCACACCTTCCCG
GCTGTCCTACAGTCCTCAGGACTCTACTCCCTCAGCAGCGTGGTGACCG
TGCCCTCCAGCAGCTTGGGCACCCAGACCTACATCTGCAACGTGAATCA
CAAGCCCAGCAACACCAAGGTGGACAAGAGAGTTGAGCCCAAATCTTGT
GACAAAACTCACACATGCCCACCGTGCCCAGCACCTGAACTCCTGGGGG
GACCGTCAGTCTTCCTCTTCCCCCCAAAACCCAAGGACACCCTCATGAT
CTCCCGGACCCCTGAGGTCACATGCGTGGTGGTGGACGTGAGCCACGAA
GACCCTGAGGTCAAGTTCAACTGGTACGTGGACGGCGTGGAGGTGCATA
ATGCCAAGACAAAGCCGCGGGAGGAGCAGTACAACAGCACGTACCGTGT
GGTCAGCGTCCTCACCGTCCTGCACCAGGACTGGCTGAATGGCAAGGAG
TACAAGTGCAAGGTCTCCAACAAAGCCCTCCCAGCCCCCATCGAGAAAA
CCATCTCCAAAGCCAAAGGGCAGCCCCGAGAACCACAGGTGTACACCCT
GCCCCCATCACGGGAGGAGATGACCAAGAACCAGGTCAGCCTGACCTGC
CTGGTCAAAGGCTTCTATCCCAGCGACATCGCCGTGGAGTGGGAGAGCA
ATGGGCAGCCGGAGAACAACTACAAGACCACGCCTCCCGTGCTGGACTC
CGACGGCTCCTTCTTCCTCTATAGCAAGCTCACCGTGGACAAGAGCAGG
TGGCAGCAGGGGAACGTCTTCTCATGCTCCGTGATGCATGAGGCTCTGC
ACAACCACTACACGCAGAAGAGCCTCTCCCTGTCCCCGGGT.

53. An isolated nucleic acid encoding the light chain according to claim 41, which nucleic acid has the following sequence:
ATGAGGGCCCTGCTGGCTAGACTGCTGCTGTGCGTGCTGGTCGTGTCCG
ACAGCAAGGGCGACATCCAGCTGACCCAGTCCCCCTCCTTCCTGTCCGC
TTCCGTGGGCGACAGGGTGACCATCACTTGTCGTGCCTCCCAGGGCATC
TCCTCCTACCTGGCCTGGTACCAGCAGAAGCCCGGCAAGGCCCCCAAGC
TGCTGATCTACGCCGCTTCCACACTGCAGTCCGGCGTGCCCTCCAGGTT
TTCCGGATCCGGCTCCGGCACCGAGTTCACCCTGACCATCTCCTCCCTG
CAGCCCGAGGACTTCGCCACCTACTACTGCCAGCAGCTGTCCTCCTACC
CCACCTTCGGCGGCGGCACAAAGGTGGAGATCAAGCGTACGGTGGCTGC
ACCATCTGTCTTCATCTTCCCGCCATCTGATGAGCAGTTGAAATCTGGA
ACTGCCTCTGTTGTGTGCCTGCTGAATAACTTCTATCCCAGAGAGGCCA
AAGTACAGTGGAAGGTGGATAACGCCCTCCAATCGGGTAACTCCCAGGA
GAGTGTCACAGAGCAGGACAGCAAGGACAGCACCTACAGCCTCAGCAGC
ACCCTGACGCTGAGCAAAGCAGACTACGAGAAACACAAAGTCTACGCCT
GCGAAGTCACCCATCAGGGCCTGAGCTCGCCCGTCACAAAGAGCTTCAA
CAGGGGAGAGTGT.

54. A vector comprising the nucleic acid of any of claims 50-53.

55. A host cell comprising the vector of claim 54.

56. The host cell of claim 55 which is eukaryotic.

57. The host cell of claim 56 which is mammalian.

58. The host cell of claim 57 which is a Chinese Hamster Ovary (CHO) cell, preferably CHO-K1SV.

59. A process for making an anti-TIGIT antibody or antigen binding fragment thereof comprising culturing the host cell of any one of claims 55-58 under conditions suitable for the expression of the vector encoding the anti-TIGIT antibody or antigen binding fragment, and recovering the antibody or fragment.

60. A method of treating cancer comprising administering to a subject in need thereof an effective amount of an anti-TIGIT antibody of any one of claims 20-48, or the pharmaceutical composition of claim 49, which induces antibody dependent cell-mediated cytotoxicity (ADCC).

61. A method of treating cancer comprising administering to a subject in need thereof an effective amount of an anti-TIGIT antibody of any one of claims 20-48, or the pharmaceutical composition of claim 49.

62. The method of claim 60 or 61 wherein the cancer is selected from the group consisting of: breast, lung, colon, ovarian, melanoma, bladder, kidney, liver, salivary, stomach, gliomas, thyroid, thymic, epithelial, head and neck cancers, gastric and pancreatic cancer.

63. A method of treating a T-cell dysfunctional disorder comprising administering a therapeutically effective amount of an anti-TIGIT antibody of any one of claims 20-48 or the pharmaceutical composition of claim 47, to a subject in need thereof.

64. The method of claim 63, wherein the T-cell dysfunctional disorder is tumor immunity.

65. The method of claim 64, wherein the tumor immunity results from a cancer selected from the group consisting of: breast, lung, colon, ovarian, melanoma, bladder, kidney, liver, salivary, stomach, gliomas, thyroid, thymic, epithelial, head and neck cancers, gastric and pancreatic cancer.

66. The method of any one of claims 60-65, wherein the method further comprises the application of a treatment regimen selected from the group consisting of: radiation therapy, surgery, chemotherapy, gene therapy, DNA therapy, viral therapy, RNA therapy, immunotherapy, bone marrow transplantation, nanotherapy, monoclonal antibody therapy, adjuvant therapy, neoadjuvant therapy, hormonal therapy, angiogenesis inhibition, palliative care.

67. The method of any one of claims 60-66, further comprising the administration of at least one anti-cancer agent.

68. A kit of parts comprising the pharmaceutical composition of claim 49 and a package insert comprising instructions for using the pharmaceutical composition for the treatment according to any one of claims 60-66.

69. A kit of parts comprising the pharmaceutical composition of claim 49, at least one further anti-cancer agent, and a package insert comprising instructions for using the at least one anti-cancer agent in combination with the pharmaceutical composition for the treatment according to any one of claims 60-67.


SEQUENCE LISTING (39 sequences)

SEQ ID NO: 1 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 2 - protein, 25 aa, Artificial Sequence
  source: /note=“Description of Artificial Sequence Synthetic peptide”
QVQLVQSGSELKKPGASVKVSCKAS

SEQ ID NO: 3 - protein, 17 aa, Artificial Sequence
  source: /note=“Description of Artificial Sequence Synthetic peptide”
MNWVRQAPGQGLEWMGW

SEQ ID NO: 4 - protein, 38 aa, Artificial Sequence
  source: /note=“Description of Artificial Sequence Synthetic polypeptide”
TYAQGFTGRFVFSLDTSVSTAYLQISSLKAEDTAVYYC

SEQ ID NO: 5 - protein, 11 aa, Artificial Sequence
  source: /note=“Description of Artificial Sequence Synthetic peptide”
WGQGTLVTVSS

SEQ ID NO: 6 - protein, 6 aa, Artificial Sequence
  source: /note=“Description of Artificial Sequence Synthetic peptide”
QGISSY

SEQ ID NO: 7 - protein, 3 aa, Artificial Sequence
  source: /note=“Description of Artificial Sequence Synthetic peptide”
AAS

SEQ ID NO: 8 - protein, 8 aa, Artificial Sequence
  source: /note=“Description of Artificial Sequence Synthetic peptide”
QQLSSYPT

SEQ ID NO: 9 - protein, 26 aa, Artificial Sequence
  source: /note=“Description of Artificial Sequence Synthetic peptide”
DIQLTQSPSFLSASVGDRVTITCRAS

SEQ ID NO: 10 - protein, 17 aa, Artificial Sequence
  source: /note=“Description of Artificial Sequence Synthetic peptide”
LAWYQQKPGKAPKLLIY

SEQ ID NO: 11 - protein, 36 aa, Artificial Sequence
  source: /note=“Description of Artificial Sequence Synthetic polypeptide”
TLQSGVPSRFSGSGSGTEFTLTISSLQPEDFATYYC

SEQ ID NO: 12 - protein, 10 aa, Artificial Sequence
  source: /note=“Description of Artificial Sequence Synthetic peptide”
FGGGTKVEIK

SEQ ID NO: 13 - protein, 8 aa, Artificial Sequence
  source: /note=“Description of Artificial Sequence Synthetic peptide”
GYTFTSYP

SEQ ID NO: 14 - protein, 8 aa, Artificial Sequence
  source: /note=“Description of Artificial Sequence Synthetic peptide”
INTNTGNP

SEQ ID NO: 15 - protein, 15 aa, Artificial Sequence
  source: /note=“Description of Artificial Sequence Synthetic peptide”
ARVGGYSVDEYAFDV

SEQ ID NO: 16 - protein, 122 aa, Artificial Sequence
  source: /note=“Description of Artificial Sequence Synthetic polypeptide”
QVQLVQSGSELKKPGASVKVSCKASGYTFTSYPMNWVRQAPGQGLEWMGWINTNTGNPTYAQGFTGRFVFSLDTSVSTAYLQISSLKAEDTAVYYCARVGGYSVDEYAFDVWGQGTLVTVSS

SEQ ID NO: 17 - protein, 106 aa, Artificial Sequence
  source: /note=“Description of Artificial Sequence Synthetic polypeptide”
DIQLTQSPSFLSASVGDRVTITCRASQGISSYLAWYQQKPGKAPKLLIYAASTLQSGVPSRFSGSGSGTEFTLTISSLQPEDFATYYCQQLSSYPTFGGGTKVEIK

SEQ ID NO: 18 - protein, 451 aa, Artificial Sequence
  source: /note=“Description of Artificial Sequence Synthetic polypeptide”
QVQLVQSGSELKKPGASVKVSCKASGYTFTSYPMNWVRQAPGQGLEWMGWINTNTGNPTYAQGFTGRFVFSLDTSVSTAYLQISSLKAEDTAVYYCARVGGYSVDEYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 213 aa, Artificial Sequence
  source: /note=“Description of Artificial Sequence Synthetic polypeptide”
DIQLTQSPSFLSASVGDRVTITCRASQGISSYLAWYQQKPGKAPKLLIYAASTLQSGVPSRFSGSGSGTEFTLTISSLQPEDFATYYCQQLSSYPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 1413 bp, Artificial Sequence
  source: /note=“Description of Artificial Sequence Synthetic polynucleotide”
atggaaacagacaccctgctgctgtgggtgctgctgctgtgggtgcccggctccacaggccaggtgcagctggtgcagtccggctccgagctgaagaaacccggcgcctccgtgaaggtgtcctgcaaggcctccggctacaccttcacctcctaccccatgaactgggtgaggcaggctcctggccagggactggagtggatgggctggatcaacaccaacaccggcaaccctacctacgcccagggcttcaccggcaggttcgtgttctccctggacaccagcgtgtccaccgcctacctgcagatctcctccctgaaggccgaggacaccgccgtgtactactgcgccagggtgggaggctactccgtggacgagtacgccttcgacgtgtggggccagggcaccctggtgaccgtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 21 - DNA, 699 bp, Artificial Sequence
  source: /note=“Description of Artificial Sequence Synthetic polynucleotide”
atgagggccctgctggctagactgctgctgtgcgtgctggtcgtgtccgacagcaagggcgacatccagctgacccagtccccctccttcctgtccgcttccgtgggcgacagggtgaccatcacttgtcgtgcctcccagggcatctcctcctacctggcctggtaccagcagaagcccggcaaggcccccaagctgctgatctacgccgcttccacactgcagtccggcgtgccctccaggttttccggatccggctccggcaccgagttcaccctgaccatctcctccctgcagcccgaggacttcgccacctactactgccagcagctgtcctcctaccccaccttcggcggcggcacaaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 22 - protein, 451 aa, Artificial Sequence
  source: /note=“Description of Artificial Sequence Synthetic polypeptide”
EIQLVQSGSELKKPGASVKVSCKASGYTFTSYPMNWVRQAPGQGLEWMGWINTNTGNPTYAQGFTGRFVFSLDTSVSTAYLQISSLKAEDTAVYYCARVGGYSVDEYAFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 213 aa, Artificial Sequence
  source: /note=“Description of Artificial Sequence Synthetic polypeptide”
AIRLTQSPSFLSASVGDRVTITCRASQGISSYLAWYQQKPGKAPKLLIYAASTLQSGVPSRFSGSGSGTEFTLTISSLQPEDFATYYCQQLNSYPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 122 aa, Artificial Sequence
  source: /note=“Description of Artificial Sequence Synthetic polypeptide”
EVQLVQSGSELKKPGASVKVSCKASGYTFTAYPMNWVRQAPGQGLEWMGWINTNTGNPTYAQGFTGRFVFSLDTSVSTAYLQISSLKAEDTAVYYCARVGGYSVYDYAFDIWGQGTMVTVSS

SEQ ID NO: 25 - protein, 106 aa, Artificial Sequence
  source: /note=“Description of Artificial Sequence Synthetic polypeptide”
DIQLTQSPSFLSASVGDRVTITCRASQGISSYLAWYQQKPGKAPKLLIYAASTLQSGVPSRFSGSGSGTEFTLTISSLQPEDFATYYCQQLNSYPTFGGGTKVEIK

SEQ ID NO: 26 - protein, 122 aa, Artificial Sequence
  source: /note=“Description of Artificial Sequence Synthetic polypeptide”
EIQLVQSGSELKKPGASVKVSCKASGYTFTSYPMNWVRQAPGQGLEWMGWINTNTGNPTNAQGFTGRFVFSLDTSVSTAYLQISSLKAEDTAVYYCARVGGYGGYDYAFDIWGQGTMVTVSS

SEQ ID NO: 27 - protein, 106 aa, Artificial Sequence
  source: /note=“Description of Artificial Sequence Synthetic polypeptide”
DIQLTQSPSFLSASVGDRVTITCRASQGISSYLAWYQQKPGKAPKLLIYAASTLQSGVPSRFSGSGSGTEFTLTISSLQPEDFATYYCQQLNSYPTFGGGTKLEIK

SEQ ID NO: 28 - protein, 122 aa, Artificial Sequence
  source: /note=“Description of Artificial Sequence Synthetic polypeptide”
EIQLVQSGSELKKPGASVKVSCKASGYTFTSYPMNWVRQAPGQGLEWMGWINTNTGNPTYAQGFTGRFVFSLDTSVSTAYLQISSLKAEDTAVYYCARVGGYSVDEYAFDVWGQGTMVTVSS

SEQ ID NO: 29 - protein, 106 aa, Artificial Sequence
  source: /note=“Description of Artificial Sequence Synthetic polypeptide”
AIRLTQSPSFLSASVGDRVTITCRASQGISSYLAWYQQKPGKAPKLLIYAASTLQSGVPSRFSGSGSGTEFTLTISSLQPEDFATYYCQQLNSYPTFGGGTKVEIK

SEQ ID NO: 30 - protein, 121 aa, Artificial Sequence
  source: /note=“Description of Artificial Sequence Synthetic polypeptide”
EVQLVQSGSELKKPGASVKVSCKASGYTFTSYPMNWVRQAPGQGLEWMGWINTNTGNPTYAQGFTGRFVFSLDTSVSTAYLQISSLKAEDTAVYYCARTGYSGSYYWFDPWGQGTLVTVSS

SEQ ID NO: 31 - protein, 106 aa, Artificial Sequence
  source: /note=“Description of Artificial Sequence Synthetic polypeptide”
DIRLTQSPSFLSASVGDRVTITCRASQGISSYLAWYQQKPGKAPKFLIYAASTLQSGVPSRFSGSGSGTEFTLTISSLQPEDFATYYCQQLNSYLTFGQGTRLEIK

SEQ ID NO: 32 - protein, 122 aa, Artificial Sequence
  source: /note=“Description of Artificial Sequence Synthetic polypeptide”
QMQLVQSGSELKKPGASVKVSCKASGYTFTSYPMNWVRQAPGQGLEWMGWINTNTGNPTYAQGFTGRFVFSLDTSVSTAYLQISSLKAEDTAVYYCARTGGYSVDEYSFDIWGQGTTVTVSS

SEQ ID NO: 33 - protein, 106 aa, Artificial Sequence
  source: /note=“Description of Artificial Sequence Synthetic polypeptide”
DIQLTQSPSFLSASVGDRVTITCRASQGISSYLAWYQQKPGKAPKLLIYAASTLQSGVPSRFSGSGSGTEFTLTISSLQPEDFATYYCHQTIFRPTFGGGTKVEIK

SEQ ID NO: 34 - protein, 122 aa, Artificial Sequence
  source: /note=“Description of Artificial Sequence Synthetic polypeptide”
QVQLVQSGSELKKPGASVKVSCKASGYTFTSYPMNWVRQAPGQGLEWMGWINTNTGNPTYAQGFTGRFVFSLDTSVSTAYLQISSLKAEDTAVYYCARVGGFTVPEYAFDIWGQGTTVTVSS

SEQ ID NO: 35 - protein, 106 aa, Artificial Sequence
  source: /note=“Description of Artificial Sequence Synthetic polypeptide”
DIRLTQSPSFLSASVGDRVTITCRASQGISSYLAWYQQKPGKAPKLLIYAASTLQSGVPSRFSGSGSGTEFTLTISSLQPEDFATYYCGQVMRYPAFGGGTKVEIK

SEQ ID NO: 36 - protein, 8 aa, Artificial Sequence
  source: /note=“Description of Artificial Sequence Synthetic peptide”
  VARIANT (6)..(6): /replace=“Ala”
  SITE (1)..(8): /note=“Variant residues given in the sequence have no preference with respect to those in the annotations for variant positions”
GYTFTSYP

SEQ ID NO: 37 - protein, 15 aa, Artificial Sequence
  source: /note=“Description of Artificial Sequence Synthetic peptide”
  VARIANT (3)..(3): /replace=“Thr”
  VARIANT (5)..(5): /replace=“Tyr”
  VARIANT (6)..(6): /replace=“Ser” or “Phe”
  VARIANT (7)..(7): /replace=“Gly” or “Thr”
  VARIANT (8)..(8): /replace=“Ser” or “Gly”
  VARIANT (9)..(9): /replace=“Tyr” or “Pro”
  VARIANT (10)..(10): /replace=“Asp” or “Tyr”
  VARIANT (11)..(11): /replace=“Trp”
  VARIANT (12)..(12): /replace=“Phe” or “Ser”
  VARIANT (13)..(13): /replace=“Asp”
  VARIANT (14)..(14): /replace=“Pro”
  VARIANT (15)..(15): /replace=“Ile” or “ ”
  SITE (1)..(15): /note=“Variant residues given in the sequence have no preference with respect to those in the annotations for variant positions”
ARVGGYSVDEYAFDV

SEQ ID NO: 38 - protein, 8 aa, Artificial Sequence
  source: /note=“Description of Artificial Sequence Synthetic peptide”
  VARIANT (1)..(1): /replace=“Gly” or “His”
  VARIANT (3)..(3): /replace=“Val” or “Thr”
  VARIANT (4)..(4): /replace=“Ser” or “Ile” or “Met”
  VARIANT (5)..(5): /replace=“Arg” or “Phe”
  VARIANT (6)..(6): /replace=“Arg”
  VARIANT (7)..(7): /replace=“Leu”
  VARIANT (8)..(8): /replace=“Ala”
  SITE (1)..(8): /note=“Variant residues given in the sequence have no preference with respect to those in the annotations for variant positions”
QQLNSYPT

SEQ ID NO: 39 - protein, 8 aa, Artificial Sequence
  source: /note=“Description of Artificial Sequence Synthetic peptide”
QQLNSYPT